(19) United States
(12) Patent Application Publication
(10) Pub. No.: US 2024/0279550 A1
(43) Pub. Date: Aug. 22, 2024

(54) REACTIVE MESOGENS

(71) Applicant: Merck Patent GmbH, Darmstadt (DE)

(72) Inventors: Kevin Adlem, Feltham (DE); Owain Llyr Parri, Feltham (DE); Alex Davis, Feltham (DE); Stephen Mulcahy, Feltham (DE); Philipp Wucher, Darmstadt (DE); Beate Schneider, Darmstadt (DE)

(73) Assignee: Merck Patent GmbH, Darmstadt (DE)

(21) Appl. No.: 18/289,626

(22) PCT Filed: May 4, 2022
(86) PCT No.: PCT/EP2022/061922

§ 371 Date: Nov. 6, 2023

(30) Foreign Application Priority Data
May 7, 2021 (EP) 21172901.7

(51) Int. Cl. C09K 19/18; C07C 69/533; C07C 255/54; C07C 323/09; C07D 209/12; C07D 333/76; C07D 495/04; C09K 19/04; C09K 19/20

(52) U.S. Cl. CPC C09K 19/18; C07C 69/533; C07C 255/54; C07C 323/09; C07D 209/12; C07D 333/76; C07D 495/04; C07C 2602/10; C07C 2603/24; C09K 2019/0448; C09K 2019/2092

(57) ABSTRACT

Reactive mesogens (RMs) derived from tolane, mixtures and formulations comprising them, polymers obtained from such RMs and RM mixtures, and the use of the RMs, RM mixtures and polymers in optical or electrooptical components or devices.

### FIELD OF THE INVENTION

The invention relates to reactive mesogens (RMs), to mixtures and formulations comprising them, to polymers obtained from such RMs and RM mixtures, and the use of the RMs, RM mixtures and polymers in optical or electrooptical components or devices.

### Background and Prior Art

Reactive mesogens (RMs), mixtures or formulations comprising them, and polymers obtained thereof, can be used to make optical components, like compensation, retardation or polarisation films, or lenses. These optical components can be used in optical or electrooptical devices like LC displays. Usually the RMs or RM mixtures are polymerised through the process of in-situ polymerisation.

The manufacture of RM film products with high birefringence is of high importance for manufacturing optical components of modern display devices like LCDs or augmented or virtual reality (AR/VR) applications. For Example, brightness enhancement films such as 3M DBEF™, are often included in displays in order to increase the brightness or reduce the number of light sources in the backlight unit. Broadband cholesteric films can also be used for this purpose, and the optical properties are dependent upon the broadening which can be achieved during processing. Films which are better able to broaden can be processed faster on a production line, and additionally can have improved optical properties.

In this regard, it is possible to polymerise cholesteric reactive mesogen films such that a gradient in the helical pitch is obtained, thereby broadening the reflection band of the film. Thin films with good optical properties are dependent on the inclusion of at least one suitable high birefringence RM.

Broadening of cholesteric films is dictated by the structure of the high birefringence material in the reactive mesogen mixture. Compounds must be highly birefringent and allow band broadening to occur whilst also having good solubility and a broad nematic range, preferably without melting points becoming too high. High birefringence reactive mesogens made to date with these characteristics only allow cholesteric films to be broadened by a certain amount before films become hazy.

Increasing the birefringence of the RM whilst keeping them polymerisable and with good physical properties is possible, but requires the incorporation of specific chemical groups, like for example tolane groups, into the compounds.

Mesogenic tolane derivatives are known for example from U.S. Pat. No. 6,514,578 B1, GB 2 388 599 B1, U.S. Pat. No. 7,597,942 B1, US 2003-072893 A1, US 2006-0119783 A1 or JP 2015-205843.

Generally tolane groups are relatively reactive and are mostly unsuited to light exposure, making them difficult to utilise in many optical applications due to yellowing or other degradation effects. Furthermore, mesogenic tolane derivatives often show a limited solubility in RM mixtures or organic solvents and are therefore limited in their use.

It is therefore an aim of the present invention to provide improved RMs, RM mixtures and RM formulations, which do not have the drawbacks of materials known from prior art. In particular it is an aim to provide RMs and RM mixtures and RM formulations that are suitable for preparing polymers by in situ polymerisation, and exhibit at the same time a high birefringence, exhibit a good solubility, show an improved broadening potential, have favorable transition temperatures, and show high resistance against yellowing after being exposed to UV light. Other aims of the invention are immediately evident to the expert from the following description.

Surprisingly, the inventors of the present invention have found that the polymerizable tolane derivatives according to claim 1 fulfil one or more aims as given above.

### SUMMARY OF THE INVENTION

The instant invention therefore relates to compounds of formula I,

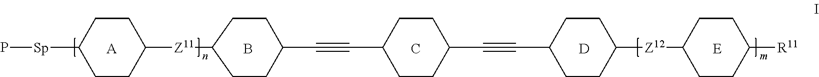

wherein
P is a polymerisable group,
Sp is a spacer group or a single bond,
R¹¹ is F, Cl, CN, NCS, alkyl, alkoxy, thioalkyl, alkylcarbonyl, alkoxycarbonyl, alkylcarbonyloxy or alkoxycarbonyloxy preferably with 1 to 15 C atoms which is more preferably optionally fluorinated or P-Sp,
A, B,
D, and E denote, in case of multiple occurrence independently of one another an alicyclic, heterocyclic, aromatic or heteroaromatic group with 4 to 20 ring atoms, which is monocyclic or polycyclic and which is optionally substituted by one or more groups L or P—Sp—, and one of rings C and D may also denote a single bond,
L F, Cl, —CN, P—Sp—, or straight chain, branched or cyclic alkyl having 1 to 25 C atoms, wherein one or more non-adjacent CH₂— groups are optionally replaced by —O—, —S—, —CO—, —CO—O—, —O—CO—, —O—CO—O—, CR⁰═CR⁰⁰—, —C≡C—,

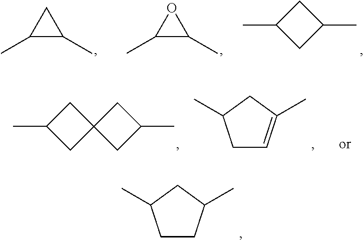

in such a manner that O- and/or S-atoms are not directly connected with each other, and wherein one or more H atoms are each optionally replaced by P—Sp—, F or Cl, or two substituents L that are connected to directly adjacent C atoms may also form a cycloalkyl or cycloalkenyl group with 5, 6, 7 or 8 C atoms,

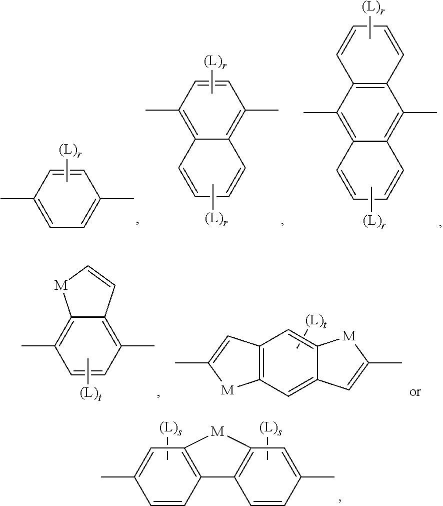

C denotes
M denotes CH₂, C(CH₃)₂, CHF, CF₂, NH, S or O,
Z¹¹, Z¹² denotes, in case of multiple occurrence independently of one another, —O—, —S—, —CO—, —COO—, —OCO—, —S—CO—, —CO—S—, —O—COO—, —CO—NR⁰—, —NR⁰—CO—, —NR⁰—CO—NR⁰⁰, —NR⁰—CO—O—, —O—CO—NR⁰—, —OCH²—, —CH₂O—, —SCH₂—, —CH₂S—, —CF₂O—, —OCF₂—, —CF₂S—, —SCF₂—, —CH₂CH₂—, —(CH₂)ₙ₁, —CF₂CH₂—, —CH₂CF₂—, —CF₂CF₂—, —CH═N—, —N═CH—, —N═N—, —CH═CRO—, —CY²═CY²—, —C═C—, —CH═CH—COO—, —OCO—CH═CH— or a single bond, preferably —COO—, —OCO—, —C≡C—, or a single bond,
n1 is 1, 2, 3 or 4,
r denotes 0, 1, 2, 3 or 4, preferably 0, 1 or 2,
s denotes 0, 1, 2 or 3, preferably 0, 1 or 2, very preferably 0 or 1,
t denotes 0, 1 or 2, preferably 0 or 1, very preferably 0,
R⁰, R⁰⁰ denote H or alkyl having 1 to 12 C atoms,
Y¹, Y² independently of each other denote H, F, Cl, NCS, or CN,
n is 0, 1, 2, 3 or 4, preferably 0, 1 or 2, more preferably 0 or 1, most preferably 0,
m is 0, 1, 2, 3 or 4, preferably 0, 1 or 2, more preferably 0 or 1, most preferably 0.

The invention further relates to a mixture, which is hereinafter referred to as “RM mixture”, comprising two or more RMs, at least one of which is a compound of formula I.

The invention further relates to a formulation, which is hereinafter referred to as “RM formulation”, comprising one or more compounds of formula I or an RM mixture as described above and below, and further comprising one or more solvents and/or additives.

The invention further relates to a polymer obtainable or obtained by polymerising a compound of formula I or an RM mixture as described above and below, preferably wherein the RMs are aligned, and preferably at a temperature where the RMs or RM mixture exhibit a liquid crystal phase.

The invention further relates to the use of the compounds of formula I, the RM mixture or the polymer as described above and below in optical, electrooptical or electronic components or devices.

The invention further relates to an optical, electrooptical or electronic device or a component comprising an RM, RM mixture or a polymer as described above and below.

Said components include, without limitation, optical retardation films, polarizers, compensators, beam splitters, reflective films, alignment layers, colour filters, antistatic protection sheets, electromagnetic interference protection sheets, polarization controlled lenses for example for autostereoscopic 3D displays, IR reflection films for example for window applications, spatial light modulators, and lenses for light guides, focusing and optical effects, eg. 3D, holography, telecomms.

Said devices include, without limitation, electro optical displays, especially LC displays, autostereoscopic 3D displays, organic light emitting diodes (OLEDs), optical data storage devices, googles for AR/VR applications and windows.

### Definitions of Terms

As used herein, the term “RM mixture” means a mixture comprising two, three, four, five six, seven, eight, nine or more RMs.

As used herein, the term “RM formulation” means at least one RM or RM mixture, and one or more other materials added to the at least one RM or RM mixture to provide, or to modify, specific properties of the RM formulation and/or of the at least one RM therein. It will be understood that an RM formulation is also a vehicle for carrying the RM to a substrate to enable the forming of layers or structures thereon. Exemplary materials include, but are not limited to, solvents, polymerisation initiators, surfactants and adhesion promoters, etc. as described in more detail below.

As used herein, the terms “reactive mesogen” and “RM” will be understood to mean a compound containing a mesogenic or liquid crystalline skeleton, and one or more functional groups attached thereto which are suitable for polymerization and are also referred to as “polymerizable group” or “P”.

Unless stated otherwise, the term “polymerizable compound” as used herein will be understood to mean a polymerizable monomeric compound.

The terms “liquid crystal”, “mesogen” and “mesogenic compound” as used herein mean a compound that under suitable conditions of temperature, pressure and concentration can exist as a mesophase or in particular as a LC phase.

The term “mesogenic group” as used herein is known to the person skilled in the art and described in the literature, and means a group which, due to the anisotropy of its attracting and repelling interactions, essentially contributes to causing a liquid-crystal (LC) phase in low-molecular-weight or polymeric substances. Compounds containing mesogenic groups (mesogenic compounds) do not necessarily have to have an LC phase themselves. It is also possible for mesogenic compounds to exhibit LC phase behaviour only after mixing with other compounds and/or after polymerization. Typical mesogenic groups are, for example, rigid rod- or disc-shaped units. An overview of the terms and definitions used in connection with mesogenic or LC compounds is given in Pure Appl. Chem. 2001, 73(5), 888 and C. Tschierske, G. Pelzl, S. Diele, Angew. Chem. 2004, 116, 6340-6368.

The term “spacer group”, hereinafter also referred to as “Sp”, as used herein is known to the person skilled in the art and is described in the literature, see, for example, Pure Appl. Chem. 2001, 73(5), 888 and C. Tschierske, G. Pelzl, S. Diele, Angew. Chem. 2004, 116, 6340-6368. As used herein, the terms “spacer group” or “spacer” mean a flexible group, for example an alkylene group, which connects the mesogenic group and the polymerizable group(s) in a polymerizable mesogenic compound.

If in the formulae shown above and below a group R including any variations thereof such a R¹, R⁰ R¹¹, etc. or L denotes an alkyl radical and/or an alkoxy radical, this may be straight-chain or branched. It is preferably straight-chain, has 2, 3, 4, 5, 6 or 7 C atoms and accordingly preferably denotes ethyl, propyl, butyl, pentyl, hexyl, heptyl, ethoxy, propoxy, butoxy, pentoxy, hexyloxy or heptyloxy, furthermore methyl, octyl, nonyl, decyl, undecyl, dodecyl, tridecyl, tetradecyl, pentadecyl, methoxy, octyloxy, nonyloxy, decyloxy, undecyloxy, dodecyloxy, tridecyloxy or tetradecyloxy.

If in the formulae shown above and below a group R including any variations thereof such a R¹, R⁰ R¹¹, etc. or L denotes an alkyl radical and/or an alkoxy radical, this may be straight-chain or branched. It is preferably straight-chain, has 2, 3, 4, 5, 6 or 7 C atoms and accordingly preferably denotes ethyl, propyl, butyl, pentyl, hexyl, heptyl, ethoxy, propoxy, butoxy, pentoxy, hexyloxy or heptyloxy, furthermore methyl, octyl, nonyl, decyl, undecyl, dodecyl, tridecyl, tetradecyl, pentadecyl, methoxy, octyloxy, nonyloxy, decyloxy, undecyloxy, dodecyloxy, tridecyloxy or tetradecyloxy.

If in the formulae shown above and below a group R including any variations thereof such a R¹, R⁰ R¹¹, etc. or L denotes an alkyl radical wherein one or more CH₂ groups are replaced by S, this may be straight-chain or branched. It is preferably straight-chain, has 1, 2, 3, 4, 5, 6 or 7 C atoms and accordingly preferably denotes thiomethyl, thioethyl, thiopropyl, thiobutyl, thiopentyl, thiohexyl or thioheptyl.

Oxaalkyl preferably denotes straight-chain 2-oxapropyl (=methoxymethyl), 2-(=ethoxymethyl) or 3-oxabutyl (=2-methoxyethyl), 2-, 3- or 4-oxapentyl, 2-, 3-, 4- or 5-oxahexyl, 2-, 3-, 4-, 5- or 6-oxaheptyl, 2-, 3-, 4-, 5-, 6- or 7-oxaoctyl, 2-, 3-, 4-, 5-, 6-, 7- or 8-oxanonyl, 2-, 3-, 4-, 5-, 6-, 7-, 8- or 9-oxa-decyl.

If in the formulae shown above and below a group R including any variations thereof such a R¹, R⁰ R¹¹, etc. or L denotes an alkoxy or oxaalkyl group it may also contain one or more additional oxygen atoms, provided that oxygen atoms are not linked directly to one another.

In another preferred embodiment, one or more of R including any variations thereof such a R¹, R⁰ R¹¹, etc. or L are selected from the group consisting of

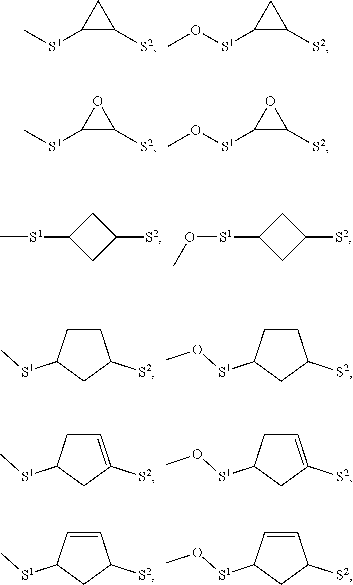

—S¹—F, —O—S¹—F, —O—S₁—O—S₂, wherein S¹ is C₁₋₁₂-alkylene or C₂₋₁₂-alkenylene and S² is H, C₁₋₁₂-alkyl or C₂₋₁₂-alkenyl, and very preferably are selected from the group consisting of

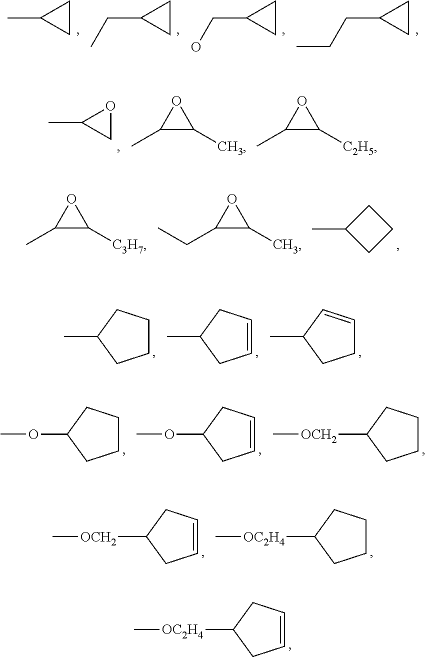

—OCH₂OCH₃, —O(CH₂)₂OCH₃, —O(CH₂)₃OCH₃, —O(CH₂)₄OCH₃, —O(CH₂)₂F, —O(CH₂)₃F and —O(CH₂)₄F.

If in the formulae shown above and below a group R including any variations thereof such a R¹, R⁰ R¹¹, etc. or L denotes an alkyl radical in which one CH₂ group has been replaced by —CH═CH—, this may be straight-chain or branched. It is preferably straight-chain and has 2 to 10 C atoms. Accordingly, it denotes, in particular, vinyl, prop-1- or -2-enyl, but-1-, -2- or -3-enyl, pent-1-, -2-, -3- or -4-enyl, hex-1-, -2-, -3-, -4- or -5-enyl, hept-1-, -2-, -3-, -4-, -5- or -6-enyl, oct-1-, -2-, -3-, -4-, -5-, -6- or -7-enyl, non-1-, -2-, -3-, -4-, -5-, -6-, -7- or -8-enyl, dec-1-, -2-, -3-, -4-, -5-, -6-, -7-, -8- or -9-enyl.

If in the formulae shown above and below a group R including any variations thereof such a R¹, R⁰ R¹¹, etc. or L denotes an alkyl or alkenyl radical which is at least monosubstituted by halogen, this radical is preferably straight-chain, and halogen is preferably F or CI. In the case of polysubstitution, halogen is preferably F. The resultant radicals also include perfluorinated radicals. In the case of monosubstitution, the fluorine or chlorine substituent may be in any desired position, but is preferably in the ω-position.

Halogen is preferably F or Cl, very preferably F.

The group —CR⁰═CR⁰⁰— is preferably —CH═CH—.
—OC—, —CO—, —C(═O)— and —C(O)— denote a carbonyl group, i.e.

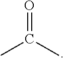

Preferred substituents L, are, for example, F, Cl, Br, I, —CN, —NO₂, —NCO, —NCS, —OCN, —SCN, —C(═O)N(R^{x})₂, —C(═O)Y¹, —C(═O)R^{x}, —N(R^{x})₂, straight-chain or branched alkyl, alkoxy, alkylcarbonyl, alkoxycarbonyl, alkylcarbonyloxy or alkoxycarbonyloxy each having 1 to 25 C atoms, in which one or more H atoms may optionally be replaced by F or Cl, optionally substituted silyl having 1 to 20 Si atoms, or optionally substituted aryl having 6 to 25, preferably 6 to 15, C atoms,
wherein R including any variations thereof such a R¹, R⁰ R¹¹, etc. denotes H, F, Cl, CN, or straight chain, branched or cyclic alkyl having 1 to 25 C atoms, wherein one or more non-adjacent CH₂-groups are optionally replaced by —O—, —S—, —CO—, —CO—O—, —O—CO—, —O—CO—O— in such a manner that O- and/or S-atoms are not directly connected with each other, and wherein one or more H atoms are each optionally replaced by F, Cl, P— or P—Sp—, and
Y¹ denotes halogen.

Particularly preferred substituents L are, for example, F, Cl, CN, NO₂, CH₃, C₂H₅, OCH₃, OC₂H₅, COCH₃, COC₂H₅, COOCH₃, COOC₂H₅, CF₃, OCF₃, OCHF₂, OC₂F₅, furthermore phenyl.

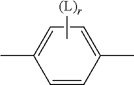

is preferably

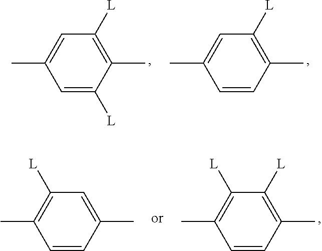

in which L has one of the meanings indicated above.

The polymerizable group P is a group which is suitable for a polymerization reaction, such as, for example, free-radical or ionic chain polymerization, polyaddition or polycondensation, or for a polymer-analogous reaction, for example addition or condensation onto a main polymer chain. Particular preference is given to groups for chain polymerization, in particular those containing a C═C double bond or —C≡C—triple bond, and groups which are suitable for polymerization with ring opening, such as, for example, oxetane or epoxide groups.

Preferred groups P are selected from the group consisting of CH₂═CW¹—CO—O—, CH₂═CW¹—CO—,

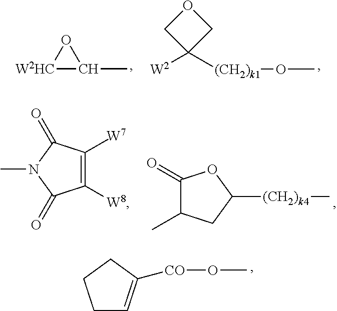

CH₂═CW²—(O)ₖ₃—, CW¹═CH—CO—(O)ₖ₃—, CW¹═CH—CO—NH—, CH₂═CW¹—CO—NH—, CH₃—CH═CH—O—, (CH₂═CH)₂CH—OCO—, (CH₂═CH—CH₂)₂CH—OCO—, (CH₂═CH)₂CH—O—, (CH₂═CH—CH₂)₂N—, (CH₂═CH—CH₂)₂N—CO—, HO—CW²W³—, HS—CW²W³—, HW²N—, HO—CW²W³—NH—, CH₂═CW¹—CO—NH—, CH₂═CH—(COO)ₖ₁-Phe-(O)ₖ₂—, CH₂═CH—(CO)ₖ₁-Phe-(O)ₖ₂—, Phe-CH═CH—, HOOC—, OCN— and W⁴W⁵W⁶Si—, in which W¹ denotes H, F, Cl, CN, CF₃, phenyl or alkyl having 1 to 5 C atoms, in particular H, F, Cl or CH₃, W² and W³ each, independently of one another, denote H or alkyl having 1 to 5 C atoms, in particular H, methyl, ethyl or n-propyl, W⁴, W⁶ and We each, independently of one another, denote Cl, oxaalkyl or oxacarbonylalkyl having 1 to 5 C atoms, W⁷ and W⁸ each, independently of one another, denote H, Cl or alkyl having 1 to 5 C atoms, Phe denotes 1,4-phenylene, which is optionally substituted by one or more radicals L as defined above which are other than P—Sp—, k₁, k₂ and k₃ each, independently of one another, denote 0 or 1, k₃ preferably denotes 1, and k₄ denotes an integer from 1 to 10.

Very preferred groups P are selected from the group consisting of CH₂═CW¹—CO—O—, CH₂═CW¹—CO—,

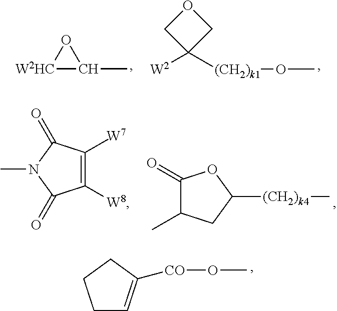

CH₂═CW²—O—, CH₂═CW²—,
CW¹═CH—CO—(O)ₖ₃—, CW¹═CH—CO—NH—, CH₂═CW¹—CO—NH—, (CH₂═CH)₂CH—OCO—, (CH₂═CH—CH₂)₂CH—OCO—, (CH₂═CH)₂CH—O—, (CH₂═CH—CH₂)₂N—, (CH₂═CH—CH₂)₂N—CO—, CH₂═CW¹—CO—NH—, CH₂═CH—(COO)ₖ₁-Phe-(O)ₖ₂—, CH₂═CH—(CO)ₖ₁-Phe-(O)ₖ₂—, Phe-CH═CH— and W⁴W⁵W⁶Si—, in which W¹ denotes H, F, Cl, CN, CF₃, phenyl or alkyl having 1 to 5 C atoms, in particular H, F, Cl or CH₃, W² and W³ each, independently of one another, denote H or alkyl having 1 to 5 C atoms, in particular H, methyl, ethyl or n-propyl, W⁴, W⁵ and W⁶ each, independently of one another, denote Cl, oxaalkyl or oxacarbonylalkyl having 1 to 5 C atoms, W⁷ and W⁸ each, independently of one another, denote H, Cl or alkyl having 1 to 5 C atoms, Phe denotes 1,4-phenylene, k₁, k₂ and k₃ each, independently of one another, denote 0 or 1, k₃ preferably denotes 1, and k₄ denotes an integer from 1 to 10.

Very particularly preferred groups P are selected from the group consisting of CH₂═CW—CO—O—, in particular CH₂═CH—CO—O—, CH₂═C(CH₃)—CO—O— and CH₂═CF—CO—O—, furthermore CH₂═CH—O—, (CH₂═CH)₂CH—O—CO—,

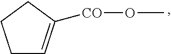

(CH₂═CH)₂CH—O—,

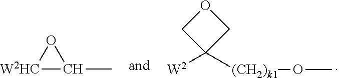

Further preferred polymerizable groups P are selected from the group consisting of vinyloxy, acrylate, methacrylate, fluoroacrylate, chloroacrylate, oxetane and epoxide, most preferably from acrylate and methacrylate.

Very preferably all polymerizable groups in the polymerizable compound have the same meaning.

If the spacer group Sp is different from a single bond, it is preferably of the formula Sp″-X″, so that the respective radical P-Sp- conforms to the formula P-Sp″-X″—, wherein
Sp″ denotes linear or branched alkylene having 1 to 20, preferably 1 to 12, C atoms, which is optionally mono- or polysubstituted by F, Cl, Br, I or CN and in which, in addition, one or more non-adjacent CH₂ groups may each be replaced, independently of one another, by —O—, —S—, —NH—, —N(R⁰)—, —Si(R⁰R⁰⁰)—, —CO—, —CO—O—, —O—CO—, —O—CO—O—, —S—CO—, —CO—S—, —N(R⁰⁰)—CO—O—, —O—CO—N(R⁰)—, —N(R⁰)—CO—N(R⁰⁰)—, —CH═CH— or —C═C— in such a way that O and/or S atoms are not linked directly to one another,
X″ denotes —O—, —S—, —CO—, —CO—O—, —O—CO—, —O—CO—O—, —CO—N(R⁰)—, —N(R⁰)—CO—, —N(R⁰)—CO—N(R⁰⁰)—, —OCH₂—, —CH₂O—, —SCH₂—, —CH₂S—, —CF₂O—, —OCF₂—, —CF₂S—, —SCF₂—, —CF₂CH₂—, —CH₂CF₂—, —CF₂CF₂—, —CH═N—, —N═CH—, —N═N—, —CH═CRO—, —CY₂═CY₃—, —C═C—, —CH═CH—CO—O—, —O—CO—CH═CH— or a single bond,
R⁰ and R⁰⁰ each, independently of one another, denote H or alkyl having 1 to 20 C atoms, and
Y² and Y³ each, independently of one another, denote H, F, Cl or CN.
X″ is preferably —O—, —S—, —CO—, —COO—, —OCO—, —O—COO—, —CO—NR⁰—, —NR⁰—CO—, —NR⁰—CO—NR⁰⁰— or a single bond.

Typical spacer groups Sp and -Sp″-X″— are, for example, —(CH₂)ₚ₁—, —(CH₂)ₚ₁—O—, —(CH₂)ₚ₁—O—CO—, —(CH₂)ₚ₁—CO—O—, —(CH₂)ₚ₁—O—CO—O—, —(CH₂CH₂O)_{q1}—CH₂CH₂—, —CH₂CH₂—S—CH₂CH₂—, —CH₂CH₂—NH—CH₂CH₂— or —(SiR⁰R⁰⁰—O)ₚ₁—, in which p1 is an integer from 1 to 12, q1 is an integer from 1 to 3, and R⁰ and R⁰⁰ have the meanings indicated above.

Particularly preferred groups Sp and -Sp″-X″— are —(CH₂)ₚ₁—, —(CH₂)ₚ₁—O—, —(CH₂)ₚ₁—O—CO—, —(CH₂)ₚ₁—CO—O—, —(CH₂)ₚ₁—O—CO—O—, in which p1 and q1 have the meanings indicated above.

Particularly preferred groups Sp″ are, in each case straight-chain, ethylene, propylene, butylene, pentylene, hexylene, heptylene, octylene, nonylene, decylene, undecylene, dodecylene, octadecylene, ethyleneoxyethylene, methyleneoxybutylene, ethylenethioethylene, ethylene-N-methyliminoethylene, 1-methylalkylene, ethenylene, propenylene and butenylene.

In a preferred embodiment of the invention the compounds of formula I and its subformulae contain a spacer group Sp that is substituted by one or more polymerizable groups P, so that the group Sp-P corresponds to Sp(P)ₛ, with s being ≥2 (branched polymerizable groups).

Preferred compounds of formula I according to this preferred embodiment are those wherein s is 2, i.e. compounds which contain a group Sp(P)₂. Very preferred compounds of formula I according to this preferred embodiment contain a group selected from the following formulae:

—X-alkyl-CHPP S1

—X-alkyl-CH(CH₂)ₐₐP)(CH₂)_{bb}P) S2

—X—N((CH₂)ₐₐP)((CH₂)_{bb}P) S3

—X-alkyl-CHP—CH₂—CH₂P S4

—X-alkyl-C(CH₂P)(CH₂P)—CₐₐH₂ₐₐ₊₁ S5

—X-alkyl-CHP—CH₂P S6

—X-alkyl-CPP—CₐₐH₂ₐₐ₊₁ S7

—X-alkyl-CHPCHP—CₐₐH₂ₐₐ₊₁ S8
in which P is as defined in formula I,
alkyl denotes a single bond or straight-chain or branched alkylene having 1 to 12 C atoms which is unsubstituted or mono- or polysubstituted by F, Cl or CN and in which one or more non-adjacent CH₂ groups may each, independently of one another, be replaced by —C(R⁰)═C(R⁰)—, —C═C—, —N(R⁰)—, —O—, —S—, —CO—, —CO—O—, —O—CO—, —O—CO—O— in such a way that O and/or S atoms are not linked directly to one another, where R⁰ has the meaning indicated above,
aa and bb each, independently of one another, denote 0, 1, 2, 3, 4, 5 or 6,
X has one of the meanings indicated for X″, and is preferably O, CO, SO₂, O—CO—, CO—O or a single bond.

Preferred spacer groups Sp(P)₂ are selected from formulae S1, S2 and S3.

Very preferred spacer groups Sp(P)₂ are selected from the following subformulae:

—CHPP S1a

—O—CHPP S1b

—CH₂—CHPP S1c

—OCH₂—CHPP S1d

—CH(CH₂—P)(CH₂—P) S2a

—OCH(CH₂—P)(CH₂—P) S2b

—CH₂—CH(CH₂—P)(CH₂—P) S2c

—OCH₂—CH(CH₂—P)(CH₂—P) S2d

—CO—NH((CH₂)₂P)((CH₂)₂P) S3a

P is preferably selected from the group consisting of vinyloxy, acrylate, methacrylate, fluoroacrylate, chloroacrylate, oxetane and epoxide, very preferably from acrylate and methacrylate, most preferably from methacrylate.

Further preferably all polymerizable groups P that are present in the same compound have the same meaning, and very preferably denote acrylate or methacrylate, most preferably acrylate.

The term “film” as used herein includes rigid or flexible, self-supporting or free-standing films with mechanical stability, as well as coatings or layers on a supporting substrate or between two substrates. “Thin film” means a film having a thickness in the nanometer or micrometer range, preferably at least 10 nm, very preferably at least 100 nm, and preferably not more than 100 μm, very preferably not more than 10 μm.

Throughout the application, the term “aryl and heteroaryl groups” encompass groups, which can be monocyclic or polycyclic, i.e. they can have one ring (such as, for example, phenyl) or two or more rings, which may also be fused (such as, for example, naphthyl) or covalently linked (such as, for example, biphenyl), or contain a combination of fused and linked rings. Heteroaryl groups contain one or more heteroatoms, preferably selected from O, N, S and Se. Particular preference is given to mono-, bi- or tricyclic aryl groups having 6 to 25 C atoms and mono-, bi- or tricyclic heteroaryl groups having 2 to 25 C atoms, which optionally contain fused rings, and which are optionally substituted. Preference is furthermore given to 5, 6 or 7-membered aryl and heteroaryl groups, in which, in addition, one or more CH groups may be replaced by N, S or O in such a way that O atoms and/or S atoms are not linked directly to one another. Preferred aryl groups are, for example, phenyl, biphenyl, terphenyl, [1,1′:3′, 1″]¬¬terphenyl-2′-yl, naphthyl, anthracene, binaphthyl, phenanthrene, pyrene, dihydropyrene, chrysene, perylene, tetracene, pentacene, benzopyrene, fluorene, indene, indenofluorene, spirobifluorene, more preferably 1,4-phenylene, 4,4′-biphenylene, 1, 4-tephenylene.

Preferred heteroaryl groups are, for example, 5 membered rings, such as pyrrole, pyrazole, imidazole, 1,2,3-triazole, 1,2,4-triazole, tetrazole, furan, thiophene, selenophene, oxazole, isoxazole, 1,2 thiazole, 1,3-thiazole, 1,2,3-oxadiazole, 1,2,4 oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 6 membered rings, such as pyridine, pyridazine, pyrimidine, pyrazine, 1,3,5-triazine, 1,2,4-triazine, 1,2,3-triazine, 1,2,4,5-tetrazine, 1,2,3,4-tetrazine, 1,2,3,5-tetrazine, or condensed groups, such as indole, iso-indole, indolizine, indazole, benzimidazole, benzotriazole, purine, naphthimidazole, phenanthrimidazole, pyridimidazole, pyrazinimidazole, quinoxalinimidazole, benzoxazole, naphthoxazole, anthroxazole, phen-anthroxazole, isoxazole, benzothiazole, benzofuran, isobenzofuran, dibenzofuran, quinoline, isoquinoline, pteridine, benzo-5,6-quinoline, benzo-6,7-quinoline, benzo-7,8-quinoline, benzoisoquinoline, acridine, phenothiazine, phenoxazine, benzopyridazine, benzopyrimidine, quinoxaline, phenazine, naphthyridine, azacarbazole, benzocarboline, phenanthridine, phenanthroline, thieno[2,3b]thiophene, thieno[3,2b]-thiophene, dithienothiophene, isobenzothiophene, dibenzothiophene, benzothiadiazothiophene, or combinations of these groups. The heteroaryl groups may also be substituted by alkyl, alkoxy, thioalkyl, fluorine, fluoroalkyl or further aryl or heteroaryl groups.

The term “chiral” in general is used to describe an object that is non-superimposable on its mirror image.

“Achiral” (non-chiral) objects are objects that are identical to their mirror image.

The terms “chiral nematic” and “cholesteric” are used synonymously in this application, unless explicitly stated otherwise.

The reflection wavelength λ is given by the pitch p of the cholesteric helix and the mean birefringence n of the cholesteric liquid crystal in accordance with the following equation:

λ=n·p

A CLC medium can be prepared, for example, by doping a nematic LC medium with a chiral dopant having a high twisting power. The pitch p of the induced cholesteric helix is then given by the concentration c and the helical twisting power HTP of the chiral dopant in accordance with the following equation:

p=(HTP c)⁻¹

It is also possible to use two or more dopants, for example in order to com-pensate for the temperature dependence of the HTP of the individual dopants and thus to achieve low temperature dependence of the helix pitch and the reflection wavelength of the CLC medium. For the total HTP (HTPₜₒₜₐₗ) holds then approximately the following equation:

HTPₜₒₜₐₗ=ΣᵢcᵢHTPᵢ

wherein cᵢ is the concentration of each individual dopant and HTPᵢ is the helical twisting power of each individual dopant.

Above and below,

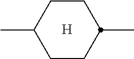

denotes a trans-1,4-cyclohexylene ring, and

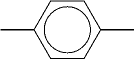

denotes a 1,4-phenylene ring.

Visible light is electromagnetic radiation that has wavelength in a range from about 400 nm to about 740 nm. Ultraviolet (UV) light is electromagnetic radiation with a wavelength in a range from about 200 nm to about 450 nm.

The Irradiance (Eₑ) or radiation power is defined as the power of electromagnetic radiation (dθ) per unit area (dA) incident on a surface:

Eₑ=dθ/dA.

The radiant exposure or radiation dose (Hₑ), is as the irradiance or radiation power (Eₑ) per time (t):

Hₑ=Eₑ·t.

All temperatures, such as, for example, the melting point T(C,N) or T(C,S), the transition from the smectic (S) to the nematic (N) phase T(S,N) and the clearing point T(N,I) of the liquid crystals, are quoted in degrees Celsius. All temperature differences are quoted in differential degrees.

The term “clearing point” means the temperature at which the transition between the mesophase with the highest temperature range and the isotropic phase occurs.

The term “director” is known in prior art and means the preferred orientation direction of the long molecular axes (in case of calamitic compounds) or short molecular axes (in case of discotic compounds) of the liquid-crystalline or RM molecules. In case of uniaxial ordering of such anisotropic molecules, the director is the axis of anisotropy.

The term “alignment” or “orientation” relates to alignment (orientational ordering) of anisotropic units of material such as small molecules or fragments of big molecules in a common direction named “alignment direction”. In an aligned layer of liquid-crystalline or RM material the liquid-crystalline director coincides with the alignment direction so that the alignment direction corresponds to the direction of the anisotropy axis of the material.

The terms “uniform orientation” or “uniform alignment” of an liquid-crystalline or RM material, for example in a layer of the material, mean that the long molecular axes (in case of calamitic compounds) or the short molecular axes (in case of discotic compounds) of the liquid-crystalline or RM molecules are oriented substantially in the same direction. In other words, the lines of liquid-crystalline director are parallel.

The term “homeotropic structure” or “homeotropic orientation” refers to a film wherein the optical axis is substantially perpendicular to the film plane.

The term “planar structure” or “planar orientation” refers to a film wherein the optical axis is substantially parallel to the film plane.

The term “A plate” refers to an optical retarder utilizing a layer of uniaxially birefringent material with its extraordinary axis oriented parallel to the plane of the layer.

The term “C plate” refers to an optical retarder utilizing a layer of uniaxially birefringent material with its extraordinary axis oriented perpendicular to the plane of the layer.

In A/C-plates comprising optically uniaxial birefringent liquid crystal material with uniform orientation, the optical axis of the film is given by the direction of the extraordinary axis. An A (or C) plate comprising optically uniaxial birefringent material with positive birefringence is also referred to as “positive A (or C) plate” or “+A (or +C) plate”.

An A (or C) plate comprising a film of optically uniaxial birefringent material with negative birefringence, such as discotic anisotropic materials is also referred to as “negative A (or C) plate” or “−A (or C) plate” depending on the orientation of the discotic materials. A film made from a cholesteric calamitic material with a reflection band in the UV part of the spectrum also has the optics of a negative C plate.

The birefringence An is defined as follows

Δn=nₑ−nₒ

wherein nₑ is the extraordinary refractive index and nₒ is the ordinary refractive index, and the average effective refractive index n_{av.} is given by the following equation:

n_{av.}=((2nₒ²+nₑ²)/3)^{1/2}

The average effective refractive index n_{av.} and the ordinary refractive index no can be measured using an Abbe refractometer. Δn can then be calculated from the above equations.

Unless the context clearly indicates otherwise, as used herein plural forms of the terms herein are to be construed as including the singular form and vice versa.

All physical properties have been and are determined according to “Merck Liquid Crystals, Physical Properties of Liquid Crystals”, Status Nov. 1997, Merck KGaA, Germany and are given for a temperature of 20° C., unless explicitly stated otherwise. The optical anisotropy (Δn) is determined at a wavelength of 589.3 nm

In a group

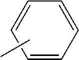

the single bond shown between the two ring atoms can be attached to any free position of the benzene ring. —OC—, —CO—, —C(═O)— and —C(O)— denote a carbonyl group, i.e.

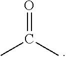

Above and below, percentages are percent by weight unless stated otherwise. All temperatures are given in degrees Celsius. m.p. denotes melting point, cl.p. denotes clearing point, T_{g} denotes glass transition temperature. Furthermore, C=crystalline state, N=nematic phase, S=smectic phase and I=isotropic phase. The data between these symbols represent the transition temperatures. An denotes the optical anisotropy or birefringence (Δn=nₑ−nₒ, where nₒ denotes the refractive index perpendicular to the longitudinal molecular axes and ne denotes the refractive index parallel thereto), measured at 550 nm and 20° C. The optical and electro optical data are measured at 20° C., unless expressly stated otherwise. “Clearing point” and “clearing temperature” mean the temperature of the transition from an LC phase into the isotropic phase.

Unless stated otherwise, the percentages of solid components in an RM mixture or RM formulation as described above and below refer to the total amount of solids in the mixture or formulation, i.e. without any solvents.

Unless stated otherwise, all optical, electro optical properties and physical parameters like birefringence, permittivity, electrical conductivity, electrical resistivity and sheet resistance, refer to a temperature of 20° C.

Unless the context clearly indicates otherwise, as used herein plural forms of the terms herein are to be construed as including the singular form and vice versa.

Throughout the description and claims of this specification, the words “comprise” and “contain” and variations of the words, for example “comprising” and “comprises”, mean “including but not limited to”, and are not intended to (and do not) exclude other components.

It will be appreciated that variations to the embodiments of the invention can be made while still falling within the scope of the invention. Each feature disclosed in this specification, unless stated otherwise, may be replaced by alternative features serving the same, equivalent or similar purpose. Thus, unless stated otherwise, each feature disclosed is one example only of a generic series of equivalent or similar features.

All of the features disclosed in this specification may be combined in any combination, except combinations where at least some of such features and/or steps are mutually exclusive. In particular, the preferred features of the invention are applicable to all aspects of the invention and may be used in any combination. Likewise, features described in non-essential combinations may be used separately (not in combination).

A “polymer network” is a network in which all polymer chains are interconnected to form a single macroscopic entity by many crosslinks.

The polymer network can occur in the following types:
A graft polymer molecule is a branched polymer molecule in which one or more the side chains are different, structurally or configurationally, from the main chain.
A star polymer molecule is a branched polymer molecule in which a single branch point gives rise to multiple linear chains or arms. If the arms are identical, the star polymer molecule is said to be regular. If adjacent arms are composed of different repeating subunits, the star polymer molecule is said to be variegated.
A comb polymer molecule consists of a main chain with two or more three-way branch points and linear side chains. If the arms are identical the comb polymer molecule is said to be regular.
A brush polymer molecule consists of a main chain with linear, unbranched side chains and where one or more of the branch points has four-way functionality or larger.

### DETAILED DESCRIPTION

In the compounds of formula I and its subformulae as described above and below, P is preferably selected from the group consisting of vinyloxy, acrylate, methacrylate, fluoroacrylate, chloroacrylate, oxetane and epoxide, very preferably from acrylate and methacrylate, most preferably acrylate.

Further preferred are compounds of formula I and its subformulae as described above and below, wherein all polymerizable groups P that are present in the compound have the same meaning, and very preferably denote acrylate or methacrylate, most preferably acrylate.

Further preferred are compounds of formula I and its subformulae as described above and below, which contain one, two, three or four groups P-Sp, very preferably two or three groups P-Sp.

Further preferred are compounds of formula I and its subformulae as described above and below, wherein R¹¹ is P-Sp-.

Further preferred are compounds of formula I and its subformulae as described above and below, wherein Sp denotes a single bond or —(CH₂)ₚ₁—, —O—(CH₂)ₚ₁—, —O—CO—(CH₂)ₚ₁, or —CO—O—(CH₂)ₚ₁, wherein p1 is 2, 3, 4, 5 or 6, and, if Sp is —O—(CH₂)ₚ₁—, —O—CO—(CH₂)ₚ₁ or —CO—O—(CH₂)ₚ₁ the O-atom or CO-group, respectively, is linked to the benzene ring.

Further preferred are compounds of formula I and its subformulae as described above and below, wherein at least one group Sp is a single bond.

Further preferred are compounds of formula I and its subformulae as described above and below, wherein at least one group Sp is a single bond and at least one group Sp is different from a single bond.

Further preferred are compounds of formula I and its subformulae as described above and below, wherein at least one group Sp is different from a single bond, and is selected from —(CH₂)ₚ₁—, —O—(CH₂)ₚ₁—, —O—CO—(CH₂)ₚ₁, or —CO—O—(CH₂)ₚ₁, wherein p1 is 2, 3, 4, 5 or 6, and, if Sp is —O—(CH₂)ₚ₁—, —O—CO—(CH₂)ₚ₁ or —CO—O—(CH₂)ₚ₁ the O-atom or CO-group, respectively, is linked to the benzene ring.

Very preferred are compounds of formula I and its subformulae as described above and below, wherein at least one group Sp is different from a single bond, and is selected from —(CH₂)₂—, —(CH₂)₃—, —(CH₂)₄—, —O—(CH₂)₂—, —O—(CH₂)₃—, —O—CO—(CH₂)₂ and —CO—O—(CH)₂—, wherein the O atom or the CO group is attached to the benzene ring.

Preferably one or more of the rings A, B, D and/or E in formula I are selected from the group consisting of 1,4-phenylene, 1,3-phenylene, naphthalene-1,4-diyl, naphthalene-2,6-diyl, phenanthrene-2,7-diyl, 9,10-dihydro-phenanthrene-2,7-diyl, anthracene-2,7-diyl, anthracene-9,10-diyl, fluorene-2,7-diyl, dibenzothiophene-2,7-diyl, dibenzofuran-2,7-diyl, benzo[1,2-b:4,5-b′]dithiophene-2,5-diyl, indole-4,7-diyl, benzothiophene-4,7-diyl, coumarine, flavone, where, in addition, one or more CH groups in these groups may be replaced by N, cyclohexane-1,4-diyl, in which, in addition, one or more non-adjacent CH₂ groups may be replaced by O and/or S, 1,4-cyclohexenylene, bicycle[1.1.1]pentane-1,3-diyl, bicyclo[2.2.2]octane-1,4-diyl, spiro[3.3]heptane-2,6-diyl, piperidine-1,4-diyl, decahydronaphthalene-2,6-diyl, 1,2,3,4-tetrahydronaphthalene-2,6-diyl, indane-2,5-diyl or octahydro-4,7-methanoindane-2,5-diyl, all of which are optionally substituted by one or more groups L or P-Sp-

Very preferably one or more of rings A, B, D and/or E in formula I are selected from the group consisting of benzene-1,4-diyl, naphthalene-1,4-diyl, naphthalene 2,6-diyl, phenanthrene-2,7-diyl, anthracene-9,10-diyl, fluoren-2,7-diyl, dibenzofuran-2,7-diyl, dibenzothiophene-2,7-diyl, benzo[1,2-b:4,5-b′]dithiophene-2,5-diyl, indole-4,7-diyl, benzothiophene-4,7-diyl, all of which are optionally substituted by one or more groups L or P-Sp. In these rings A, B, D and E naphthalene is preferably naphthalene-2,6-diyl or naphthalene 1,4-diyl and anthracene is preferably anthracene-9,10-diyl.

Ring C in formula I is preferably selected from the group consisting of benzene-1,4-diyl, naphthalene-1,4-diyl, anthracene-9,10-diyl, fluoren-2,7-diyl, dibenzofuran-2,7-diyl, dibenzothiophene-2,7-diyl, benzo[1,2-b:4,5-b′]dithiophene-2,5-diyl, indole-4,7-diyl, benzothiophene-4,7-diyl, very preferably benzene-1,4-diyl, naphthalene-1,4-diyl or anthracene-9,10-diyl, all of which are optionally substituted by one or more groups L or P-Sp.

If ring C is a benzene ring, it is preferably mono- or disubstituted by L.

More preferably one, two, three, four or more of rings A, B, D and/or E in formula I are selected from the group consisting of

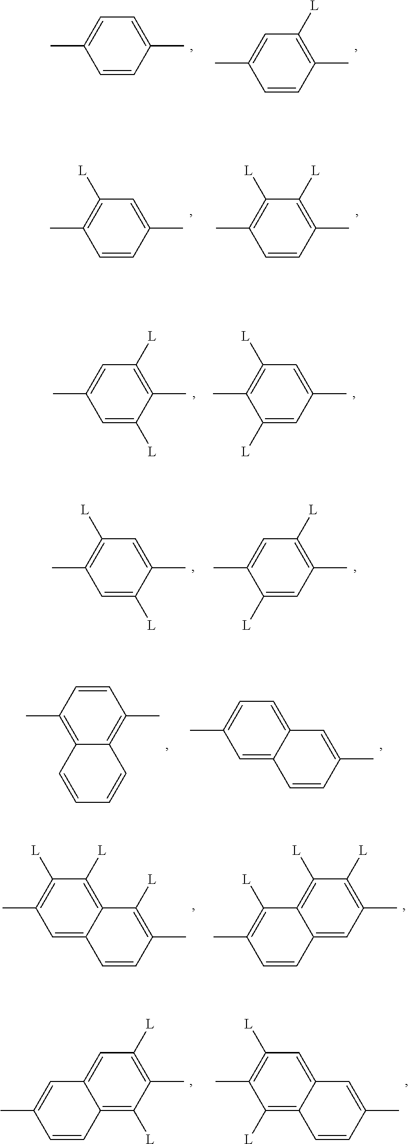

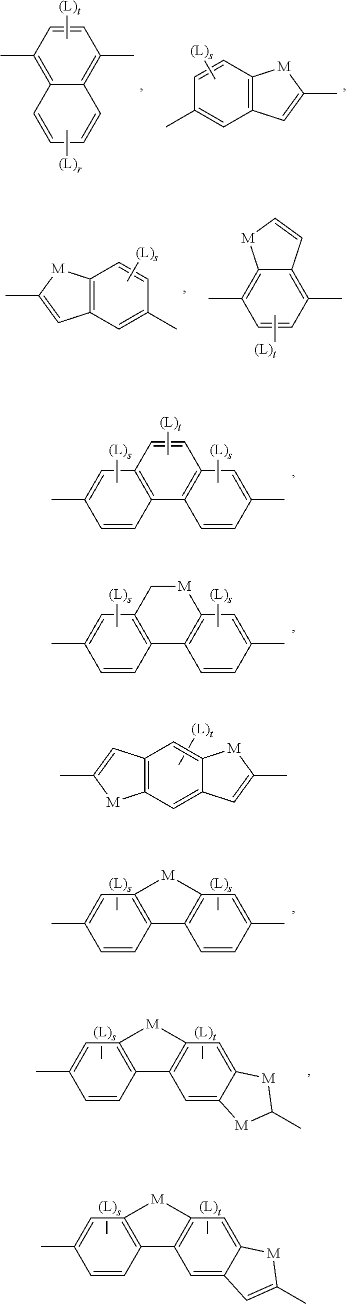

wherein
L each, independently of one another, denotes alkyl, alkoxy or thioalkyl having 1 to 6 C atoms, P-Sp-, —CN, F, Cl, OCF₃, CF₃, CH₂F, CHF₂, or two substituents L that are connected to directly adjacent C atoms may also form a cyclic group with 5, 6, 7 or 8 C atoms,
r denotes 0, 1, 2 or 3,
S denotes 0, 1 or 2,
t denotes 0, 1 or 2,
M denotes CH₂, C(CH₃)₂, CHF, CF₂, NH, S or O.

More preferably one or both of rings B and/or D in formula I are selected from the group consisting of

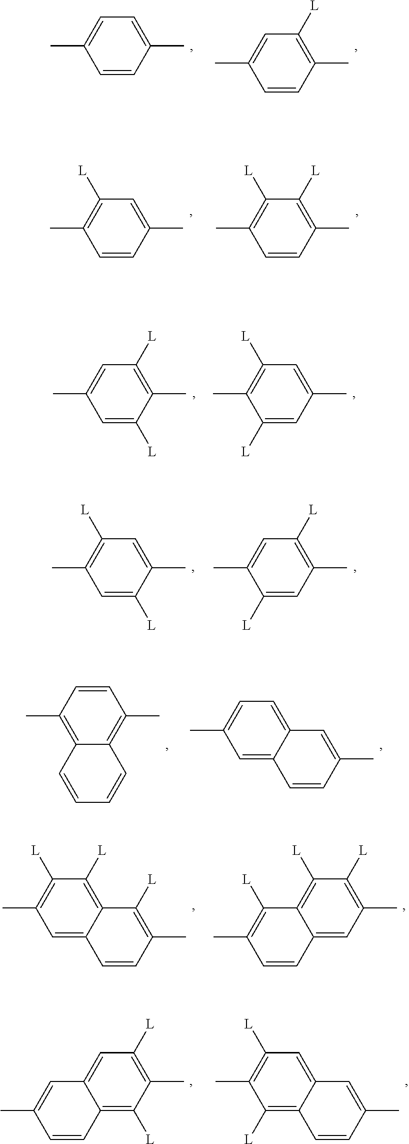

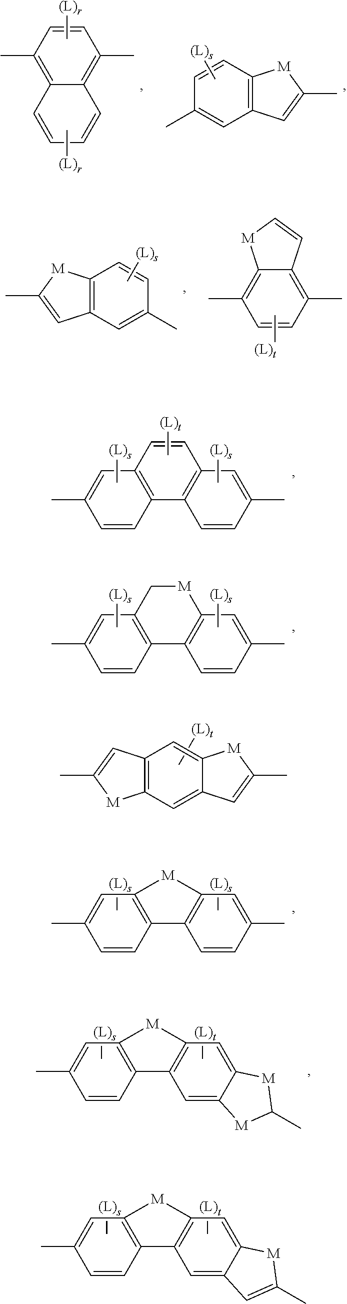

wherein
L each, independently of one another, denotes alkyl, alkoxy or thioalkyl having 1 to 6 C atoms, P-Sp-, —CN, F, Cl, OCF₃, CF₃, CH₂F, CHF₂, or two substituents L that are connected to directly adjacent C atoms may also form a cyclic group with 5, 6, 7 or 8 C atoms,
r denotes 0, 1, 2, or 3,
S denotes 0, 1 or 2,
t denotes 0, 1 or 2,
M denotes CH₂, C(CH₃)₂, CHF, CF₂, NH, S or O.

Especially preferred are compounds of formula I wherein one or both rings B and/or D denote a 2,6-naphthalene radical or a 1,4-naphthalene radical.

Preferably ring C in formula I is selected from the group consisting of

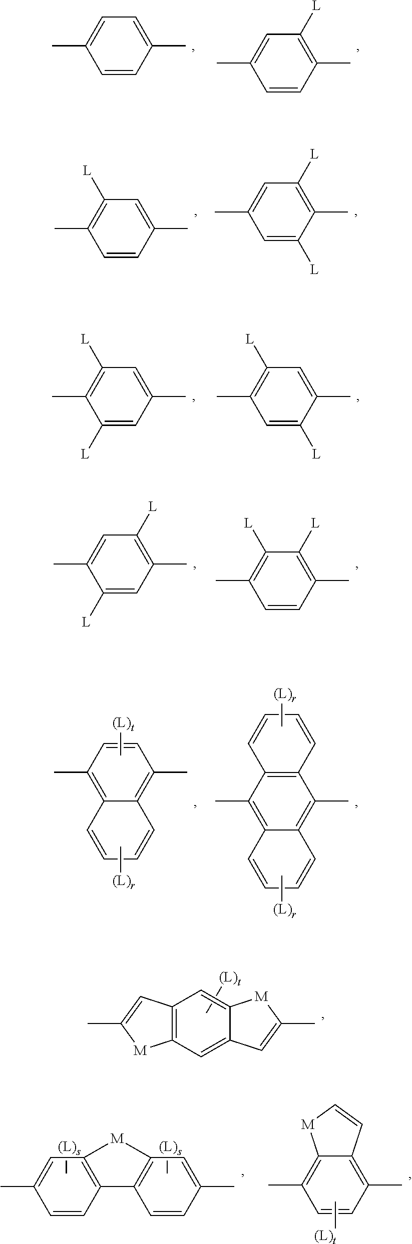

wherein
L each, independently of one another, denotes P-Sp-, F, —CN, alkyl, alkoxy or thioalkyl having 1 to 6, preferably 1 to 3, more preferably 1 or 2 C atoms,
r denotes 0, 1, 2 or 3,
S denotes 0, 1, 2 or 3,
t denotes 0, 1 or 2,
M denotes CH₂, C(CH₃)₂, CHF, CF₂, NH, S or O, and
M¹ denotes S, O or NH.

Very preferably ring C in formula I is selected from the group consisting of

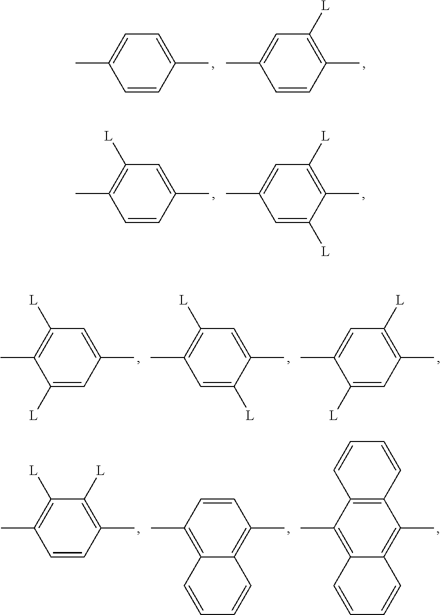

wherein L, independently of one another, denotes P-Sp-, F, —CN, alkyl, alkoxy or thioalkyl having 1 to 6, preferably 1 to 3, more preferably 1 or 2 C atoms.

Further preferred are compounds of formula I, especially those wherein n=m=0, wherein the rings B, C and D form a group

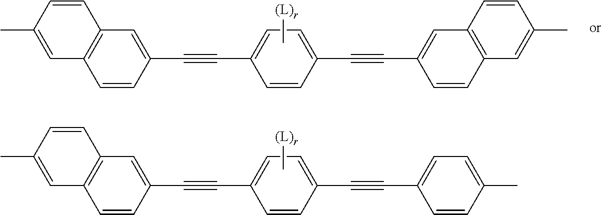

wherein
L each, independently of one another, denotes P-Sp-, F, —CN, alkyl, alkoxy or thioalkyl having 1 to 6, preferably 1 to 3, more preferably 1 or 2 C atoms, and
r is 0, 1, 2 or 3 preferably 1 or 2.

Further preferred are compounds of formula I, especially those wherein n=m=0, wherein the rings B, C and D form a group

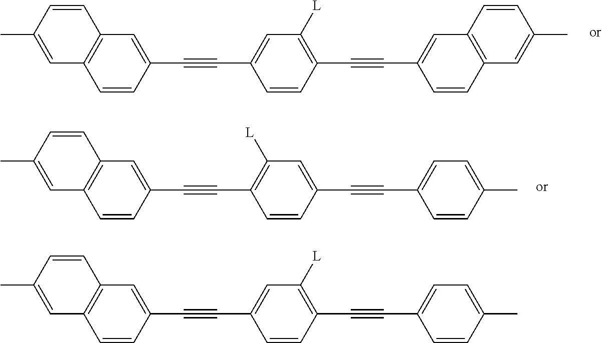

wherein L is methyl, ethyl, methoxy, ethoxy, thiomethyl or thioethyl, preferably ethyl.

Preferred compounds of formula I are selected from the following subformulae,

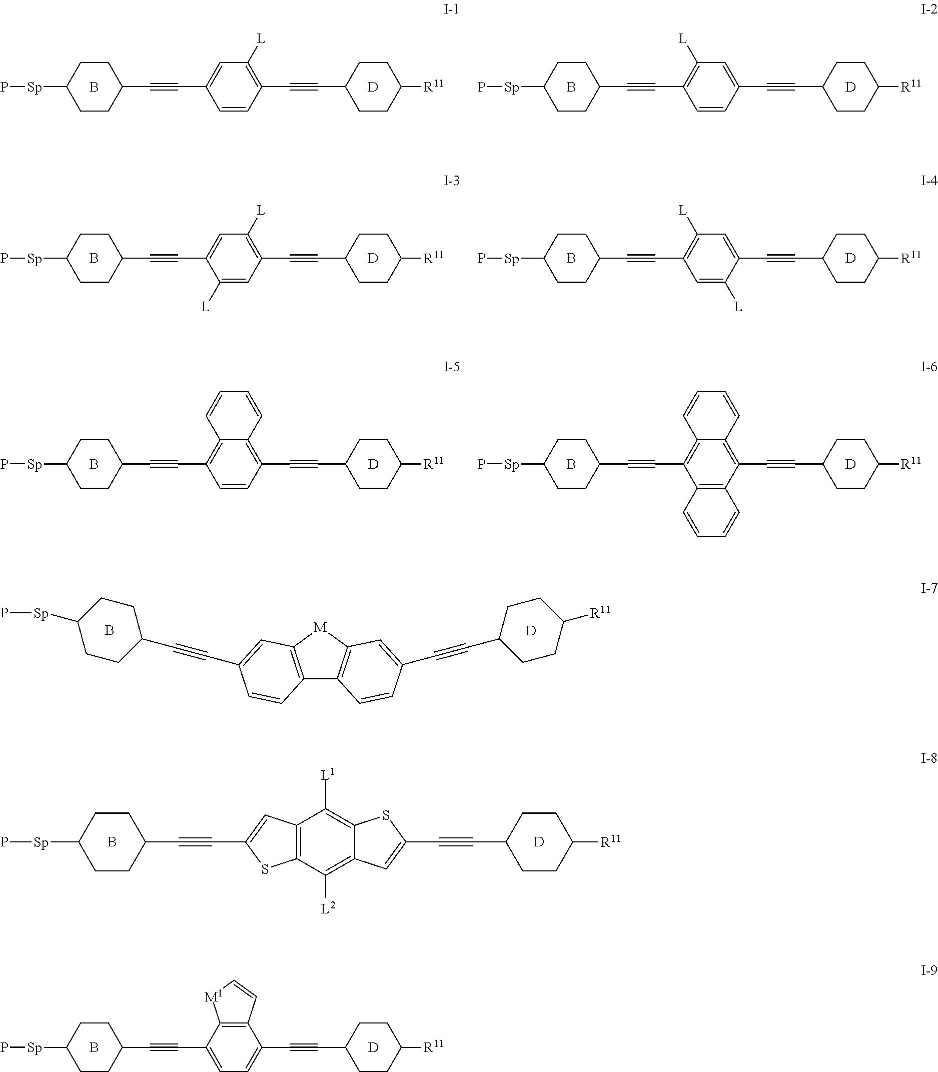

wherein
R¹¹, rings B and D, P and Sp have one of the meanings in formula I or one of their preferred meanings as given above and
wherein
L each, independently of one another, denotes P-Sp-, F, CN, alkyl, alkoxy or thioalkyl having 1 to 6, preferably 1 to 3, more preferably 1 or 2 C atoms,
L¹ and L² each, independently of one another, denote H or L,
M is S, O, NH, CH₂ or, C(CH₃)₂,
M¹ is NH or S.

Very preferred compounds of formula I are selected from the following subformulae,

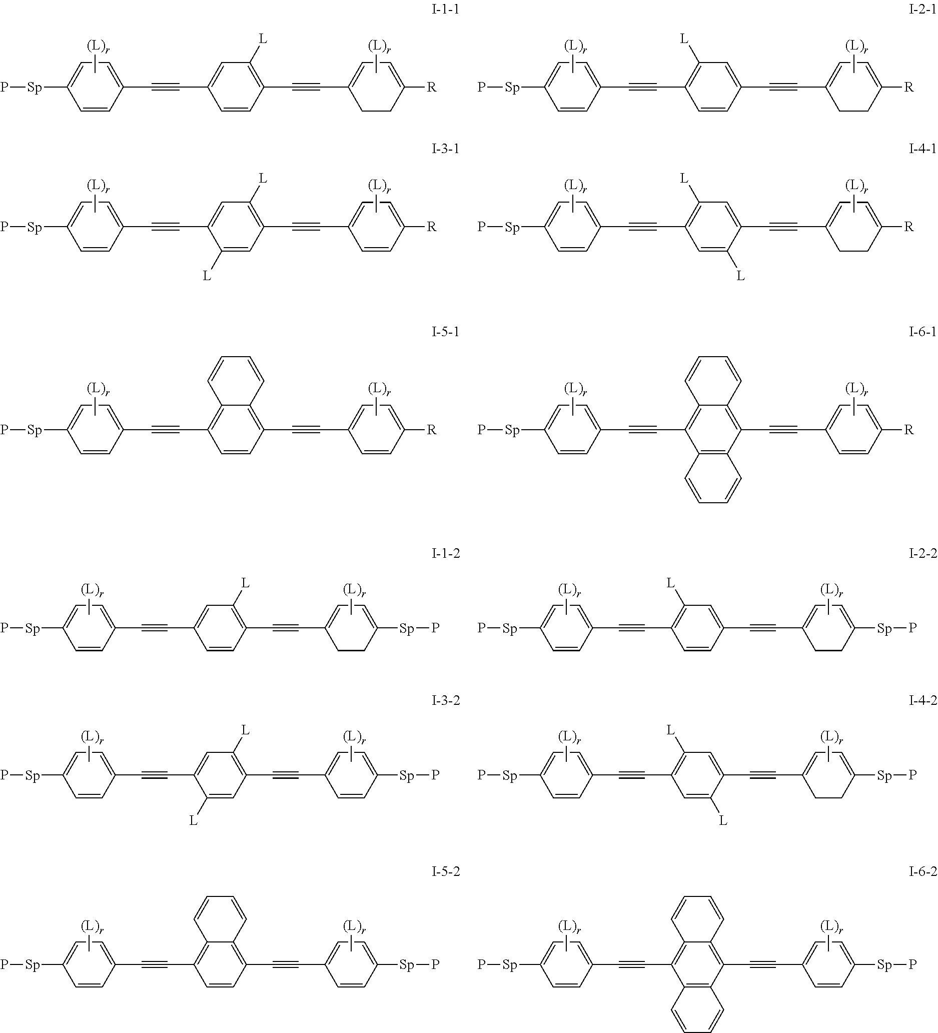

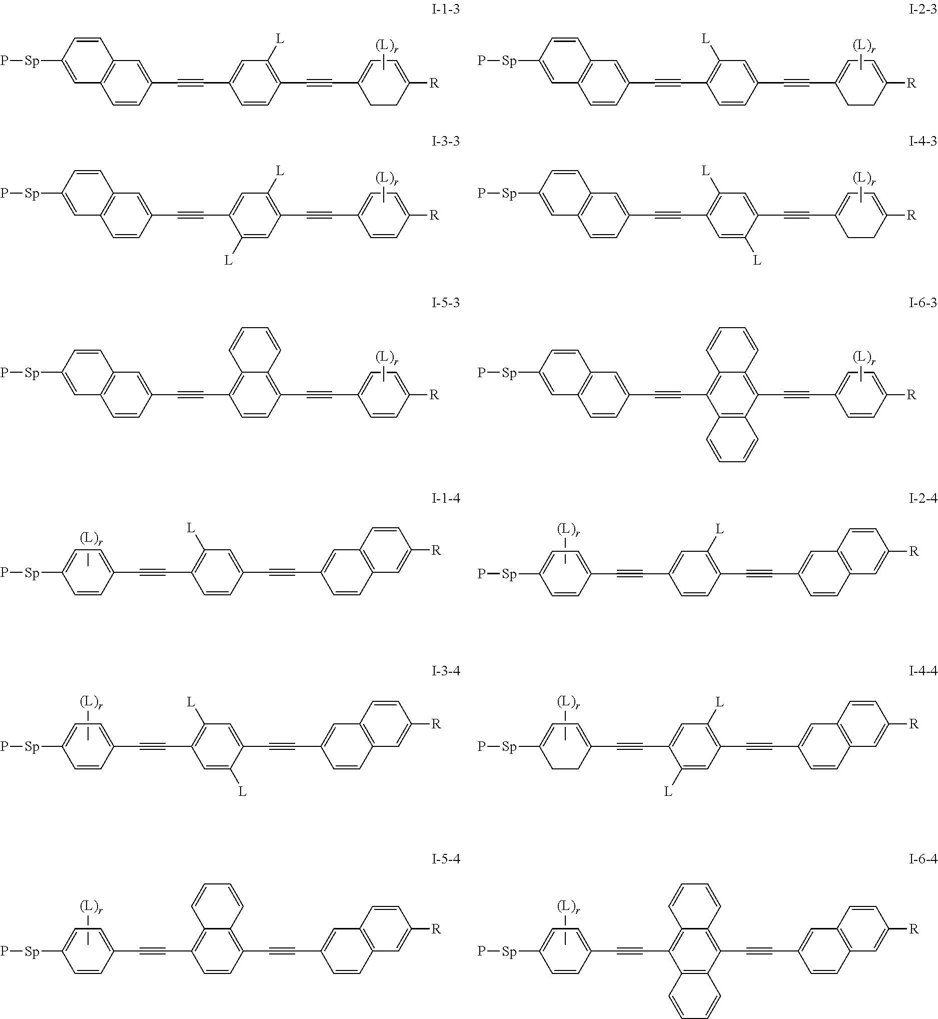

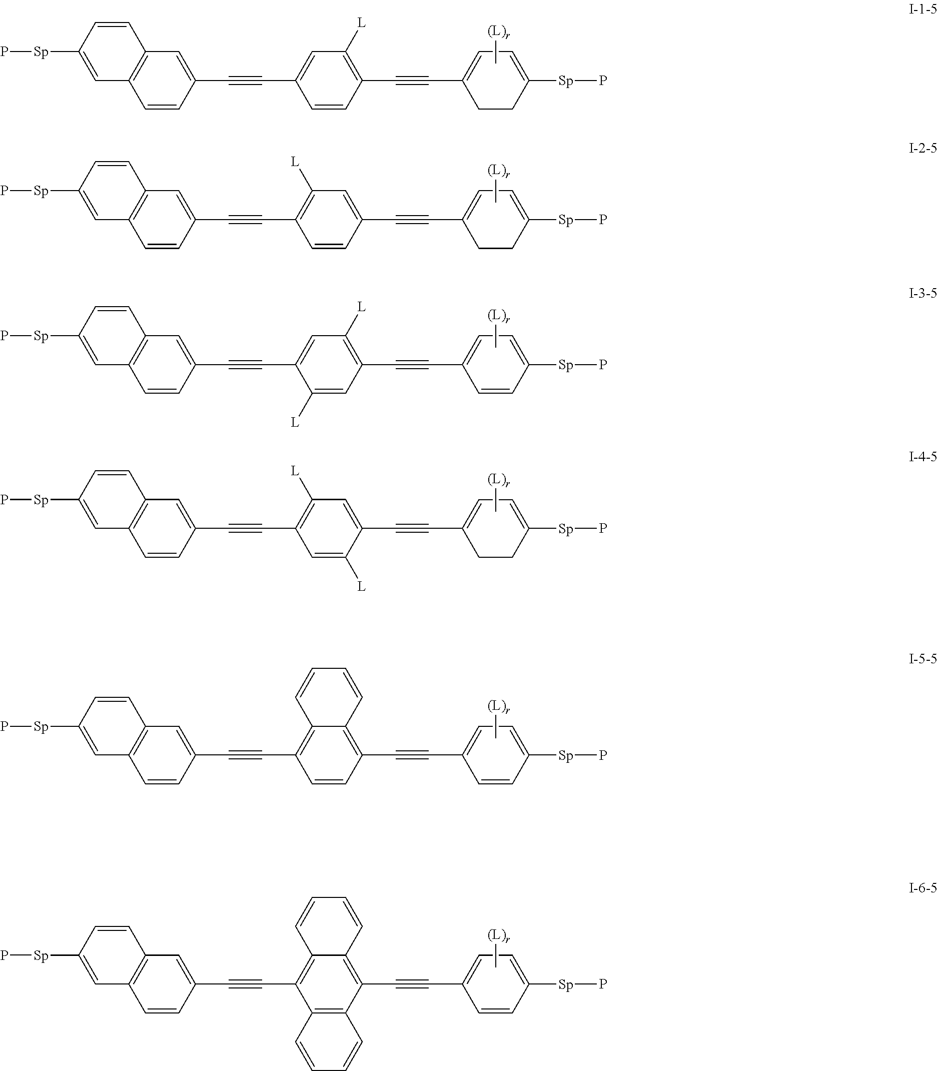

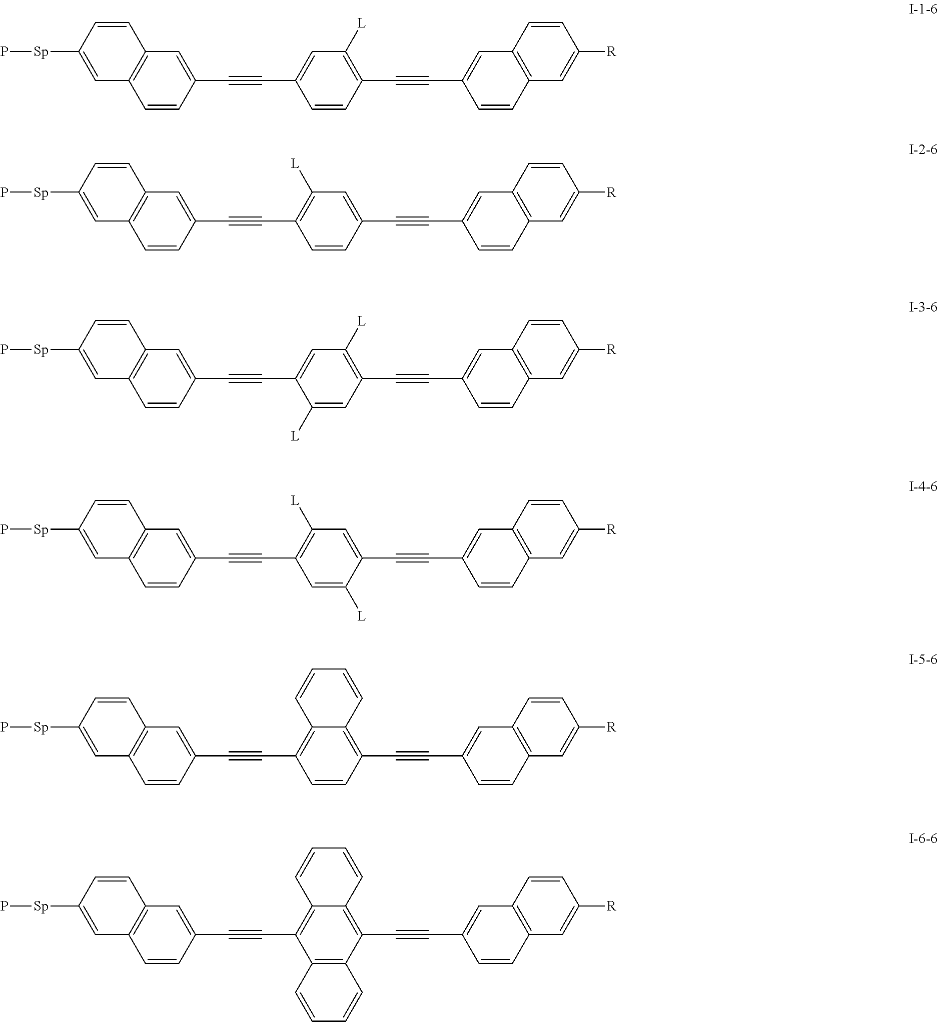

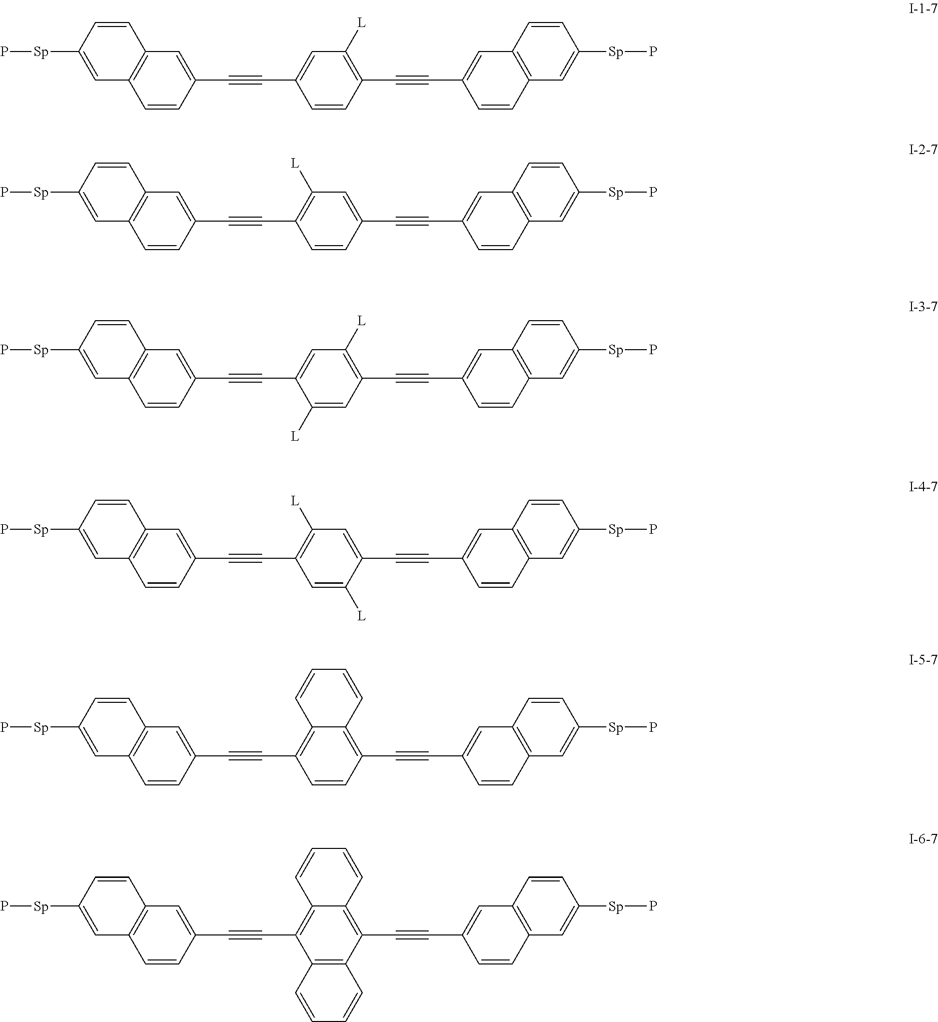

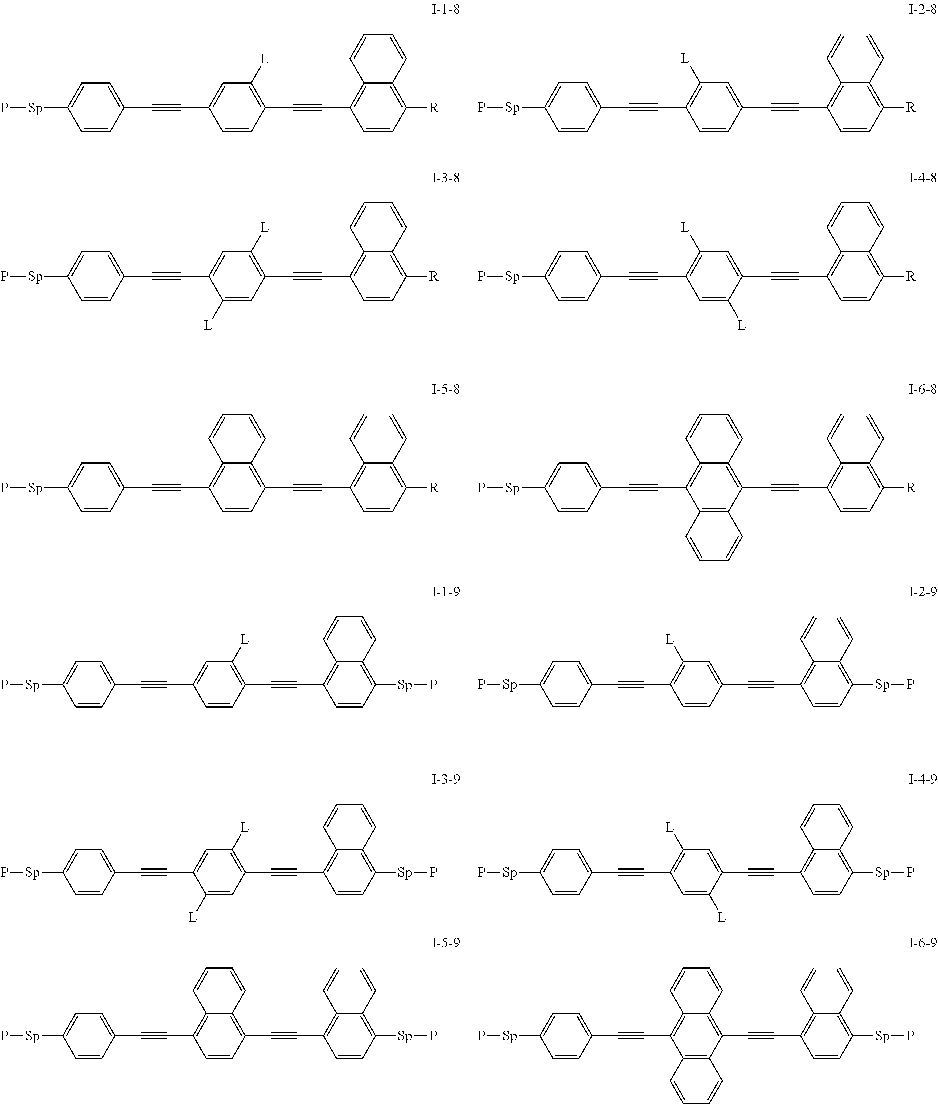

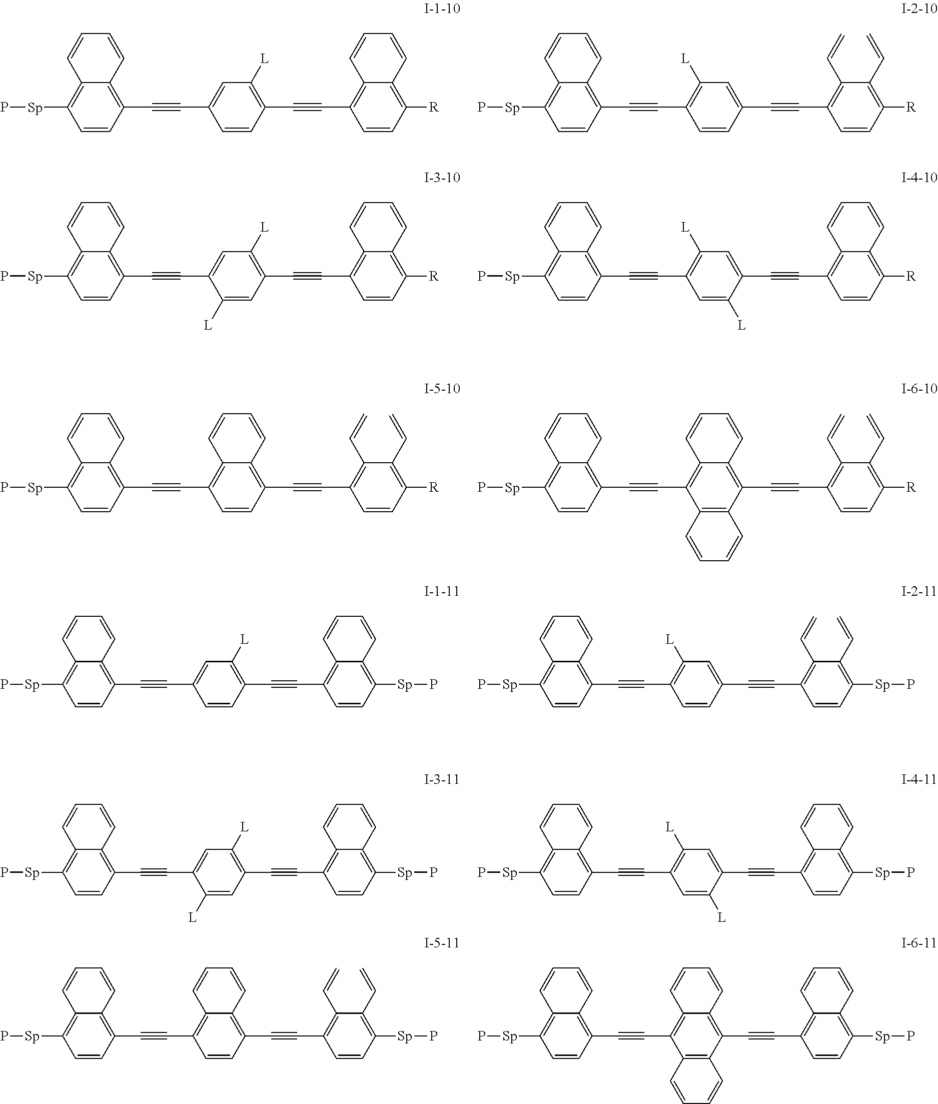

P is a polymerisable group,
Sp is a spacer group or a single bond,
R is alkyl, alkoxy, thioalkyl, alkylcarbonyl, alkoxycarbonyl, alkylcarbonyloxy or alkoxycarbonyloxy preferably with 1 to 15 C atoms which is more preferably optionally fluorinated,
L P-Sp- which is preferably acrylate, F, Cl, —CN, or straight chain, branched or cyclic alkyl having 1 to 25 C atoms, wherein one or more non-adjacent CH₂-groups are optionally replaced by —O—, —S—, —CO—, —CO—O—, —O—CO—, —O—CO—O—, CR⁰═CR⁰⁰—, —C≡C—,

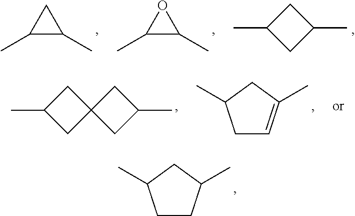

in such a manner that O- and/or S-atoms are not directly connected with each other, and wherein one or more H atoms are each optionally replaced by P-Sp-, F or Cl, and two substituents L that are connected to directly adjacent C atoms may also form a cyclic group with 5, 6, 7 or 8 C atoms,
r denotes 0, 1, 2, 3 or 4, preferably 0, 1 or 2.

Further preferred are compounds of formula I-1-2, I-2-2, I-3-2, I-4-2, I-5-2, I-6-2, I-1-5, I-2-5, I-3-5, I-4-5, I-5-5, I-6-5, I-1-7, I-2-7, I-3-7, I-4-7, I-5-7, I-6-7, I-1-9, I-2-9, I-3-9, I-4-9, I-5-9, I-6-9, I-1-11, I-2-11, I-3-11, I-4-11, I-5-11 and I-6-11 wherein one of the two groups Sp is a single bond and the other group Sp is different from a single bond.

Preferred compounds of formula I, I-1 to 1-9 and I-1-1 to I-6-11 are selected from compounds wherein
n=m=0, or
n=1 an m=1, or
n=m=1, and/or
n=m=0 and one of ring B and ring D is a single bond, and/or
ring C denotes naphthalene-1,4-diyl or anthracene-9,10-diyl, or
ring C denotes benzene-1,4-diyl which is substituted by ethyl, and/or
P denotes acrylate or methacrylate and/or
Sp denotes Sp″-X″, preferably, —Sp″-X″— denotes —(CH₂)ₚ₁—, —(CH₂)ₚ₁—O—, —(CH₂)ₚ₁—O—CO—, —(CH₂)ₚ₁—CO—O—, —(CH₂)ₚ₁—O—CO—O—, —(CH₂CH₂₀)_{q1}—CH₂CH₂—, —CH₂CH₂—S—CH₂CH₂—, or —CH₂CH₂—NH—CH₂CH₂—, in which p1 is an integer from 1 to 12, q1 is an integer from 1 to 3, and/or
if R¹¹ or R is P-Sp-, both groups P-Sp- are identical, or
if R¹¹ or R is P-Sp-, one of the groups Sp is a single bond and the other of the groups Sp is different from a single bond, and/or
L denotes methyl or ethyl, preferably ethyl, and r denotes 1, and/or
L denotes methyl or ethyl, and r denotes 2, and/or
r denotes 2 and the two substituents L are attached to directly adjacent C atoms and form, together with the C atoms to which they are attached, a cyclic group, preferably a cyclohexenyl group, with 5 or 6 C atoms, and/or
ring C is substituted by one L which denotes P-Sp-, preferably acrylate,
R or R¹¹ is alkyl, alkoxy, thioalkyl, alkylcarbonyl, alkoxycarbonyl, alkylcarbonyloxy or alkoxycarbonyloxy preferably with 1 to 15 C atoms which is more preferably optionally fluorinated, very preferably methyl, methoxy, ethyl, ethoxy, propyl, ispropyl, propyloxy, isopropyloxy, or straight-chain or branched butyl or butyloxy.

Further preferred compounds of formula I are outlined in the example section below.

The synthesis of the compounds of formula I and its subformulae can be carried out analogously to the illustrative reactions shown below or in the examples. The preparation of further compounds according to the invention can also be carried out by other methods known per se to the person skilled in the art from the literature.

Exemplarily, the compounds of formula I can be synthesized according to or in analogy to the methods as illustrated in the Examples.

The compounds of formula I either taken alone or in combination with other RMs in an RM mixture, exhibit in particular and preferably at the same time, a high birefringence, exhibit a good solubility in commonly known organic solvents used in mass production, show an improved broadening potential in chiral RM mixtures, have favorable transition temperatures, and show high resistance against yellowing after being exposed to UV light.

The concentration of the compounds of formula I in the RM mixture is preferably from 35 to 99%, very preferably from 50 to 99%.

Preferably the RM mixture comprises one or more RMs having only one polymerisable functional group (monoreactive RMs), and one or more RMs having two or more polymerisable functional groups (di- or multireactive RMs).

The di- or multireactive RMs are preferably selected of formula DRM

P¹-Sp¹-MG-Sp²-P² DRM

wherein
P1 and P2 independently of each other denote a polymerisable group,
Sp1 and Sp2 independently of each other are a spacer group or a single bond, and
MG is a rod-shaped mesogenic group, which is preferably selected of formula MG

-(A¹-Z¹)ₙ-A² MG

wherein
A¹ and A² denote, in case of multiple occurrence independently of one another, an aromatic or alicyclic group, which optionally contains one or more heteroatoms selected from N, O and S, and is optionally mono- or polysubstituted by L,
L is P-Sp-, F, Cl, Br, I, —CN, —NO₂, —NCO, —NCS, —OCN, —SCN, —C(═O)NR^{x}R^{y}, —C(═O)OR^{x}, —C(═O)R^{x}, —NR^{x}R^{y}, —OH, —SF₅, optionally substituted silyl, aryl or heteroaryl with 1 to 12, preferably 1 to 6 C atoms, and straight chain or branched alkyl, alkoxy, alkylcarbonyl, alkoxycarbonyl, alkylcarbonyloxy or alkoxycarbonyloxy with 1 to 12, preferably 1 to 6 C atoms, wherein one or more H atoms are optionally replaced by F or Cl,
R^{x} and R^{y} independently of each other denote H or alkyl with 1 to 12 C-atoms,
Z¹ denotes, in case of multiple occurrence independently of one another, —O—, —S—, —CO—, —COO—, —OCO—, —S—CO—, —CO—S—, —O—COO—, —CO—NR⁰⁰—, —NR⁰⁰—CO—, —NR⁰⁰—CO—NR⁰⁰⁰, —NR⁰⁰—CO—O—, —O—CO—NR⁰⁰—, —OCH₂—, —CH₂O—, —SCH₂—, —CH₂S—, —CF₂O—, —OCF₂—, —CF₂S—, —SCF₂—, —CH₂CH₂—, —(CH₂)ₙ₁, —CF₂CH₂—, —CH₂CF₂—, —CF₂CF₂—, —CH═N—, —N═CH—, —N═N—, —CH═CR⁰⁰—, —CY₁═CY₂—, —C≡C—, —CH═CH—COO—, —OCO—CH═CH— or a single bond, preferably —COO—, —OCO— or a single bond,
Y¹ and Y² independently of each other denote H, F, Cl or CN,
n is 1, 2, 3 or 4, preferably 1 or 2, most preferably 2,
n1 is an integer from 1 to 10, preferably 1, 2, 3 or 4.

Preferred groups A¹ and A² include, without limitation, furan, pyrrol, thiophene, oxazole, thiazole, thiadiazole, imidazole, phenylene, cyclohexylene, bicyclooctylene, cyclohexenylene, pyridine, pyrimidine, pyrazine, azulene, indane, fluorene, naphthalene, tetrahydronaphthalene, anthracene, phenanthrene and dithienothiophene, all of which are unsubstituted or substituted by 1, 2, 3 or 4 groups L as defined above.

Particular preferred groups A¹ and A² are selected from 1,4-phenylene, pyridine-2,5-diyl, pyrimidine-2,5-diyl, thiophene-2,5-diyl, naphthalene-2,6-diyl, 1,2,3,4-tetrahydro-naphthalene-2,6-diyl, indane-2,5-diyl, bicyclooctylene or 1,4-cyclohexylene wherein one or two non-adjacent CH₂ groups are optionally replaced by O and/or S, wherein these groups are unsubstituted or substituted by 1, 2, 3 or 4 groups L as defined above.

Preferred RMs of formula DRM are selected of formula DRMa

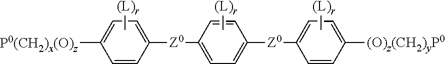

### DRMa

wherein
P⁰ is, in case of multiple occurrence independently of one another, a polymerisable group, preferably an acryl, methacryl, oxetane, epoxy, vinyl, heptadiene, vinyloxy, propenyl ether or styrene group,
Z⁰ is —COO—, —OCO—, —CH₂CH₂—, —CF₂O—, —OCF₂—, —C═C—, —CH═CH—,—OCO—CH═CH—, —CH═CH—COO—, or a single bond,
L has on each occurrence identically or differently one of the meanings given for L¹ in formula I, and is preferably, in case of multiple occurrence independently of one another, selected from F, Cl, CN or optionally halogenated alkyl, alkoxy, alkylcarbonyl, alkoxycarbonyl, alkylcarbonyloxy or alkoxycarbonyloxy with 1 to 5 C atoms,
r is 0, 1, 2, 3 or 4,
x and y are independently of each other 0 or identical or different integers from 1 to 12,
Z is 0 or 1, with z being 0 if the adjacent x or y is 0.

Very preferred RMs of formula DRM are selected from the following formulae:

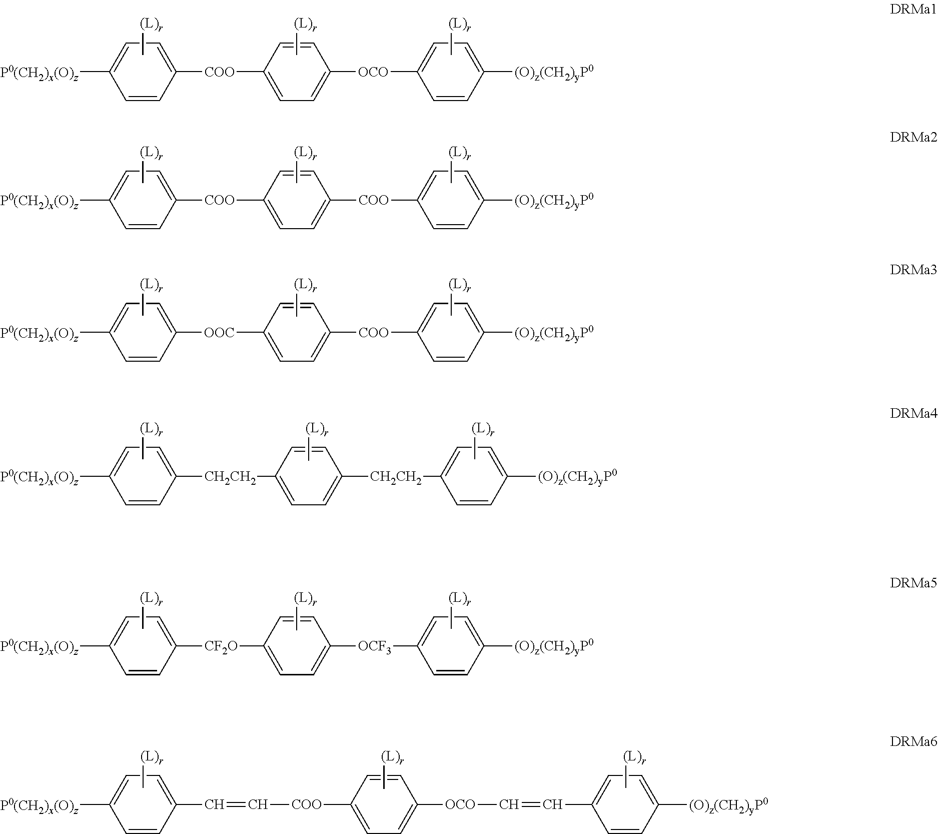

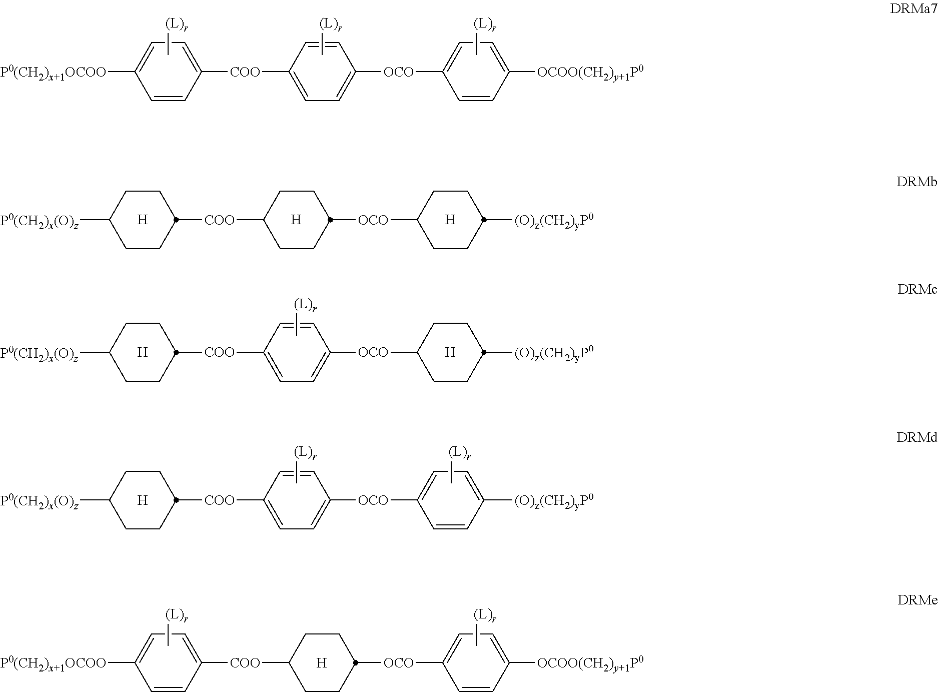

wherein P⁰, L, r, x, y and z are as defined in formula DRMa.

Especially preferred are compounds of formula DRMa1, DRMa2 and DRMa3, in particular those of formula DRMa1.

The concentration of di- or multireactive RMs, preferably those of formula DRM and its subformulae, in the RM mixture is preferably from 1% to 60%, very preferably from 2 to 40%.

In another preferred embodiment the RM mixture comprises, in addition to the compounds of formula I, one or more monoreactive RMs. These additional monoreactive RMs are preferably selected from formula MRM:

P¹-Sp¹-MG-R MRM

wherein P¹, Sp¹ and MG have the meanings given in formula DRM,
R denotes P-Sp-, F, Cl, Br, I, —CN, —NO₂, —NCO, —NCS, —OCN, —SCN, —C(═O)NR^{x}R^{y}, —C(═O)X, —C(═O)OR^{x}, —C(═O)R^{y}, —NR^{x}R^{y}, —OH, —SF₅, optionally substituted silyl, straight chain or branched alkyl, alkoxy, alkylcarbonyl, alkoxycarbonyl, alkylcarbonyloxy or alkoxycarbonyloxy with 1 to 12, preferably 1 to 6 C atoms, wherein one or more H atoms are optionally replaced by F or Cl,
X is halogen, preferably F or Cl, and
R^{x} and R^{y} are independently of each other H or alkyl with 1 to 12 C-atoms.

Preferably the RMs of formula MRM are selected from the following formulae.

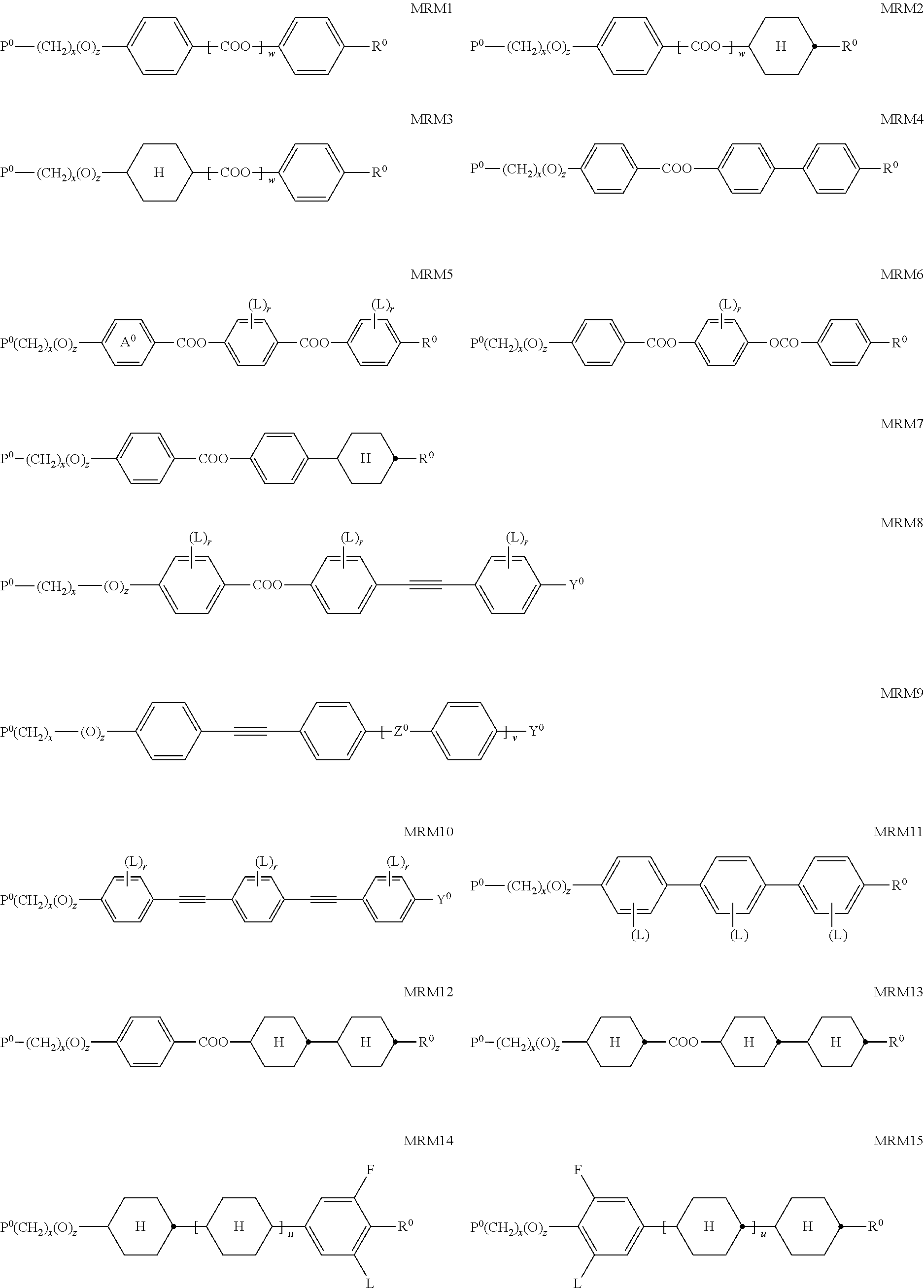

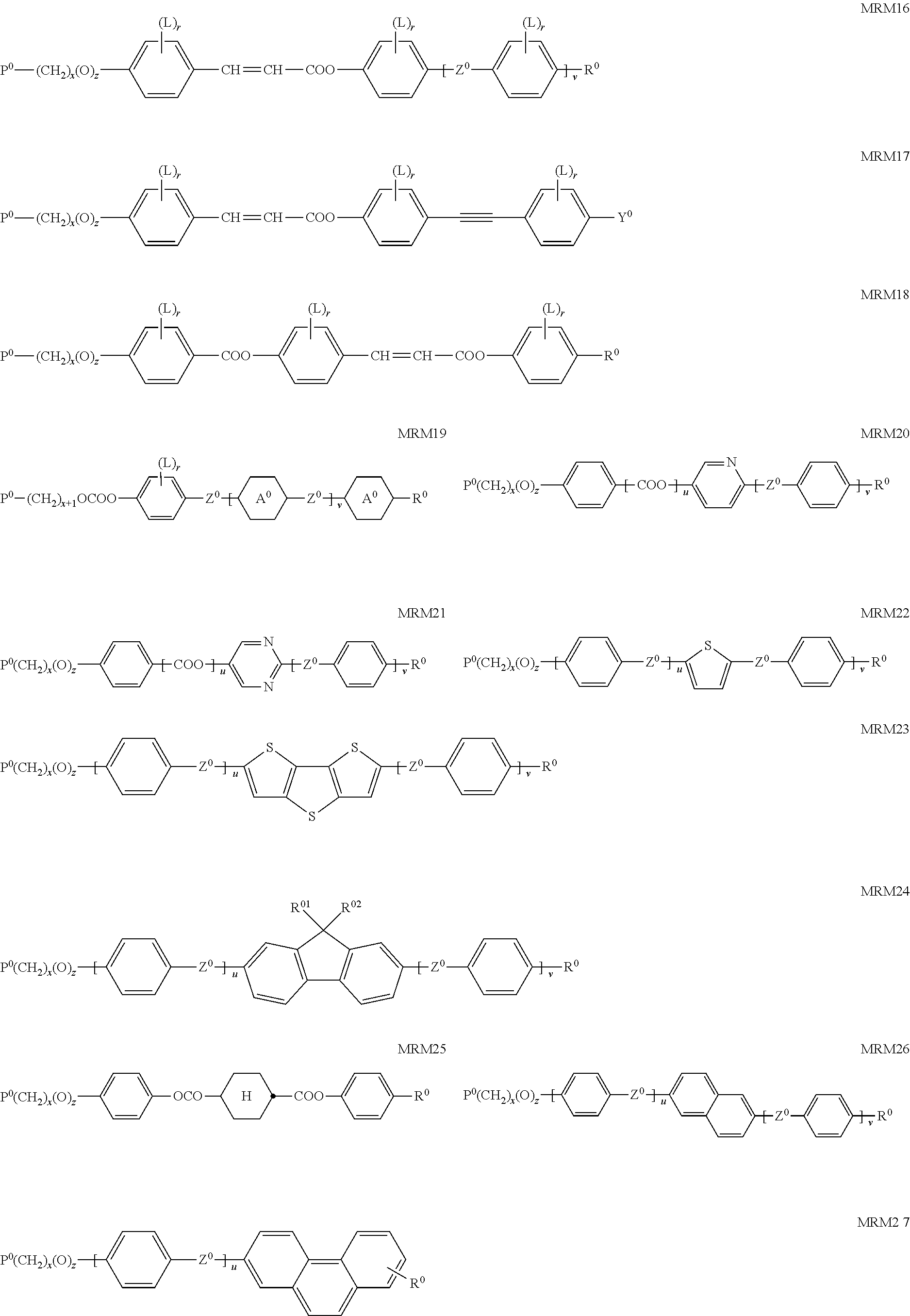

wherein P⁰, L, r, x, y and z are as defined in formula DRMa,
R⁰, R⁰¹
and R⁰² are each an independently alkyl, alkoxy, thioalkyl, alkylcarbonyl, alkoxycarbonyl, alkylcarbonyloxy or alkoxycarbonyloxy with 1 or more, preferably 1 to 15 C atoms or denotes Y⁰ or P—(CH₂)_{y}—(O)_{z}—,
X⁰ is —O—, —S—, —CO—, —COO—, —OCO—, —O—COO—, —CO—NR⁰¹—, —NR⁰¹—CO—, —NR⁰¹—CO—NR⁰¹—, —OCH₂—, —CH₂O—, —SCH₂—, —CH₂S—, —CF₂O—, —OCF₂—, —CF₂S—, —SCF₂—, —CF₂CH₂—, —CH₂CF₂—, —CF₂CF₂—, —CH═N—, —N═CH—, —N═N—, —CH═CR⁰¹−, —CF═CF—, —C≡C—, —CH═CH—COO—, —OCO—CH═CH— or a single bond
Y⁰ is F, Cl, CN, NO₂, OCH₃, OCN, SCN, SF₅, or mono- oligo- or polyfluorinated alkyl or alkoxy with 1 to 4 C atoms,
Z⁰ is —COO—, —OCO—, —CH₂CH₂—, —CF₂O—, —OCF₂—, —CH═CH—,—OCO—CH═CH—, —CH═CH—COO—, or a single bond,
A⁰ is, in case of multiple occurrence independently of one another, 1,4-phenylene that is unsubstituted or substituted with 1, 2, 3 or 4 groups L, or trans-1,4-cyclohexylene,
R^{01,02} are independently of each other H, R⁰ or Y⁰,
u and v are independently of each other 0, 1 or 2,
W is 0 or 1,
and wherein the benzene and naphthalene rings can additionally be substituted with one or more identical or different groups L.

Especially preferred are compounds of formula MRM1, MRM2, MRM3, MRM4, MRM5, MRM6, MRM7, MRM9 and MRM10, in particular those of formula MRM1, MRM4, MRM6, and MRM7.

The concentration of the monoreactive RMs, preferably those of formula MRM, in the RM mixture is preferably from 1 to 80%, very preferably from 5 to 20%.

The RM mixture preferably exhibits a nematic LC phase, or a smectic LC phase and a nematic LC phase, very preferably a nematic LC phase at room temperature.

In formulae DRM, MRM and their preferred subformulae, L is preferably selected from F, Cl, CN, NO₂ or straight chain or branched alkyl, alkoxy, alkylcarbonyl, alkoxycarbonyl, alkylcarbonlyoxy or alkoxycarbonyloxy with 1 to 12 C atoms, wherein the alkyl groups are optionally perfluorinated, or P-Sp-.

Very preferably L is selected from F, Cl, CN, NO₂, CH₃, C₂H₅, C(CH₃)₃, CH(CH₃)₂, CH₂CH(CH₃)C₂H₅, OCH₃, OC₂H₅, COCH₃, COC₂H₅, COOCH₃, COOC₂H₅, CF₃, OCF₃, OCHF₂, OC₂F₅ or P-Sp-, in particular F, Cl, CN, CH₃, C₂H₅, C(CH₃)₃, CH(CH₃)₂, OCH₃, COCH₃ or OCF₃, most preferably F, Cl, CH₃, C(CH₃)₃, OCH₃ or COCH₃, or P-Sp-.

Preferably the RM mixture according to the present invention optionally comprises one or more chiral compounds. These chiral compounds may be non-mesogenic compounds or mesogenic compounds. Additionally, these chiral compounds, whether mesogenic or non-mesogenic, may be non-reactive, monoreactive or multireactive.

Preferably the utilized chiral compounds have each alone or in combination with each other an absolute value of the helical twisting power (IHTPₜₒₜₐₗ) of 20 μm⁻¹ or more, preferably of 40 μm⁻¹ or more, more preferably in the range of 60 μm⁻¹ or more, most preferably in the range of 80 μm⁻¹ or more to 260 μm⁻¹, in particular those disclosed in WO 98/00428.

Preferably, non-polymerisable chiral compounds are selected from the group of compounds of formulae C-I to C-III,

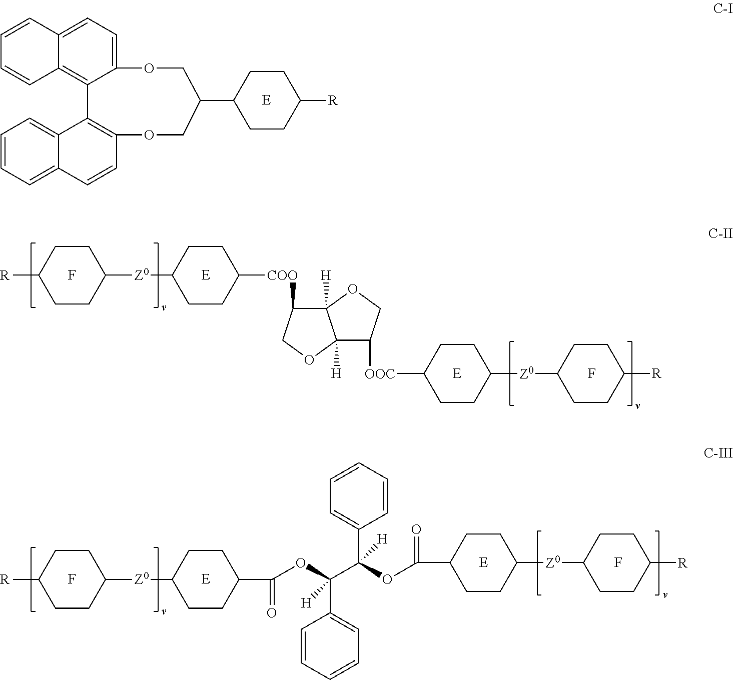

the latter ones including the respective (S,S) enantiomers,
wherein E and F are each independently 1,4-phenylene or trans-1,4-cyclo-hexylene, v is 0 or 1, Z⁰ is —COO—, —OCO—, —CH₂CH₂— or a single bond, and R is alkyl, alkoxy or alkanoyl with 1 to 12 C atoms.

Particularly preferred liquid-crystalline media comprise one or more chiral compounds, which do not necessarily have to show a liquid crystalline phase.

The compounds of formula C-II and their synthesis are described in WO 98/00428. Especially preferred is the compound CD-1, as shown in table D below. The compounds of formula C-III and their synthesis are described in GB 2 328 207.

Further, typically used chiral compounds are e.g. the commercially available R/S-5011, CD-1, R/S-811 and CB-15 (from Merck KGaA, Darmstadt, Germany).

The above mentioned chiral compounds R/S-5011 and CD-1 and the (other) compounds of formulae C-I, C-II and C-III exhibit a very high helical twisting power (HTP), and are therefore particularly useful for the purpose of the present invention.

The RM mixture preferably comprises 1 to 5, in particular 1 to 3, very preferably 1 or 2 chiral compounds, preferably selected from the above formula C-II, in particular CD-1, and/or formula C-III and/or R-5011 or S-5011, very preferably, the chiral compound is R-5011, S-5011 or CD-1.

Preferably the RM mixture optionally comprise one or more non-reactive chiral compound and/or one or more reactive chiral compounds, which are preferably selected from mono- and/or multireactive chiral compounds.

Suitable mesogenic reactive chiral compounds preferably comprise one or more ring elements, linked together by a direct bond or via a linking group and, where two of these ring elements optionally may be linked to each other, either directly or via a linking group, which may be identical to or different from the linking group mentioned. The ring elements are preferably selected from the group of four-, five-, six- or seven-, preferably of five- or six-, membered rings.

Suitable polymerisable chiral compounds and their synthesis are described in U.S. Pat. No. 7,223,450.

Preferred mono-reactive chiral compounds are selected from compounds of formula CRM.

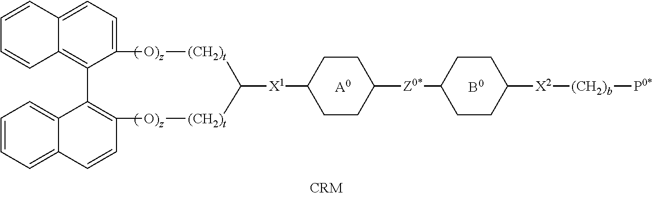

wherein
P⁰* P, with P being a polymerisable group
A⁰ and B⁰ are, in case of multiple occurrence independently of one another, 1,4-phenylene that is unsubstituted or substituted with 1, 2, 3 or 4 groups L as defined above, or trans-1,4-cyclohexylene,
X¹ and X² are independently of each other —O—, —COO—, —OCO—, —O—CO—O— or a single bond,
Z⁰* is, in case of multiple occurrence independently of one another, —COO—, —OCO—, —O—CO—O—, —OCH₂—, —CH₂O—, —CF₂O—, —OCF₂—, —CH₂CH₂—, —(CH₂)₄—, —CF₂CH₂—, —CH₂CF₂—, —CF₂CF₂—, —C≡C—, —CH═CH—, —CH═CH—COO—, —OCO—CH═CH— or a single bond,
t is, independently of each other 0, 1, 2 or 3,
a is 0, 1 or 2,
b is 0 or an integer from 1 to 12,
Z is 0 or 1,
and wherein the naphthalene rings can additionally be substituted with one or more identical or different groups L
wherein
L is, independently of each other F, Cl, CN, halogenated alkyl, alkoxy, alkylcarbonyl, alkoxycarbonyl, alkylcarbonyloxy or alkoxycarbonyloxy with 1 to 5 C atoms.

The compounds of formula CRM are preferably selected from the group of compounds of formulae CRM-a.

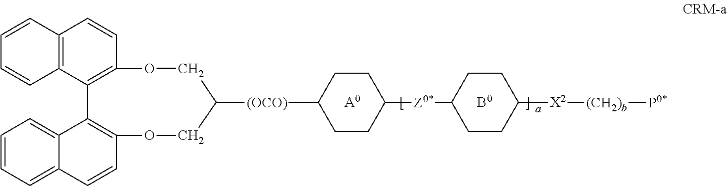

wherein A⁰, B⁰, Z⁰*, P⁰*, a and b have the meanings given in formula CRM or one of the preferred meanings given above and below, and (OCO) denotes —O—CO— or a single bond.

Especially preferred compounds of formula CRM are selected from the group consisting of the following subformulae:

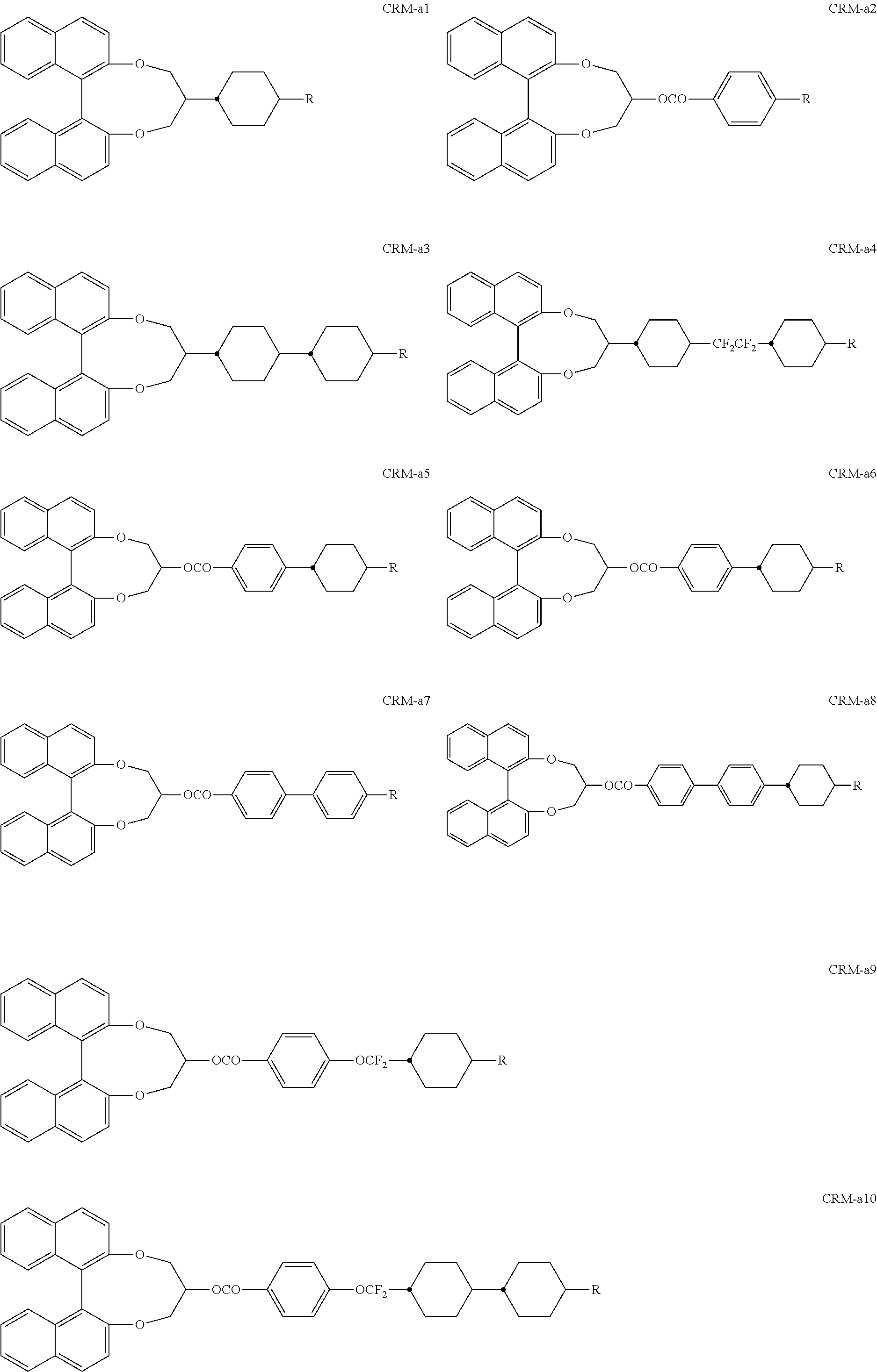

wherein R is —X²—(CH₂)ₓ—P⁰* as defined in formula CRM-a, and the benzene and naphthalene rings are unsubstituted or substituted with 1, 2, 3 or 4 groups L as defined above and below.

The amount of chiral compounds in the liquid-crystalline medium is preferably from 1 to 20%, more preferably from 1 to 15%, even more preferably 1 to 10%, and most preferably 2 to 6%, by weight of the total mixture.

Another object of the invention is an RM formulation comprising one or more compounds of formula I, or comprising an RM mixture, as described above and below, and further comprising one or more solvents and/or additives.

In a preferred embodiment the RM formulation comprises optionally one or more additives selected from the group consisting of polymerisation initiators, surfactants, stabilisers, catalysts, sensitizers, inhibitors, chain-transfer agents, co-reacting monomers, reactive thinners, surface-active compounds, lubricating agents, wetting agents, dispersing agents, hydrophobing agents, adhesive agents, flow improvers, degassing or defoaming agents, deaerators, diluents, reactive diluents, auxiliaries, colourants, dyes, pigments and nanoparticles.

In another preferred embodiment the RM formulation optionally comprises one or more additives selected from polymerisable non-mesogenic compounds (reactive thinners). The amount of these additives in the RM formulation is preferably from 0 to 30%, very preferably from 0 to 25%.

The reactive thinners used are not only substances which are referred to in the actual sense as reactive thinners, but also auxiliary compounds already mentioned above which contain one or more complementary reactive units, for example hydroxyl, thiol-, or amino groups, via which a reaction with the polymerizable units of the liquid-crystalline compounds can take place.

The substances which are usually capable of photopolymerization include, for example, mono-, bi- and polyfunctional compounds containing at least one olefinic double bond. Examples thereof are vinyl esters of carboxylic acids, for example of lauric, myristic, palmitic and stearic acid, and of dicarboxylic acids, for example of succinic acid, adipic acid, allyl and vinyl ethers and methacrylic and acrylic esters of monofunctional alcohols, for example of lauryl, myristyl, palmityl and stearyl alcohol, and diallyl and divinyl ethers of bifunctional alcohols, for example ethylene glycol and 1,4-butanediol.

Also suitable are, for example, methacrylic and acrylic esters of polyfunctional alcohols, in particular those which contain no further functional groups, or at most ether groups, besides the hydroxyl groups. Examples of such alcohols are bifunctional alcohols, such as ethylene glycol, propylene glycol and their more highly condensed representatives, for example diethylene glycol, triethylene glycol, dipropylene glycol, tripropylene glycol etc., butanediol, pentanediol, hexanediol, neopentyl glycol, alkoxylated phenolic compounds, such as ethoxylated and propoxylated bisphenols, cyclohexanedimethanol, trifunctional and polyfunctional alcohols, such as glycerol, trimethylolpropane, butanetriol, trimethylolethane, pentaerythritol, ditrimethylolpropane, dipentaerythritol, sorbitol, mannitol, and the corresponding alkoxylated, in particular ethoxylated and propoxylated alcohols.

Other suitable reactive thinners are polyester (meth)acrylates, which are the (meth)acrylic ester of polyesterols.

Examples of suitable polyesterols are those which can be prepared by esterification of polycarboxylic acids, preferably dicarboxylic acids, using polyols, preferably diols. The starting materials for such hydroxyl-containing polyesters are known to the person skilled in the art. Dicarboxylic acids which can be employed are succinic, glutaric acid, adipic acid, sebacic acid, o-phthalic acid and isomers and hydrogenation products thereof, and esterifiable and transesterifiable derivatives of said acids, for example anhydrides and dialkyl esters. Suitable polyols are the abovementioned alcohols, preferably ethyleneglycol, 1,2- and 1,3-propylene glycol, 1,4-butanediol, 1,6-hexanediol, neopentyl glycol, cyclohexanedimethanol and polyglycols of the ethylene glycol and propylene glycol type.

Suitable reactive thinners are furthermore 1,4-divinylbenzene, triallyl cyanurate, acrylic esters of tricyclodecenyl alcohol of the following formula

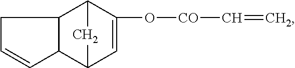 [SMILES: C=CC(=O)OC1=CC2CC1C1CC=CC21]

also known under the name dihydrodicyclopentadienyl acrylate, and the allyl esters of acrylic acid, methacrylic acid and cyanoacrylic acid.

Of the reactive thinners which are mentioned by way of example, those containing photopolymerizable groups are used in particular and in view of the abovementioned preferred compositions.

This group includes, for example, dihydric and polyhydric alcohols, for example ethylene glycol, propylene glycol and more highly condensed representatives thereof, for example diethylene glycol, triethylene glycol, dipropylene glycol, tripropylene glycol etc., butanediol, pentanediol, hexanediol, neopentyl glycol, cyclohexanedimethanol, glycerol, trimethylolpropane, butanetriol, trimethylolethane, pentaerythritol, ditrimethylolpropane, dipentaerythritol, sorbitol, mannitol and the corresponding alkoxylated, in particular ethoxylated and propoxylated alcohols.

The group furthermore also includes, for example, alkoxylated phenolic compounds, for example ethoxylated and propoxylated bisphenols.

These reactive thinners may furthermore be, for example, epoxide or urethane (meth)acrylates.

Epoxide (meth)acrylates are, for example, those as obtainable by the reaction, known to the person skilled in the art, of epoxidized olefins or poly- or diglycidyl ether, such as bisphenol A diglycidyl ether, with (meth)acrylic acid.

Urethane (meth)acrylates are, in particular, the products of a reaction, likewise known to the person skilled in the art, of hydroxylalkyl (meth)acrylates with poly- or diisocyanates.

Such epoxide and urethane (meth)acrylates are included amongst the compounds listed above as “mixed forms”.

If reactive thinners are used, their amount and properties must be matched to the respective conditions in such a way that, on the one hand, a satisfactory desired effect, for example the desired colour of the composition according to the invention, is achieved, but, on the other hand, the phase behaviour of the liquid-crystalline composition is not excessively impaired. The low-crosslinking (high-crosslinking) liquid-crystalline compositions can be prepared, for example, using corresponding reactive thinners which have a relatively low (high) number of reactive units per molecule.

The group of diluents include, for example:
C1-C₄-alcohols, for example methanol, ethanol, n-propanol, isopropanol, butanol, isobutanol, sec-butanol and, in particular, the C5-C12-alcohols n-pentanol, n-hexanol, n-heptanol, n-octanol, n-nonanol, n-decanol, n-undecanol and n-dodecanol, and isomers thereof, glycols, for example 1,2-ethylene glycol, 1,2- and 1,3-propylene glycol, 1,2-, 2,3- and 1,4-butylene glycol, di- and triethylene glycol and di- and tripropylene glycol, ethers, for example methyl tert-butyl ether, 1,2-ethylene glycol mono- and dimethyl ether, 1,2-ethylene glycol mono- and -diethylether, 3-methoxypropanol, 3-isopropoxypropanol, tetrahydrofuran and dioxane, ketones, for example acetone, methyl ethyl ketone, methyl isobutyl ketone and diacetone alcohol (4-hydroxy-4-methyl-2-pentanone), C1-C₅-alkyl esters, for example methyl acetate, ethyl acetate, propyl acetate, butyl acetate and amyl acetate, aliphatic and aromatic hydrocarbons, for example pentane, hexane, heptane, octane, isooctane, petroleum ether, toluene, xylene, ethylbenzene, tetralin, decalin, dimethylnaphthalene, white spirit, Shellsol® and Solvesso® mineral oils, for example gasoline, kerosine, diesel oil and heating oil, but also natural oils, for example olive oil, soya oil, rapeseed oil, linseed oil and sunflower oil.

It is of course also possible to use mixtures of these diluents in the compositions according to the invention.

So long as there is at least partial miscibility, these diluents can also be mixed with water. Examples of suitable diluents here are C1-C₄-alcohols, for example methanol, ethanol, n-propanol, isopropanol, butanol, isobutanol and sec-butanol, glycols, for example 1,2-ethylene glycol, 1,2- and 1,3-propylene glycol, 1,2-, 2,3- and 1,4-butylene glycol, di- and triethylene glycol, and di- and tripropylene glycol, ethers, for example tetrahydrofuran and dioxane, ketones, for example acetone, methyl ethyl ketone and diacetone alcohol (4-hydroxy-4-methyl-2-pentanone), and C1-C4-alkyl esters, for example methyl, ethyl, propyl and butyl acetate.

The diluents are optionally employed in a proportion of from about 0 to 10.0% by weight, preferably from about 0 to 5.0% by weight, based on the total weight of the RM formulation.

The antifoams and deaerators (c1)), lubricants and flow auxiliaries (c2)), thermally curing or radiation-curing auxiliaries (c3)), substrate wetting auxiliaries (c4)), wetting and dispersion auxiliaries (c5)), hydrophobicizing agents (c6)), adhesion promoters (c7)) and auxiliaries for promoting scratch resistance (c8)) cannot strictly be delimited from one another in their action.

For example, lubricants and flow auxiliaries often also act as antifoams and/or deaerators and/or as auxiliaries for improving scratch resistance. Radiation-curing auxiliaries can also act as lubricants and flow auxiliaries and/or deaerators and/or as substrate wetting auxiliaries. In individual cases, some of these auxiliaries can also fulfil the function of an adhesion promoter (c8)).

Corresponding to the above-said, a certain additive can therefore be classified in a number of the groups c1) to c8) described below.

The antifoams in group c1) include silicon-free and silicon-containing polymers. The silicon-containing polymers are, for example, unmodified or modified polydialkylsiloxanes or branched copolymers, comb or block copolymers comprising polydialkylsiloxane and polyether units, the latter being obtainable from ethylene oxide or propylene oxide.

The deaerators in group c1) include, for example, organic polymers, for example polyethers and polyacrylates, dialkylpolysiloxanes, in particular dimethylpolysiloxanes, organically modified polysiloxanes, for example arylalkyl-modified polysiloxanes, and fluorosilicones.

The action of the antifoams is essentially based on preventing foam formation or destroying foam that has already formed. Antifoams essentially work by promoting coalescence of finely divided gas or air bubbles to give larger bubbles in the medium to be deaerated, for example the compositions according to the invention, and thus accelerate escape of the gas (of the air). Since antifoams can frequently also be employed as deaerators and vice versa, these additives have been included together under group c1).

Such auxiliaries are, for example, commercially available from Tego as TEGO® Foamex 800, TEGO® Foamex 805, TEGO® Foamex 810, TEGO® Foamex 815, TEGO® Foamex 825, TEGO® Foamex 835, TEGO® Foamex 840, TEGO® Foamex 842, TEGO® Foamex 1435, TEGO® Foamex 1488, TEGO® Foamex 1495, TEGO® Foamex 3062, TEGO® Foamex 7447, TEGO® Foamex 8020, Tego® Foamex N, TEGO® Foamex K 3, TEGO® Antifoam 2-18, TEGO® Antifoam 2-18, TEGO® Antifoam 2-57, TEGO® Antifoam 2-80, TEGO® Antifoam 2-82, TEGO® Antifoam 2-89, TEGO® Antifoam 2-92, TEGO® Antifoam 14, TEGO® Antifoam 28, TEGO® Antifoam 81, TEGO® Antifoam D 90, TEGO® Antifoam 93, TEGO® Antifoam 200, TEGO® Antifoam 201, TEGO® Antifoam 202, TEGO® Antifoam 793, TEGO® Antifoam 1488, TEGO® Antifoam 3062, TEGOPREN® 5803, TEGOPREN® 5852, TEGOPREN® 5863, TEGOPREN® 7008, TEGO® Antifoam 1-60, TEGO® Antifoam 1-62, TEGO® Antifoam 1-85, TEGO® Antifoam 2-67, TEGO® Antifoam WM 20, TEGO® Antifoam 50, TEGO® Antifoam 105, TEGO® Antifoam 730, TEGO® Antifoam MR 1015, TEGO® Antifoam MR 1016, TEGOR Antifoam 1435, TEGO® Antifoam N, TEGO® Antifoam KS 6, TEGO® Antifoam KS 10, TEGO® Antifoam KS 53, TEGO® Antifoam KS 95, TEGO® Antifoam KS 100, TEGO® Antifoam KE 600, TEGO® Antifoam KS 911, TEGO® Antifoam MR 1000, TEGO® Antifoam KS 1100, Tego® Airex 900, Tego® Airex 910, Tego® Airex 931, Tego® Airex 935, Tego® Airex 936, Tego® Airex 960, Tego® Airex 970, Tego® Airex 980 and Tego® Airex 985 and from BYK as BYK®-011, BYK®-019, BYK®-020, BYK®-021, BYK®-022, BYK®-023, BYK®-024, BYK®-025, BYK®-027, BYK®-031, BYK®-032, BYK®-033, BYK®-034, BYK®-035, BYK®-036, BYK®-037, BYK®-045, BYK®-051, BYK®-052, BYK®-053, BYK®-055, BYK®-057, BYK®-065, BYK®-066, BYK®-070, BYK®-080, BYK®-088, BYK®-141 and BYK®-A 530.

The auxiliaries in group c1) are optionally employed in a proportion of from about 0 to 3.0% by weight, preferably from about 0 to 2.0% by weight, based on the total weight of the RM formulation.

In group c2), the lubricants and flow auxiliaries typically include silicon-free, but also silicon-containing polymers, for example polyacrylates or modifiers, low-molecular-weight polydialkylsiloxanes. The modification consists in some of the alkyl groups having been replaced by a wide variety of organic radicals. These organic radicals are, for example, polyethers, polyesters or even long-chain alkyl radicals, the former being used the most frequently.

The polyether radicals in the correspondingly modified polysiloxanes are usually built up from ethylene oxide and/or propylene oxide units. Generally, the higher the proportion of these alkylene oxide units in the modified polysiloxane, the more hydrophilic is the resultant product.

Such auxiliaries are, for example, commercially available from Tego as TEGO® Glide 100, TEGO® Glide ZG 400, TEGO® Glide 406, TEGO® Glide 410, TEGO® Glide 411, TEGO® Glide 415, TEGO® Glide 420, TEGO® Glide 435, TEGO® Glide 440, TEGO® Glide 450, TEGO® Glide A 115, TEGO® Glide B 1484 (can also be used as antifoam and deaerator), TEGO® Flow ATF, TEGO® Flow 300, TEGO® Flow 460, TEGO® Flow 425 and TEGO® Flow ZFS 460. Suitable radiation-curable lubricants and flow auxiliaries, which can also be used to improve the scratch resistance, are the products TEGO® Rad 2100, TEGO® Rad 2200, TEGO® Rad 2500, TEGO® Rad 2600 and TEGO® Rad 2700, which are likewise obtainable from TEGO.

Such-auxiliaries are available, for example, from BYK as BYK®-300 BYK®-306, BYK®-307, BYK®-310, BYK®-320, BYK®-333, BYK®-341, Byk® 354, Byk®361, Byk®361N, BYK®388.

The auxiliaries in group c2) are optionally employed in a proportion of from about 0 to 3.0% by weight, preferably from about 0 to 2.0% by weight, based on the total weight of the RM formulation.

In group c3), the radiation-curing auxiliaries include, in particular, polysiloxanes having terminal double bonds which are, for example, a constituent of an acrylate group. Such auxiliaries can be crosslinked by actinic or, for example, electron radiation. These auxiliaries generally combine a number of properties together. In the uncrosslinked state, they can act as antifoams, deaerators, lubricants and flow auxiliaries and/or substrate wetting auxiliaries, while, in the crosslinked state, they increase, in particular, the scratch resistance, for example of coatings or films which can be produced using the compositions according to the invention. The improvement in the gloss properties, for example of precisely those coatings or films, is regarded essentially as a consequence of the action of these auxiliaries as antifoams, deaerators and/or lubricants and flow auxiliaries (in the uncrosslinked state).

Examples of suitable radiation-curing auxiliaries are the products TEGO® Rad 2100, TEGO® Rad 2200, TEGO® Rad 2500, TEGO® Rad 2600 and TEGO® Rad 2700 available from TEGO and the product BYK®-371 available from BYK.

Thermally curing auxiliaries in group c3) contain, for example, primary OH groups which are able to react with isocyanate groups, for example of the binder.

Examples of thermally curing auxiliaries which can be used are the products BYK®-370, BYK®-373 and BYK®-375 available from BYK.

The auxiliaries in group c3) are optionally employed in a proportion of from about 0 to 5.0% by weight, preferably from about 0 to 3.0% by weight, based on the total weight of the RM formulation.

The substrate wetting auxiliaries in group c4) serve, in particular, to increase the wettability of the substrate to be printed or coated, for example, by printing inks or coating compositions, for example compositions according to the invention. The generally attendant improvement in the lubricant and flow behaviour of such printing inks or coating compositions has an effect on the appearance of the finished (for example crosslinked) print or coating.

A wide variety of such auxiliaries are commercially available, for example from Tego as TEGO® Wet KL 245, TEGO® Wet 250, TEGO® Wet 260 and TEGO® Wet ZFS 453 and from BYK as BYK®-306, BYK®-307, BYK®-310, BYK®-333, BYK®-344, BYK®-345, BYK®-346 and Byk®-348.

The auxiliaries in group c4) are optionally employed in a proportion of from about 0 to 3.0% by weight, preferably from about 0 to 1.5% by weight, based on the total weight of the liquid-crystalline composition.

The wetting and dispersion auxiliaries in group c5) serve, in particular, to prevent the flooding and floating and the sedimentation of pigments and are therefore, if necessary, suitable in particular in pigmented compositions according to the invention.

These auxiliaries stabilize pigment dispersions essentially through electrostatic repulsion and/or steric hindrance of the pigment particles containing these additives, where, in the latter case, the interaction of the auxiliary with the ambient medium (for example binder) plays a major role.

Since the use of such wetting and dispersion auxiliaries is common practice, for example in the technical area of printing inks and paints, the selection of a suitable auxiliary of this type generally does not present the person skilled in the art with any difficulties, if they are used.

Such wetting and dispersion auxiliaries are commercially available, for example from Tego, as TEGO® Dispers 610, TEGO® Dispers 610 S, TEGO® Dispers 630, TEGO® Dispers 700, TEGO® Dispers 705, TEGO® Dispers 710, TEGO® Dispers 720 W, TEGO® Dispers 725 W, TEGO® Dispers 730 W, TEGO® Dispers 735 W and TEGO® Dispers 740 W and from BYK as Disperbyk®, Disperbyk®-107, Disperbyk®-108, Disperbyk®-110, Disperbyk®-111, Disperbyk®-115, Disperbyk®-130, Disperbyk®-160, Disperbyk®-161, Disperbyk®-162, Disperbyk®-163, Disperbyk®-164, Disperbyk®-165, Disperbyk®-166, Disperbyk®-167, Disperbyk®-170, Disperbyk®-174, Disperbyk®-180, Disperbyk®-181, Disperbyk®-182, Disperbyk®-183, Disperbyk®-184, Disperbyk®-185, Disperbyk®-190, Anti-Terra®-U, Anti-Terra®-U 80, Anti-Terra®-P, Anti-Terra®-203, Anti-Terra®-204, Anti-Terra®-206, BYK®-151, BYK®-154, BYK®-155, BYK®-P 104 S, BYKR-P 105, Lactimon®, Lactimon®-WS and Bykumen®.

The amount of the auxiliaries in group c5) used on the mean molecular weight of the auxiliary. In any case, a preliminary experiment is therefore advisable, but this can be accomplished simply by the person skilled in the art.

The hydrophobicizing agents in group c6) can be used to give water-repellent properties to prints or coatings produced, for example, using compositions according to the invention. This prevents or at least greatly suppresses swelling due to water absorption and thus a change in, for example, the optical properties of such prints or coatings. In addition, when the composition is used, for example, as a printing ink in offset printing, water absorption can thereby be prevented or at least greatly reduced.

Such hydrophobicizing agents are commercially available, for example, from Tego as Tego® Phobe WF, Tego® Phobe 1000, Tego® Phobe 1000 S, Tego® Phobe 1010, Tego® Phobe 1030, Tego® Phobe 1010, Tego® Phobe 1010, Tego® Phobe 1030, Tego® Phobe 1040, Tego® Phobe 1050, Tego® Phobe 1200, Tego® Phobe 1300, Tego® Phobe 1310 and Tego® Phobe 1400.

The auxiliaries in group c6) are optionally employed in a proportion of from about 0 to 5.0% by weight, preferably from about 0 to 3.0% by weight, based on the total weight of the RM formulation.

Adhesion promoters from group c7) serve to improve the adhesion of two interfaces in contact. It is directly evident from this that essentially the only fraction of the adhesion promoter that is effective is that located at one or the other or at both interfaces. If, for example, it is desired to apply liquid or pasty printing inks, coating compositions or paints to a solid substrate, this generally means that the adhesion promoter must be added directly to the latter or the substrate must be pre-treated with the adhesion promoters (also known as priming), i.e. this substrate is given modified chemical and/or physical surface properties.

If the substrate has previously been primed with a primer, this means that the interfaces in contact are that of the primer on the one hand and of the printing ink or coating composition or paint on the other hand. In this case, not only the adhesion properties between the substrate and the primer, but also between the substrate and the printing ink or coating composition or paint play a part in adhesion of the overall multilayer structure on the substrate.

Adhesion promoters in the broader sense which may be mentioned are also the substrate wetting auxiliaries already listed under group c4), but these generally do not have the same adhesion promotion capacity.

In view of the widely varying physical and chemical natures of substrates and of printing inks, coating compositions and paints intended, for example, for their printing or coating, the multiplicity of adhesion promoter systems is not surprising.

Adhesion promoters based on silanes are, for example, 3-aminopropyltrimethoxysilane, 3-aminopropyltriethoxysilane, 3-aminopropylmethyldiethoxysilane, N-aminoethyl-3-aminopropyltrimethoxysilane, N-aminoethyl-3-aminopropylmethyldimethoxysilane, N-methyl-3-aminopropyltrimethoxysilane, 3-ureidopropyltriethoxysilane, 3-methacryloyloxypropyltrimethoxysilane, 3-glycidyloxypropyltrimethoxysilane, 3-mercaptopropyltrimethoxysilane, 3-chloropropyltrimethoxysilane and vinyltrimethoxysilane. These and other silanes are commercially available from Hüls, for example under the tradename DYNASILAN®.

Corresponding technical information from the manufacturers of such additives should generally be used or the person skilled in the art can obtain this information in a simple manner through corresponding preliminary experiments.

However, if these additives are to be added as auxiliaries from group c7) to the RM formulations according to the invention, their proportion optionally corresponds to from about 0 to 5.0% by weight, based on the total weight of the RM formulation. These concentration data serve merely as guidance, since the amount and identity of the additive are determined in each individual case by the nature of the substrate and of the printing/coating composition. Corresponding technical information is usually available from the manufacturers of such additives for this case or can be determined in a simple manner by the person skilled in the art through corresponding preliminary experiments.

The auxiliaries for improving the scratch resistance in group c8) include, for example, the abovementioned products TEGO® Rad 2100, TEGO® Rad 2200, TEGO® Rad 2500, TEGO® Rad 2600 and TEGO® Rad 2700, which are available from Tego.

For these auxiliaries, the amount data given for group c3) are likewise suitable, i.e. these additives are optionally employed in a proportion of from about 0 to 5.0% by weight, preferably from about 0 to 3.0% by weight, based on the total weight of the liquid-crystalline composition.

Examples which may be mentioned of light, heat and/or oxidation stabilizers are the following:
alkylated monophenols, such as 2,6-di-tert-butyl-4-methylphenol, 2-tert-butyl-4,6-dimethylphenol, 2,6-di-tert-butyl-4-ethylphenol, 2,6-di-tert-butyl-4-n-butylphenol, 2,6-di-tert-butyl-4-isobutylphenol, 2,6-dicyclopentyl-4-methylphenol, 2-(a-methylcyclohexyl)-4,6-dimethylphenol, 2,6-dioctadecyl-4-methylphenol, 2,4,6-tricyclohexylphenol, 2,6-di-tert-butyl-4-methoxymethylphenol, nonylphenols which have a linear or branched side chain, for example 2,6-dinonyl-4-methylphenol, 2,4-dimethyl-6-(1′-methylundec-1′-yl)phenol, 2,4-dimethyl-6-(1′-methylheptadec-1′-yl)phenol, 2,4-dimethyl-6-(1′-methyltridec-1′-yl)phenol and mixtures of these compounds, alkylthiomethylphenols, such as 2,4-dioctylthiomethyl-6-tert-butylphenol, 2,4-dioctylthiomethyl-6-methylphenol, 2,4-dioctylthiomethyl-6-ethylphenol and 2,6-didodecylthiomethyl-4-nonylphenol,
Hydroquinones and alkylated hydroquinones, such as 2,6-di-tert-butyl-4-methoxyphenol, 2,5-di-tert-butylhydroquinone, 2,5-di-tert-amylhydrocrainone, 2,6-diphenyl-4-octadecyloxyphenol, 2,6-di-tert-butylhydroquinone, 2,5-di-tert-butyl-4-hydroxyanisole, 3,5-di-tert-butyl-4-hydroxyanisole, 3,5-di-tert-butyl-4-hydroxyphenyl stearate and bis(3,5-di-tert-butyl-4-hydroxyphenyl)adipate,
Tocopherols, such as α-tocopherol, β-tocopherol, γ-tocopherol, δ-tocopherol and mixtures of these compounds, and tocopherol derivatives, such as tocopheryl acetate, succinate, nicotinate and polyoxyethylenesuccinate (“tocofersolate”),
hydroxylated diphenyl thioethers, such as 2,2′-thiobis(6-tert-butyl-4-methylphenol), 2,2′-thiobis(4-octylphenol), 4,4′-thiobis(6-tert-butyl-3-methylphenol), 4,4′-thiobis(6-tert-butyl-2-methylphenol), 4,4′-thiobis(3,6-di-sec-amylphenol) and 4,4′-bis(2,6-dimethyl-4-hydroxyphenyl)disulfide,
Alkylidenebisphenols, such as 2,2′-methylenebis(6-tert-butyl-4-methylphenol), 2,2′-methylenebis(6-tert-butyl-4-ethylphenol), 2,2′-methylenebis[4-methyl-6-(a-methylcyclohexyl)phenol], 2,2′-methylenebis(4-methyl-6-cyclohexylphenol), 2,2′-methylenebis(6-nonyl-4-methylphenol), 2,2′-methylenebis(4,6-di-tert-butylphenol), 2,2-ethylidenebis(4,6-di-tert-butylphenol), 2,2′-ethylidenebis(6-tert-butyl-4-isobutylphenol), 2,2′-methylenebis[6-(a-methylbenzyl)-4-nonylphenol], 2,2′-methylenebis[6-(a,a-dimethylbenzyl)-4-nonylphenol], 4,4′-methylenebis(2,6-di-tert-butylphenol), 4,4′-methylenebis(6-tert-butyl-2-methylphenol), 1,1-bis(5-tert-butyl-4-hydroxy-2-methylphenyl)butane, 2,6-bis(3-tert-butyl-5-methyl-2-hydroxybenzyl)-4-methylphenol, 1,1,3-tris(5-tert-butyl-4-hydroxy-2-methylphenyl)butane, 1, 1-bis(5-tert-butyl-4-hydroxy-2-methylphenyl)-3-n-dodecyl-mercaptobutane, ethylene glycol bis[3,3-bis(3′-tert-butyl-4′-hydroxyphenyl)butyrate], bis(3-tert-butyl-4-hydroxy-5-methylphenyl)dicyclopentadiene, bis[2-(3′-tert-butyl-2′-hydroxy-5′-methylbenzyl)-6-tert-butyl-4-methylphenyl]terephthalate, 1,1-bis(3,5-dimethyl-2-hydroxyphenyl)butane, 2,2-bis(3,5-di-tert-butyl-4-hydroxyphenyl)propane, 2,2-bis(5-tert-butyl-4-hydroxy-2-methylphenyl)-4-n-dodecyl-mercaptobutane and 1,1,5,5-tetrakis(5-tert-butyl-4-hydroxy-2-methylphenyl)pentane,
O-, N- and S-benzyl compounds, such as 3,5,3′,5′-tetra-tert-butyl-4,4′-dihydroxydibenzyl ether, octadecyl 4-hydroxy-3,5-dimethylbenzylmercaptoacetate, tridecyl 4-hydroxy-3,5-di-tert-butylbenzylmercaptoacetate, tris(3,5-di-tert-butyl-4-hydroxybenzyl)amine, bis(4-tert-butyl-3-hydroxy-2,6-dimethylbenzyl)dithioterephthalate, bis(3,5-di-tert-butyl-4-hydroxybenzyl)sulfide and isooctyl-3,5-di-tert-butyl-4-hydroxybenzylmercaptoacetate,
aromatic hydroxybenzyl compounds, such as 1,3,5-tris(3,5-di-tert-butyl-4-hydroxybenzyl)-2,4,6-trimethyl-benzene, 1,4-bis(3,5-di-tert-butyl-4-hydroxybenzyl)-2,3,5,6-tetramethyl-benzene and 2,4,6-tris(3,5-di-tert-butyl-4-hydroxybenzyl)phenol,
Triazine compounds, such as 2,4-bis(octylmercapto)-6-(3,5-di-tert-butyl-4-hydroxyanilino)-1,3,5-triazine, 2-octylmercapto-4,6-bis(3,5-di-tert-butyl-4-hydroxyanilino)-1,3,5-triazine, 2-octylmercapto-4,6-bis(3,5-di-tert-butyl-4-hydroxyphenoxy)-1,3,5-triazine, 2,4,6-tris(3,5-di-tert-butyl-4-hydroxyphenoxy)-1,2,3-triazine, 1,3,5-tris(3,5-di-tert-butyl-4-hydroxybenzyl)isocyanurate, 1,3,5-tris(4-tert-butyl-3-hydroxy-2,6-dimethylbenzyl)isocyanurate, 2,4,6-tris(3,5-di-tert-butyl-4-hydroxyphenylethyl)-1,3,5-triazine, 1,3,5-tris-(3,5-di-tert-butyl-4-hydroxyphenylpropionyl)hexahydro-1,3,5-triazine, 1,3,5-tris(3,5-dicyclohexyl-4-hydroxybenzyl)isocyanurate and 1,3,5-tris(2-hydroxyethyl)isocyanurate,
Benzylphosphonates, such as dimethyl 2,5-di-tert-butyl-4-hydroxybenzylphosphonate, diethyl 3,5-di-tert-butyl-4-hydroxybenzylphosphonate, dioctadecyl 3,5-di-tert-butyl-4-hydroxybenzylphosphonate and dioctadecyl 5-tert-butyl-4-hydroxy-3-methylbenzylphosphonate,
Acylaminophenols, such as 4-hydroxylauroylanilide, 4-hydroxystearoylanilide and octyl N-(3,5-di-tert-butyl-4-hydroxyphenyl)carbamate,
Propionic and acetic esters, for example of monohydric or polyhydric alcohols, such as methanol, ethanol, n-octanol, i-octanol, octadecanol, 1,6-hexanediol, 1,9-nonanediol, ethylene glycol, 1,2-propanediol, neopentyl glycol, thiodiethylene glycol, diethylene glycol, triethylene glycol, pentaerythritol, tris(hydroxyethyl)isocyanurate, N,N′-bis(hydroxyethyl)oxalamide, 3-thiaundecanol, 3-thiapentadecanol, trimethylhexanediol, trimethylolpropane and 4-hydroxymethyl-1-phospha-2,6,7-trioxabicyclo[2.2.2]-octane,
Propionamides based on amine derivatives, such as N, N′-bis(3,5-di-tert-butyl-4-hydroxyphenylpropionyl)hexamethylenediamine, N, N′-bis(3,5-di-tert-butyl-4-hydroxyphenylpropionyl)trimethylenediamine and N,N′-bis(3,5-di-tert-butyl-4-hydroxyphenylpropionyl)hydrazine,
Ascorbic acid (Vitamin C) and ascorbic acid derivatives, such as ascorbyl palmitate, laurate and stearate, and ascorbyl sulfate and phosphate,
Antioxidants based on amine compounds, such as N, N′-diisopropyl-p-phenylenediamine, N,N′-di-sec-butyl-p-phenylenediamine, N, N′-bis(1,4-dimethylpentyl)-p-phenylenediamine, N, N′-bis(1-ethyl-3-methylpentyl)-p-phenylenediamine, N,N′-bis(1-methylheptyl)-p-phenylenediamine, N,N′-dicyclohexyl-p-phenylenediamine, N,N′-diphenyl-p-phenylenediamine, N, N′-bis(2-naphthyl)-p-phenylenediamine, N-isopropyl-N′-phenyl-p-phenylenediamine, N-(1,3-dimethylbutyl)-N′-phenyl-p-phenylenediamine, N-(1-methylheptyl)-N′-phenyl-p-phenylenediamine, N-cyclohexyl-N′-phenyl-p-phenylenediamine, 4-(p-toluenesulfamoyl)diphenylamine, N, N′-dimethyl-N,N′-di-sec-butyl-p-phenylenediamine, diphenylamine, N-allyldiphenylamine, 4-isopropoxydiphenylamine, N-phenyl-1-naphthylamine, N-(4-tert-octylphenyl)-1-naphthylamine, N-phenyl-2-naphthylamine, octyl-substituted diphenylamine, such as p,p′-di-tert-octyldiphenylamine, 4-n-butylaminophenol, 4-butyrylaminophenol, 4-nonanoylaminophenol, 4-dodecanoylaminophenol, 4-octadecanoylaminophenol, bis[4-methoxyphenyl)amine, 2,6-di-tert-butyl-4-dimethylaminomethylphenol, 2,4-diaminodiphenylmethane, 4,4′-diaminodiphenylmethane, N, N,N′, N′-tetramethyl-4,4′-diaminodiphenylmethane, 1,2-bis[(2-methylphenyl)amino]ethane, 1,2-bis(phenylamino)propane, (o-tolyl)biguanide, bis[4-(1′,3′-dimethylbutyl)phenyl]amine, tert-octyl-substituted N-phenyl-1-naphthylamine, a mixture of mono- and dialkylated tert-butyl/tert-octyldiphenylamine, a mixture of mono- and dialkylated nonyldiphenylamine, a mixture of mono- and dialkylated dodecyldiphenylamine, a mixture of mono- and dialkylated isopropyl/isohexyldiphenylamine, a mixture of mono- and dialkylated tert-butyldiphenylamine, 2,3-dihydro-3,3-dimethyl-4H-1,4-benzothiazine, phenothiazine, a mixture of mono- and dialkylated tert-butyl/tert-octylphenothiazine, a mixture of mono- and dialkylated tert-octylphenothiazine, N-allylphenothiazine, N, N, N′, N′-tetraphenyl-1,4-diaminobut-2-ene, N, N-bis(2,2,6,6-tetramethylpiperidin-4-yl)hexamethylenediamine, bis(2,2,6,6-tetramethylpiperidin-4-yl)sebacate, 2,2,6,6-tetramethylpiperidin-4-one and 2,2,6,6-tetramethylpiperidin-4-ol,
Phosphines, Phosphites and phosphonites, such as triphenylphosnine triphenylphosphite, diphenyl alkyl phosphite, phenyl dialkyl phosphite, tris(nonylphenyl)phosphite, trilauryl phosphite, trioctadecyl phosphite, distearyl pentaerythritol diphosphite, tris(2,4-di-tert-butylphenyl)phosphite, diisodecyl pentaerythritol diphosphite, bis(2,4-di-tert-butylphenyl)pentaerythritol diphosphite, bis(2,6-di-tert-butyl-4-methylphenyl)pentaerythritol diphosphite, diisodecyloxy pentaerythritol diphosphite, bis(2,4-di-tert-butyl-6-methylphenyl)pentaerythritol diphosphite, bis(2,4,6-tris(tert-butylphenyl))pentaerythritol diphosphite, tristearyl sorbitol triphosphite, tetrakis(2,4-di-tert-butylphenyl)4,4′-biphenylenediphosphonite, 6-isooctyloxy-2,4,8, 10-tetra-tert-butyl-12H-dibenz[d,g]-1,3,2-dioxaphosphocine, 6-fluoro-2,4,8, 10-tetra-tert-butyl-12-methyl-dibenz[d,g]-1,3,2-dioxaphosphocine, bis(2,4-di-tert-butyl-6-methylphenyl)methyl phosphite and bis(2,4-di-tert-butyl-6-methylphenyl)ethyl phosphite,
2-(2′-Hydroxyphenyl)benzotriazoles, such as 2-(2′-hydroxy-5′-methylphenyl)benzotriazole, 2-(3′,5′-di-tert-butyl-2′-hydroxyphenyl)benzotriazole, 2-(5′-tert-butyl-2′-hydroxyphenyl)benzotriazole, 2-(2′-hydroxy-5′-(1,1,3,3-tetramethylbutyl)phenyl)benzotriazole, 2-(3′,5′-di-tert-butyl-2′-hydroxyphenyl)-5-chlorobenzotriazole, 2-(3′-tert-butyl-2′-hydroxy-5′-methylphenyl)-5-chlorobenzotriazole, 2-(3′-sec-butyl-5′-tert-butyl-2′-hydroxyphenyl)benzotriazole, 2-(2′-hydroxy-4′-octyloxyphenyl)benzotriazole, 2-(3′,5′-di-tert-amyl-2′-hydroxyphenyl)benzotriazole, 2-(3,5′-bis-(α,α-dimethylbenzyl)-2′-hydroxyphenyl)benzotriazole, a mixture of 2-(3′-tert-butyl-2′-hydroxy-5′-(2-octyloxycarbonylethyl)phenyl)-5-chlorobenzotriazole, 2-(3′-tert-butyl-5′-[2-(2-ethylhexyloxy)carbonylethyl]-2′-hydroxy phenyl)-5-chlorobenzotriazole, 2-(3′-tert-butyl-2′-hydroxy-5′-(2-methoxycarbonylethyl)phenyl)-5-chlorobenzotriazole, 2-(3′-tert-butyl-2′-hydroxy-5′-(2-methoxycarbonylethyl)phenyl)benzotriazole, 2-(3′-tert-butyl-2′-hydroxy-5′-(2-octyloxycarbonylethyl)phenyl)benzotriazole, 2-(3′-tert-butyl-5′-[2-(2-ethylhexyloxy)carbonylethyl]-2′-hydroxy phenyl)benzotriazole, 2-(3′-dodecyl-2′-hydroxy-5′-methylphenyl)benzotriazole and 2-(3′-tert-butyl-2′-hydroxy-5′-(2-isooctyloxycarbonylethyl)phenyl benzotriazole, 2,2′-methylenebis[4-(1, 1,3,3-tetramethylbutyl)-6-benzotriazol-2-ylphenol]; the product of complete esterification of 2-[3′-tert-butyl-5′-(2-methoxycarbonylethyl)-2′-hydroxyphenyl]-2H-benzotriazole with polyethylene glycol 300;
sulfur-containing peroxide scavengers and sulfur-containing antioxidants, such as esters of 3,3′-thiodipropionic acid, for example the lauryl, stearyl, myristyl and tridecyl esters, mercaptobenzimidazole and the zinc salt of 2-mercaptobenzimidazole, dibutylzinc dithiocarbamates, dioctadecyl disulfide and pentaerythritol tetrakis(B-dodecylmercapto)propionate, 2-hydroxybenzophenones, such as the 4-hydroxy, 4-methoxy, 4-octyloxy, 4-decycloxy, 4-dodecyloxy, 4-benzyloxy, 4,2′,4′-trihydroxy and 2′-hydroxy-4,4′-dimethoxy derivatives,
Esters of unsubstituted and substituted benzoic acids, such as 4-tert-butylphenyl salicylate, phenyl salicylate, octylphenyl salicylate, dibenzoylresorcinol, bis(4-tert-butylbenzoyl)resorcinol, benzoylresorcinol, 2,4-di-tert-butylphenyl 3,5-di-tert-butyl-4-hydroxybenzoate, hexadecyl-3,5-di-tert-butyl-4-hydroxybenzoate, octadecyl-3,5-di-tert-butyl-4-hydroxybenzoate and 2-methyl-4,6-di-tert-butylphenyl-3,5-di-tert-butyl-4-hydroxybenzoate,
Acrylates, such as ethyl a-cyano-ß,ß-diphenylacrylate, isooctyl α-cyano-β,β-diphenylacrylate, methyl α-methoxycarbonylcinnamate, methyl α-cyano-β-methyl-p-methoxycinnamate, butyl-α-cyano-β-methyl-p-methoxycinnamate and methyl-α-methoxycarbonyl-p-methoxycinnamate, sterically hindered amines, such as bis(2,2,6,6-tetramethylpiperidin-4-yl)sebacate, bis(2,2,6,6-tetramethylpiperidin-4-yl)succinate, bis(1,2,2,6,6-pentamethylpiperidin-4-yl)sebacate, bis(1-octyloxy-2,2,6,6-tetramethylpiperidin-4-yl)sebacate, bis(1,2,2,6,6-pentamethylpiperidin-4-yl)-n-butyl-3,5-di-tert-butyl-4-hydroxybenzylmalonate, the condensation product of 1-(2-hydroxyethyl)-2,2,6,6-tetramethyl-4-hydroxypiperidine and succinic acid, the condensation product of N,N′-bis(2,2,6,6-tetramethylpiperidin-4-yl)hexamethylenediamine and 4-tert-octylamino-2,6-dichloro-1,3,5-triazine, tris(2,2,6,6-tetramethylpiperidin-4-yl)nitrilotriacetate, tetrakis(2,2,6,6-tetramethylpiperidin-4-yl)1,2,3,4-butanetetracarboxylate, 1,1′-(1,2-ethylene)bis(3,3,5,5-tetramethylpiperazinone), 4-benzoyl-2,2,6,6-tetramethylpiperidine, 4-stearyloxy-2,2,6,6-tetramethylpiperidine, bis(1,2,2,6,6-pentamethylpiperidin-4-yl)2-n-butyl-2-(2-hydroxy-3,5-di-tert-butylbenzyl)malonate, 3-n-octyl-7,7,9,9-tetramethyl-1,3,8-triazaspiro[4.5]decane-2,4-dione, bis(1-octyloxy-2,2,6,6-tetramethylpiperidin-4-yl)sebacate, bis(1-octyloxy-2,2,6,6-tetramethylpiperidin-4-yl)succinate, the condensation product of N,N′-bis(2,2,6,6-tetramethylpiperidin-4-yl)hexamethylenediamine and 4-morpholino-2,6-dichloro-1,3,5-triazine, the condensation product of 2-chloro-4,6-bis(4-n-butylamino-2,2,6,6-tetramethylpiperidin-4-yl)-1,3,5-triazine and 1,2-bis(3-aminopropylamino)ethane, the condensation product of 2-chloro-4,6-di(4-n-butylamino-1,2,2,6,6-pentamethylpiperidin-4-yl)-1,3,5-triazine and 1,2-bis(3-aminopropylamino)ethane, 8-acetyl-3-dodecyl-7,7,9,9-tetramethyl-1,3,8-triazaspiro[4.5]-decane-2,4-dione, 3-dodecyl-1-(2,2,6,6-tetramethylpiperidin-4-yl)pyrrolidine-2,5-dione, 3-dodecyl-1-(1,2,2,6,6-pentamethylpiperidin-4-yl)pyrrolidine-2,5-dione, a mixture of 4-hexadecyloxy- and 4-stearyloxy-2,2,6,6-tetramethylpiperidine, the condensation product of N,N′-bis(2,2,6,6-tetramethylpiperidin-4-yl)hexamethylenediamine and 4-cyclohexylamino-2,6-dichloro-1,3,5-triazine, the condensation product of 1,2-bis(3-aminopropylamino)ethane and 2,4,6-trichloro-1,3,5-triazine, 4-butylamino-2,2,6,6-tetramethylpiperidine, N-(2,2,6,6-tetramethylpiperidin-4-yl)-n-dodecylsuccinimide, N-(1,2,2,6,6-pentamethylpiperidin-4-yl)-n-dodecylsuccinimide, 2-undecyl-7,7,9,9-tetramethyl-1-oxa-3,8-diaza-4-oxo-spiro[4.5]-decane, the condensation product of 7,7,9,9-tetramethyl-2-cycloundecyl-1-oxa-3,8-diaza-4-oxospiro-[4.5]decane and epichlorohydrin, the condensation products of 4-amino-2,2,6,6-tetramethylpiperidine with tetramethylolacetylenediureas and poly(methoxypropyl-3-oxy)-[4(2,2,6,6-tetramethyl)piperidinyl]-siloxane,
Oxalamides, such as 4,4′-dioctyloxyoxanilide, 2,2′-diethoxyoxanilide, 2,2′-dioctyloxy-5,5′-di-tert-butoxanilide, 2,2′-didodecyloxy-5,5′-di-tert-butoxanilide, 2-ethoxy-2′-ethyloxanilide, N, N′-bis(3-dimethylaminopropyl)oxalamide, 2-ethoxy-5-tert-butyl-2′-ethoxanilide and its mixture with 2-ethoxy-2′-ethyl-5,4′-di-tert-butoxanilide, and mixtures of ortho-, para-methoxy-disubstituted oxanilides and mixtures of ortho- and para-ethoxy-disubstituted oxanilides, and
2-(2-hydroxyphenyl)-1,3,5-triazines, such as 2,4,6-tris-(2-hydroxy-4-octyloxyphenyl)-1,3,5-triazine, 2-(2-hydroxy-4-octyloxyphenyl)-4,6-bis(2,4-dimethylphenyl)-1,3,5-triazine, 2-(2,4-dihydroxyphenyl)-4,6-bis(2,4-dimethylphenyl)-1,3,5-triazine, 2,4-bis(2-hydroxy-4-propyloxyphenyl)-6-(2,4-dimethylphenyl)-1,3,5-triazine, 2-(2-hydroxy-4-octyloxyphenyl)-4,6-bis(4-methylphenyl)-1,3,5-triazine, 2-(2-hydroxy-4-dodecyloxyphenyl)-4,6-bis(2,4-dimethylphenyl)-1,3,5-triazine, 2-(2-hydroxy-4-tridecyloxyphenyl)-4,6-bis(2,4-dimethylphenyl)-1,3,5-triazine, 2-[2-hydroxy-4-(2-hydroxy-3-butyloxypropoxy)phenyl]-4,6-bis(2,4-dimethyl)-1,3,5-triazine, 2-[2-hydroxy-4-(2-hydroxy-3-octyloxypropoxy)phenyl]-4,6-bis(2,4-dimethyl)-1,3,5-triazine, 2-[4-(dodecyloxy/tridecyloxy-2-hydroxypropoxy)-2-hydroxyphenyl]-4,6-bis(2,4-dimethylphenyl)-1,3,5-triazine, 2-[2-hydroxy-4-(2-hydroxy-3-dodecyloxypropoxy)phenyl]-4,6-bis-(2,4-dimethylphenyl)-1,3,5-triazine, 2-(2-hydroxy-4-hexyloxyphenyl)-4,6-diphenyl-1,3,5-triazine, 2-(2-hydroxy-4-methoxyphenyl)-4,6-diphenyl-1,3,5-triazine, 2,4,6-tris[2-hydroxy-4-(3-butoxy-2-hydroxypropoxy)phenyl]-1,3,5-triazine and 2-(2-hydroxyphenyl)-4-(4-methoxyphenyl)-6-phenyl-1,3,5-triazine.

In another preferred embodiment the RM formulation comprises one or more specific antioxidant additives, preferably selected from the Irganox® series, e.g. the commercially available antioxidants Irganox®1076 and Irganox®1010, from Ciba, Switzerland.

In another preferred embodiment, the RM formulation comprises a combination of one or more, more preferably of two or more photoinitiators, for example, selected from the commercially available Irgacure® or Darocure® (Ciba AG) series, in particular, Irgacure 127, Irgacure 184, Irgacure 369, Irgacure 651, Irgacure 817, Irgacure 907, Irgacure 1300, Irgacure, Irgacure 2022, Irgacure 2100, Irgacure 2959, or Darcure TPO, further selected from the commercially available OXE02 (Ciba AG), NCI 930, N1919T (Adeka), SPI-03 or SPI-04 (Samyang).

The concentration of the polymerization initiator(s) as a whole in the RM formulation is preferably from 0.5 to 10%, very preferably from 0.8 to 8%, more preferably 1 to 6%.

In a preferred embodiment the RM formulation is dissolved in a suitable solvent, which are preferably selected from organic solvents.

The solvents are preferably selected from ketones such as acetone, methyl ethyl ketone, methyl propyl ketone, methyl isobutyl ketone or cyclohexanone; acetates such as methyl, ethyl or butyl acetate or methyl acetoacetate; alcohols such as methanol, ethanol or isopropyl alcohol; aromatic solvents such as toluene or xylene; alicyclic hydrocarbons such as cyclopentane or cyclohexane; halogenated hydrocarbons such as di- or trichloromethane; glycols or their esters such as PGMEA (propyl glycol monomethyl ether acetate), γ-butyrolactone. It is also possible to use binary, ternary or higher mixtures of the above solvents. In particular, for multilayer applications, methyl iso butyl ketone is the preferred utilized solvent

In case the RM formulation contains one or more solvents, the total concentration of all solids, including the RMs, in the solvent(s) is preferably from 10 to 60%, more preferably from 20 to 50%, in particular from 30 to 45%

Preferably, the RM formulation comprises besides one or more compounds or formula I,
a) optionally one or more multi— or direactive polymerizable mesogenic compounds, preferably selected from compounds of formula DRM and corresponding subformulae, and/or
b) optionally one or more chiral mesogenic compounds, preferably selected from compounds of formula CRM and its subformulae, and/or
c) optionally one or more monoreactive mesogens, preferably selected from compounds of formula MRM and corresponding subformulae, and/or
d) optionally one or more photoinitiators, and/or
e) optionally one or more antioxidative additives, and/or
f) optionally one or more adhesion promotors, and/or
g) optionally one or more surfactants, and/or
h) optionally one or more mono-, di- or multireactive polymerizable non-mesogenic compounds, and/or
i) optionally one or more dyes showing an absorption maximum at the wavelength used to initiate photo polymerization, and/or
j) optionally one or more chain transfer agents, and/or
k) optionally one or more (UV) stabilizers, and/or
l) optionally one or more lubricants and flow auxiliaries, and
m) optionally one or more diluents, and/or
n) optionally a non-polymerizable nematic component, and/or
o) optionally one or more organic solvents.

More preferably, the RM formulation comprises,
a) one or more compounds of formula I, or its corresponding preferred subformulae,
b) optionally one or more, preferably two or more, direactive polymerizable mesogenic compounds, preferably selected from the compounds of formula DRMa-1,
c) optionally one or more, preferably two or more, monoreactive polymerizable mesogenic compounds, preferably selected from compounds of formulae MRM-1, and/or MRM-4, and/or MRM-6, and/or MRM-7,
d) optionally one or more chiral mesogenic compounds of formula CRM,
e) optionally one or more antioxidative additives, preferably selected from esters of unsubstituted and substituted benzoic acids, in particular Irganox® 1076, and if present, preferably in an amount of 0.01 to 2% by weight, very preferably 0.05 to 1% by weight,
f) optionally one or more photoinitiators, preferably Irgacure®907, and/or SPI-3
g) optionally one or more organic solvents, preferably methyl isobutyl ketone.

The invention further relates to a method of preparing a polymer film by
providing a layer of a RM formulation as described above and below onto a substrate,
polymerizing the polymerizable components of the RM formulation by photopolymerization, and
optionally removing the polymerized film from the substrate and/or optionally providing it onto another substrate.

This RM formulation can be coated or printed onto the substrate, for example by spin-coating, printing, or other known techniques, and the solvent is evaporated off before polymerization. In most cases, it is suitable to heat the mixture in order to facilitate the evaporation of the solvent.

The RM formulation can be applied onto a substrate by conventional coating techniques like spin coating, bar coating or blade coating. It can also be applied to the substrate by conventional printing techniques which are known to the expert, like for example screen printing, offset printing, reel-to-reel printing, letter press printing, gravure printing, rotogravure printing, flexographic printing, intaglio printing, pad printing, heat-seal printing, ink-jet printing or printing by means of a stamp or printing plate.

Suitable substrate materials and substrates are known to the expert and described in the literature, as for example conventional substrates used in the optical films industry, such as glass or plastic. Especially suitable and preferred substrates for polymerization are polyester such as polyethyleneterephthalate (PET) or polyethylenenaphthalate (PEN), polyvinylalcohol (PVA), polycarbonate (PC) triacetylcellulose (TAC), or cyclo olefin polymers (COP), or commonly known color filter materials, in particular triacetylcellulose (TAC), cyclo olefin polymers (COP), or commonly known colour filter materials. Also an optical film obtainable from another or the same RM material can serve as a substrate. This is especially preferred if multilayer systems should be designed comprising one, two, three, four, five or more optical films in an optical component.

The RM formulation preferably exhibits a uniform alignment throughout the whole layer. Preferably the RM formulation exhibits a uniform planar, a uniform homeotropic, uniform cholesteric or patterned alignment.

The Friedel-Creagh-Kmetz rule can be used to predict whether a mixture will adopt planar or homeotropic alignment, by comparing the surface energies of the RM layer (γ_{RM}) and the substrate (γₛ):

If γ_{RM}>γₛ the reactive mesogenic compounds will display homeotropic alignment, If γ_{RM}<γₛ the reactive mesogenic compounds will display homogeneous alignment.

Without to be bound by theory, when the surface energy of a substrate is relatively low, the intermolecular forces between the reactive mesogens are stronger than the forces across the RM-substrate interface and consequently, reactive mesogens align perpendicular to the substrate (homeotropic alignment) in order to maximise the intermolecular forces.

Homeotropic alignment can also be achieved by using amphiphilic materials; they can be added directly to the polymerizable LC material, or the substrate can be treated with these materials in the form of a homeotropic alignment layer. The polar head of the amphiphilic material chemically bonds to the substrate, and the hydrocarbon tail points perpendicular to the substrate. Intermolecular interactions between the amphiphilic material and the RMs promote homeotropic alignment. Commonly used amphiphilic surfactants are described above.

Another method used to promote homeotropic alignment is to apply corona discharge treatment to plastic substrates, generating alcohol or ketone functional groups on the substrate surface. These polar groups can interact with the polar groups present in RMs or surfactants to promote homeotropic alignment.

When the surface tension of the substrate is greater than the surface tension of the RMs, the force across the interface dominates. The interface energy is minimised if the reactive mesogens align parallel with the substrate, so the long axis of the RM can interact with the substrate. One way planar alignment can be promoted is by coating the substrate with a polyimide layer, and then rubbing the alignment layer with a velvet cloth.

Other suitable planar alignment layers are known in the art, like for example rubbed polyimide or alignment layers prepared by photoalignment as described in U.S. Pat. Nos. 5,602,661, 5,389,698 or U.S. Pat. No. 6,717,644.

In general, reviews of alignment techniques are given for example by I. Sage in “Thermotropic Liquid Crystals”, edited by G. W. Gray, John Wiley & Sons, 1987, pages 75-77; and by T. Uchida and H. Seki in “Liquid Crystals—Applications and Uses Vol. 3”, edited by B. Bahadur, World Scientific Publishing, Singapore 1992, pages 1-63. A further review of alignment materials and techniques is given by J. Cognard, Mol. Cryst. Liq. Cryst. 78, Supplement 1 (1981), pages 1-77.

However, it is likewise preferred, that the orientation of the RM molecules vary though the layer thickness. Such as splayed alignments, tilted or twisted alignment types commonly known by the expert.

For the production of the polymer films according to the invention, the polymerizable compounds in the RM formulation are polymerized or crosslinked (if one compound contains two or more polymerizable groups) by in-situ photopolymerization.

The photopolymerization can be carried out in one step. It is also possible to photopolymerize or crosslink the compounds in a second step, which have not reacted in the first step (“end curing”).

In a preferred method of preparation the RM formulation is coated onto a substrate and subsequently photopolymerized for example by exposure to actinic radiation as described for example in WO 01/20394, GB 2,315,072 or WO 98/04651.

Photopolymerization of the LC material is preferably achieved by exposing it to actinic radiation. Actinic radiation means irradiation with light, like UV light, IR light or visible light, irradiation with X-rays or gamma rays, or irradiation with high-energy particles, such as ions or electrons. Preferably, polymerization is carried out by photo irradiation, in particular with UV light. As a source for actinic radiation, for example a single UV lamp or a set of UV lamps can be used. When using a high lamp power the curing time can be reduced. Another possible source for photo radiation is a laser, like e.g. a UV laser, an IR laser, or a visible laser. Another possible source for photo radiation is a LED lamp.

The curing time is dependent, inter alia, on the reactivity of the polymerizable LC material, the thickness of the coated layer, the type of polymerization initiator and the power of the UV lamp. The curing time is preferably ≤5 minutes, very preferably ≤3 minutes, most preferably ≤1 minute. For mass production, short curing times of ≤30 seconds are preferred.

A suitable UV radiation power is preferably in the range from 5 to 200 mWcm−2, more preferably in the range from 50 to 175 mWcm⁻² and most preferably in the range from 100 to 150 mWcm⁻².

In connection with the applied UV radiation and as a function of time, a suitable UV dose is preferably in the range from 25 to 7200 mJcm⁻² more preferably in the range from 100 to 7200 mJcm⁻² and most preferably in the range from 200 to 7200 mJcm⁻².

Photopolymerization is preferably performed under an inert gas atmosphere, preferably in a heated nitrogen atmosphere, but also polymerization in air is possible.

Photopolymerization is preferably performed at a temperature from 1 to 70° C., more preferably 5 to 50° C., even more preferably 15 to 30° C.

The polymerized LC film according to the present invention has good adhesion to plastic substrates, in particular to TAC, COP, and colour filters. Accordingly, it can be used as adhesive or base coating for subsequent LC layers which otherwise would not well adhere to the substrates.

For optical applications of the polymer film, it preferably has a thickness of from 0.5 to 10 μm, very preferably from 0.5 to 5 μm, in particular from 0.5 to 3 μm.

The optical retardation (δ(λ)) of a polymer film as a function of the wavelength of the incident beam (λ) is given by the following equation (7):

δ(λ)=(2πΔn·d)/λ (7)
wherein (Δn) is the birefringence of the film, (d) is the thickness of the film and λ is the wavelength of the incident beam.

According to Snellius law, the birefringence as a function of the direction of the incident beam is defined as

Δn=sinΘ/sinΨ (8)
wherein sinΘ is the incidence angle or the tilt angle of the optical axis in the film and sinΨ is the corresponding reflection angle.

Based on these laws, the birefringence and accordingly optical retardation depends on the thickness of a film and the tilt angle of optical axis in the film (cf. Berek's compensator). Therefore, the skilled expert is aware that different optical retardations or different birefringence can be induced by adjusting the orientation of the liquid-crystalline molecules in the polymer film.

The birefringence (Δn) of the polymer film according to the present invention is preferably in the range from 0.1 to 0.8, more preferable in the range from 0.2 to 0.7 and even more preferable in the range from 0.2 to 0.6.

The optical retardation as a function of the thickness of the polymer film according to the present invention is less than 200 nm, preferable less than 180 nm and even more preferable less than 150 nm.

The polymer film of the present invention can also be used as alignment film or substrate for other liquid-crystalline or RM materials. The inventors have found that the polymer film obtainable from a RM formulation as described above and below, is in particular useful for multilayer applications due to its improved dewetting characteristics. In this way, stacks of optical films or preferably polymerized LC films can be prepared.

In summary, the polymerized LC films and polymerizable LC materials according to the present invention are useful in optical elements like polarisers, compensators, alignment layer, circular polarisers or colour filters in liquid crystal displays or projection systems, decorative images, for the preparation of liquid crystal or effect pigments, and especially in reflective films with spatially varying reflection colours, e.g. as multicolour image for decorative, information storage or security uses, such as non-forgeable documents like identity or credit cards, banknotes etc.

The polymerized LC films according to the present invention can be used in displays of the transmissive or reflective type. They can be used in conventional OLED displays or LCDs, in particular LCDs.

The present invention is described above and below with particular reference to the preferred embodiments. It should be understood that various changes and modifications might be made therein without departing from the spirit and scope of the invention.

Many of the compounds or mixtures thereof mentioned above and below are commercially available. All of these compounds are either known or can be prepared by methods which are known per se, as described in the literature (for example in the standard works such as Houben-Weyl, Methoden der Organischen Chemie [Methods of Organic Chemistry], Georg-Thieme-Verlag, Stuttgart), to be precise under reaction conditions which are known and suitable for said reactions. Use may also be made here of variants which are known per se, but are not mentioned here.

It will be appreciated that variations to the foregoing embodiments of the invention can be made while still falling within the scope of the invention. Alternative features serving the same, equivalent, or similar purpose may replace each feature disclosed in this specification, unless stated otherwise. Thus, unless stated otherwise, each feature disclosed is one example only of a generic series of equivalent or similar features.

All of the features disclosed in this specification may be combined in any combination, except combinations where at least some of such features and/or steps are mutually exclusive. In particular, the preferred features of the invention are applicable to all aspects of the invention and may be used in any combination. Likewise, features described in non-essential combinations may be used separately (not in combination).

It will be appreciated that many of the features described above, particularly of the preferred embodiments, are inventive in their own right and not just as part of an embodiment of the present invention. Independent protection may be sought for these features in addition to or alternative to any invention presently claimed.

The invention will now be described in more detail by reference to the following working examples, which are illustrative only and do not limit the scope of the invention.

Unless explicitly noted otherwise, all temperature values indicated in the present application, such as, for example, for the melting point T(K,N), the transition from the smectic (S) to the nematic (N) phase T(S,N) and the clearing point T(N,I), are quoted in degrees Celsius (° C.). Furthermore, K denots the crystalline state, N denotes the nematic phase, SmX denotes an unidentified smectic phase, X denotes an unidentified mesophase and I denotes the isotropic phase. The data between these symbols represent the transition temperatures in ° C.

### EXAMPLES

Compound (RM-1) is prepared in accordance with the following scheme:

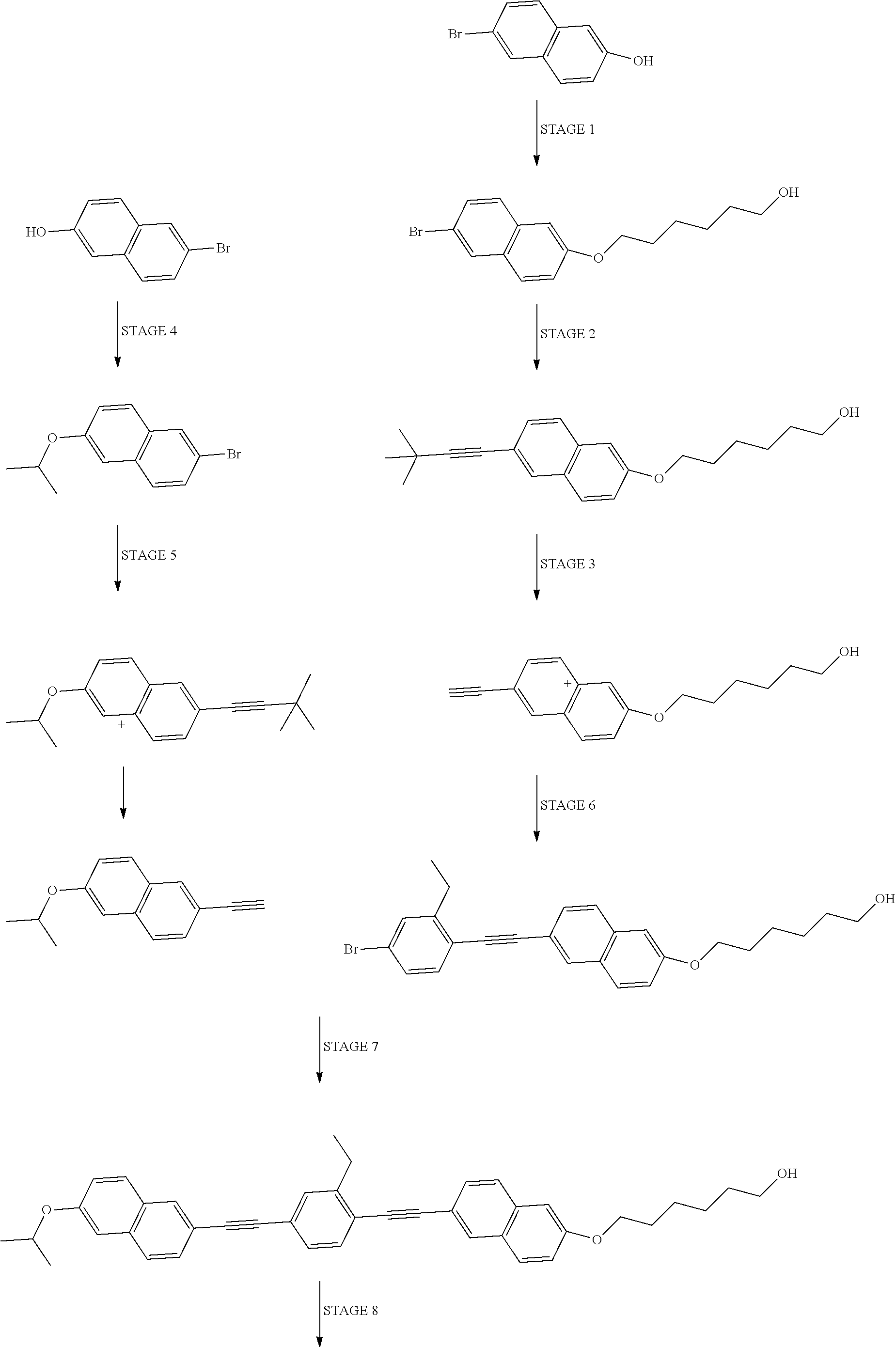

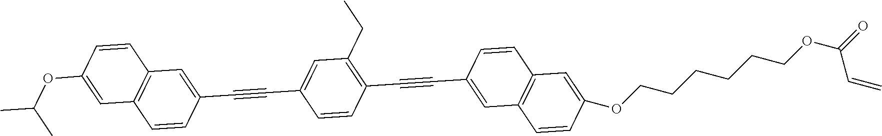 [SMILES: C=CC(=O)OCCCCCCOc1ccc2cc(C#Cc3ccc(C#Cc4ccc5cc(OC(C)C)ccc5c4)cc3CC)ccc2c1]

### Preparation of Stage 1

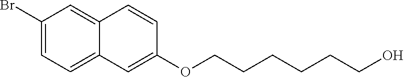 [SMILES: OCCCCCCOc1ccc2cc(Br)ccc2c1]

2-Bromo-6-hydroxynaphthalene (44.6 g, 200 mmol), potassium carbonate (33.2 g, 240 mmol), sodium iodide (6 g, 40 mmol) and butanone (125 ml) are heated to 80° C. 6-Bromohexanol (40 g, 220 mmol) is added dropwise over 1.25 hours. After 24 hours the mixture is cooled, filtered, washed with acetone and the solvent from the filtrate removed in vacuo to give product G (70 g). Product G (70 g) is dissolved in DCM (100 ml) and purified by vacuum flash chromatography on silica (220 g) eluting with the following:-DCM:ethyl acetate 200:0, 198:2, 196:4, 194:6, 192:8, 190:10, 188:12, 186:14, 184:16, 182:18, 180:20, 178:22, 176:24, 174:26 ml. Fractions 4-14 are combined and the solvent removed in vacuo to give product H (44 g). Product H (44 g) and product E (10.9 g) are combined and recrystallised toluene (100 ml) and heptane (100 ml). Cooled in the fridge for 1 hour, filtered off and washed with fridge cold toluene/petrol 1:1 then petrol to give product I (36 g). The solvent from the filtrate is removed in vacuo to give product J (18 g). Product C (22.4 g) and product I (36 g) are combined and purified by vacuum flash chromatography on silica (220 g) eluting with the following:-DCM:ethyl acetate 200:0, 200:0, 200,0, 200:0, 200:0, 198:2, 196:4, 194:6, 192:8, 190:10, 188:12, 186: 14, 184:16, 182:18, 180:20 ml. Fractions 5-14 are combined and the solvent removed in vacuo to give pure product K (55.1 g). Overall yield product F (14.5 g) plus product K (55.1 g), total 69.6 g, (54% yield).

### Preparation of Stage 2

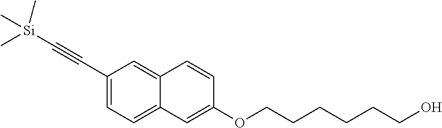 [SMILES: C[Si](C)(C)C#Cc1ccc2cc(OCCCCCCO)ccc2c1]

The reaction is carried out in two identical batches. The product of stage 1 (22 g, 68 mmol), trimethylsilylacetylene (11.5 ml, 83 mmol) and diisopropylamine (150 ml, 1.05 mol) are ultrasonicated for 30 minutes. Palladium II acetate (570 mg, 2.5 mmol), tri-tert-butylphosphonium tetrafluoroborate (644 mg, 2.2 mmol) and copper I iodide (284 mg, 1.5 mmol) are added. The mixture is slowly heated to 40° C. The reaction is exothermic, producing a thick precipitate and reached 65° C. before being cooled to 40° C. The mixture is held at 40° C. for another hour then cooled to room temperature. DCM (300 ml) then hydrochloric acid (550 ml, 2M, 1.1 mol) is added. The two layers are separated and the aqueous layer extracted with DCM (2×50 ml). The organic layers are dried over anhydrous sodium sulphate, filtered and the solvent from the filtrate removed in vacuo. Both batches are combined. The residue is dissolved in DCM silica (150 g) is added. The mixture is purified by vacuum flash chromatography on silica (300 g) eluting with the following:-DCM:ethyl acetate 200:0, 198:2, 196:4, 194:6, 192:8, 190:10, 188:12, 186:14, 184:16, 182:18, 180:20, 178:22, 176:24, 174:26, 172:28, 170:30, 168:32, 166:34, 164:36, 162:38, 160:40, 158:42, 156:44, 154:46, 152:48 150:50, 148:52, 146:54 ml. Fractions 13-23 are combined and the solvent removed in vacuo. The solid (ca. 41 g) is recrystallized from acetonitrile (80 ml), cooled in the freezer for 1 hour, filtered off and washed with freezer cold acetonitrile to give the desired product as a white solid (35.5 g, 77%).

### Preparation of Stage 3

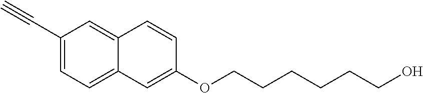 [SMILES: C#Cc1ccc2cc(OCCCCCCO)ccc2c1]

The product of stage 2 (17 g, 50 mmol) is dissolved in methanol (100 ml). Potassium carbonate (0.74 g, 5.4 mmol) is added and the mixture stirred overnight. The solvent is removed in vacuo. The residue is dissolved in DCM (100 ml) and purified by vacuum flash chromatography on silica (120 g) eluting with the following:-DCM:ethyl acetate 100:0, 95:5, 90:10, 90:10, 90:10 ml. Fractions 2-5 are combined and the solvent removed in vacuo. The solid is recrystallized from DCM (10 ml) and petrol (90 ml), cooled in the fridge for 1 hour, filtered off and washed with fridge cold petrol to give the desired product as a white solid (13.04 g, 97% yield).

### Preparation of Stage 4

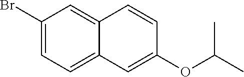

Sodium hydride (4.8 g, 120 mmol) is suspended in DMF (90 ml). 6-Bromo-2-naphthol (24 g, 108 mmol) is added in portions over 20 minutes at 10° C.-15° C. The mixture is stirred for a further 30 minutes. Isopropyl iodide (11.4 ml, 114 mmol) is added and the mixture stirred over the weekend. Water (300 ml) is cautiously added. Ethyl acetate (150 ml) and petrol (150 ml) is added and the two layers separated. The organic layer is washed with water (2×50 ml). The organic layer is dried over anhydrous sodium sulphate, filtered and the solvent from the filtrate removed in vacuo. The residue is dissolved in toluene (100 ml) and purified by vacuum flash chromatography on silica (120 g) eluting with toluene (100 ml fractions). Fractions 2-4 are combined and the solvent removed in vacuo. The residue is triturated with freezer cold petrol (100 ml) and filtered off to give the desired product as a white solid (22 g, 77% yield).

### Preparation of Stage 5

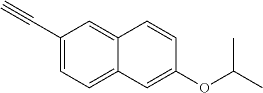 [SMILES: C#Cc1ccc2cc(OC(C)C)ccc2c1]

The product of stage 4 (22 g, 83 mmol), trimethylsilylacetylene (14.8 ml, 105 mmol) and diisopropylamine (225 ml, 1.6 mol) are ultrasonicated for 30 minutes. Palladium II acetate (210 mg, 0.94 mmol), tri-tert-butylphosphonium tetrafluoroborate (240 mg, 0.82 mmol) and copper I iodide (100 mg, 0.52 mmol) are added. The mixture is slowly heated to 45° C. The reaction is exothermic, producing a thick precipitate and reached 55° C. before being cooled to 50° C. The mixture is held at 50° C. for another hour then cooled to room temperature. DCM (300 ml) then hydrochloric acid (850 ml, 2M, 1.7 mol) are added. The two layers are separated and the aqueous layer extracted with DCM (2×100 ml). The organic layers are dried over anhydrous sodium sulphate, filtered and the solvent from the filtrate removed in vacuo. The residue is dissolved in petrol (100 ml) and silica (60 g) is added. The mixture is purified by vacuum flash chromatography on silica (60 g) eluting with the following:-petrol:ethyl acetate 100:0, 99:1, 98:2, 97:3, 96:4, 95:5, 94:6, 93:7, 92:8, 91:9, 90:10 ml. Fractions 1-9 are combined and the solvent removed in vacuo. The solid is dissolved in methanol (200 ml). Potassium carbonate (1.16 g, 8.4 mmol) is added and the mixture stirred overnight. The solvent is removed in vacuo and the residue dissolved in petrol (100 ml). Silica (60 g) is added and the mixture purified by vacuum flash chromatography on silica (60 g) eluting with the following:-petrol:ethyl acetate 200:0, 196:4, 192:8, 188:12, 184:16, 180:20, 176:24, 172:28 ml. Fractions 6-7 are combined and the solvent removed in vacuo to give the desired product as a white solid (16 g, 92% yield).

### Preparation of Stage 6

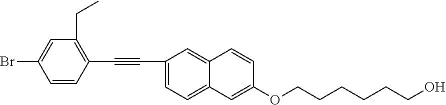 [SMILES: CCc1cc(Br)ccc1C#Cc1ccc2cc(OCCCCCCO)ccc2c1]

The product of stage 3 (17.7 g, 66 mmol), 4-bromo-2-ethyliodobenzene (20.54 g, 66 mmol), toluene (130 ml) and triethylamine (22 ml, 158 mmol) are ultrasonicated for 30 minutes. Bis(triphenylphosphine) palladium II chloride (480 mg, 0.67 mmol) and copper I iodide (200 mg, 1.05 mmol) are added. The mixture is slowly heated to 40° C. and is held at 40° C. for 2 hours then cooled to room temperature. DCM (500 ml) then hydrochloric acid (200 ml, 1M, 200 mmol) are added. The two layers are separated and the aqueous layer extracted with DCM (2×50 ml). The organic layers are dried over anhydrous sodium sulphate, filtered and the solvent from the filtrate removed in vacuo. The residue is dissolved in DCM (200 ml) and purified by vacuum flash chromatography on silica (200 g) eluting with the following:-DCM:ethyl acetate 200:0, 198:2, 196:4, 194:6, 192:8, 190:10, 188:12, 186:14, 184:16, 182:18, 180:20, 178:22, 176:24, 174:26, 172:28, 170:30, 168:32, 166:34, 164:36, 162:38, 160:40 ml. Fractions 7-19 are combined and the solvent reduced to ca. 150 ml in vacuo. Petrol (150 ml) is added, cooled in the fridge for 1 hour, filtered off and washed with fridge cold petrol to give the desired product as a white solid (20 g, 67% yield).

### Preparation of Stage 7

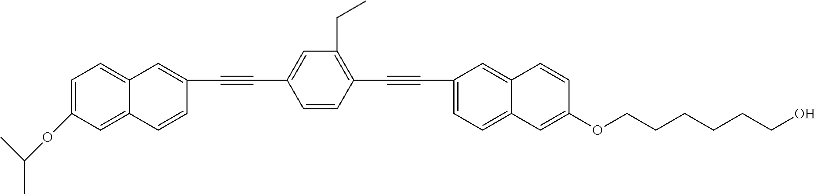 [SMILES: CCc1cc(C#Cc2ccc3cc(OC(C)C)ccc3c2)ccc1C#Cc1ccc2cc(OCCCCCCO)ccc2c1]

The products of stage 6 (20.1 g, 44.6 mmol) and stage 5 (9.77 g, 46.5 mmol) and diisopropylamine (140 ml, 1.0 mol) are ultrasonicated for 30 minutes. Palladium II acetate (320 mg, 1.4 mmol), tri-tert-butylphosphonium tetrafluoroborate (360 mg, 1.2 mmol) and copper I iodide (160 mg, 0.8 mmol) are added. The mixture is slowly heated to 45° C. The reaction is exothermic, producing a thick precipitate and reached 55° C. before being cooled to 45° C. The mixture is held at 45° C. for 1.5 hour then cooled to room temperature. DCM (500 ml) then hydrochloric acid (500 ml, 2M, 1 mol) is added. The two layers are separated and the aqueous layer extracted with DCM (2×50 ml). The organic layers are dried over anhydrous sodium sulphate, filtered and the solvent from the filtrate removed in vacuo. The residue is dissolved in DCM (200 ml) and silica (100 g) is added. The mixture is purified by vacuum flash chromatography on silica (100 g) eluting with the following:-DCM:ethyl acetate 200:0, 198:2, 196:4, 194:6, 192:8, 190:10, 188:12, 186:14, 184:16, 182:18, 180:20, 178:22, 176:24, 174:26, 172:28, 170:30, 168:32, 166:34, 164:36, 162:38, 160:40 ml. Fractions 3-21 are combined and the solvent removed in vacuo. The solid is recrystallized from DCM (40 ml) and petrol (160 ml), cooled in the fridge for 1 hour, filtered off and washed with fridge cold petrol to give the desired product as a white solid (24.73 g, 96% yield).

### Preparation of Stage 8

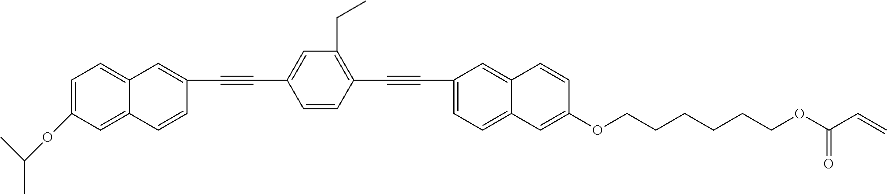 [SMILES: C=CC(=O)OCCCCCCOc1ccc2cc(C#Cc3ccc(C#Cc4ccc5cc(OC(C)C)ccc5c4)cc3CC)ccc2c1]

Alcohol (APN 2187, 24.1 g, 41.6 mmol), triethylamine (25 ml, 178 mmol) and DCM (250 ml) are stirred in an ice bath. 3-Chloropropionyl chloride (4.7 ml, 49.2 mmol) in DCM (10 ml) is added dropwise over 20 minutes. The mixture is stirred for a further 30 minutes. Additional triethylamine (75 ml, 539 mmol) is added, heated to 35° C. for 18 hours, then cooled to room temperature. DCM (1000 ml) is added then hydrochloric acid (360 ml, 2M, 720 mmol) is added to the reaction mixture. The two layers are separated and the aqueous layer extracted with DCM (2×250 ml). The organic layers are dried over anhydrous sodium sulphate, filtered and the solvent from the filtrate removed in vacuo. The residue is dissolved in DCM (100 ml) and purified by vacuum flash chromatography on silica (220 g) eluting with DCM (200 ml fractions). Fractions 2-6 are combined and the solvent removed in vacuo. The solid is recrystallized three times from DCM (60 ml) and petrol (240 ml), cooled in the fridge for 1 hour, filtered off and washed with fridge cold petrol to give the desired product as a white solid (23.07 g, 88% yield).

The compound shows the phase transitions K79N163 I.

Compound (RM-46) is prepared in accordance with the following scheme:

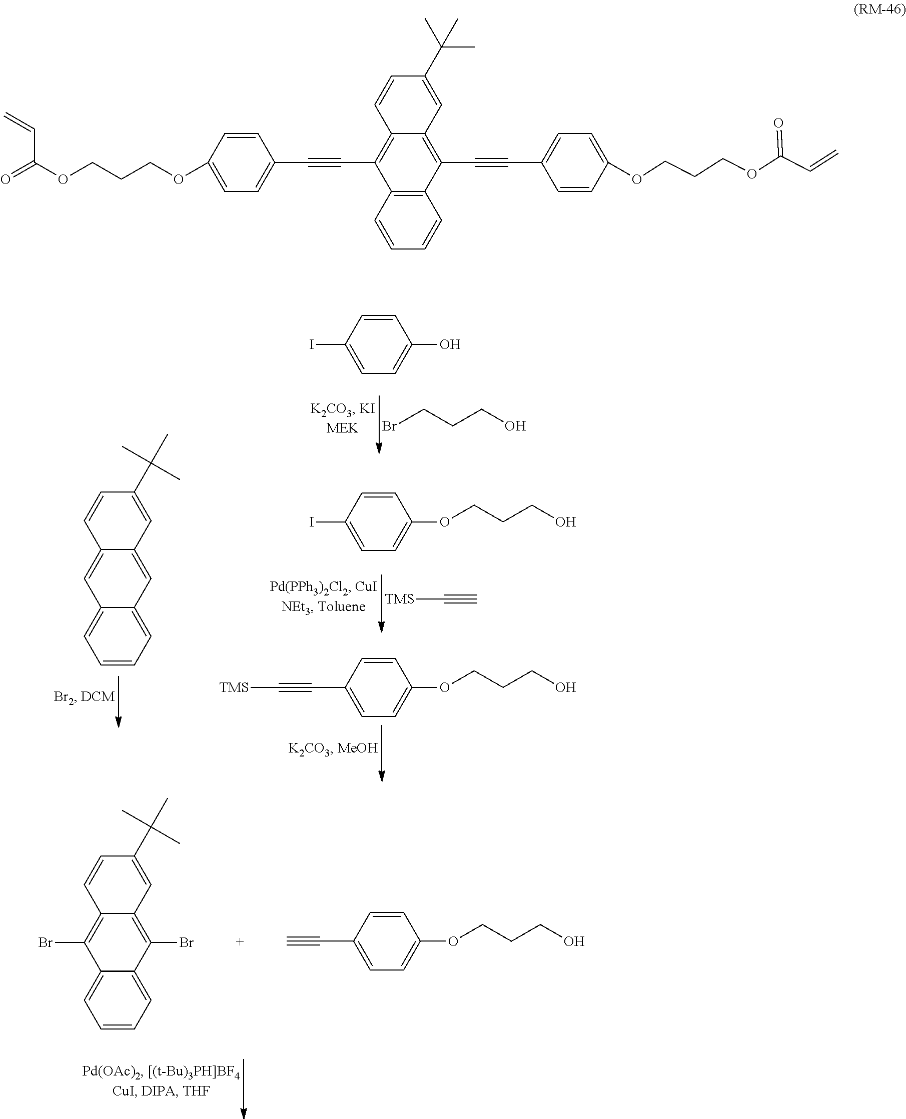

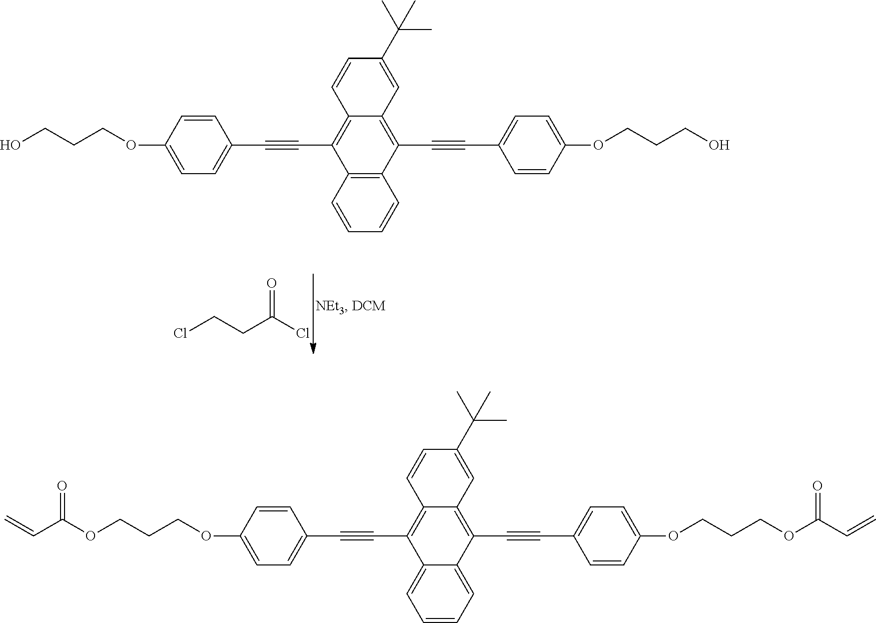 [SMILES: CC(C)(C)c1ccc2c(C#Cc3ccc(OCCCO)cc3)c3ccccc3c(C#Cc3ccc(OCCCO)cc3)c2c1 ; O=C(Cl)CCCl ; C=CC(=O)OCCCOc1ccc(C#Cc2c3ccccc3c(C#Cc3ccc(OCCCOC(=O)C=C)cc3)c3cc(C(C)(C)C)ccc23)cc1]

### Preparation of Stage 1

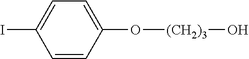

4-Iodophenol (66 g, 300 mmol), potassium carbonate (52.2 g, 378 mmol), 3-bromopropanol (45 g, 323 mmol), sodium iodide (0.3 g, 2 mmol) and butanone (100 ml) are heated to 80° C. overnight. The mixture is cooled, filtered, washed with acetone and the solvent from the filtrate is removed in vacuo. The oil is crystallized from petrol (250 ml), cooled in the fridge for 1 hour, filtered off and washed with fridge cold petrol to give the product as a solid (77.9 g, 93% yield).

### Preparation of Stage 2

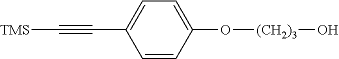

The product of stage 1 (75 g, 270 mmol), trimethylsilylacetylene (45 ml, 326 mmol), triethylamine (60 ml, 431 mmol) and toluene (360 ml) are ultrasonicated for 15 minutes. Copper(I)iodide (0.54 g, 2.8 mmol), bis(triphenylphosphine)palladium(II)chloride (1.08 g, 1.5 mmol) are added and the mixture stirred at room temperature. The reaction is exothermic and reached 35° C. After the temperature had stabilized, the mixture is heated to 40° C., held for 2 hours then cooled to room temperature. Hydrochloric acid (250 ml, 2M, 0.5 mol) is added. The two layers are separated and the aqueous layer extracted with toluene (50 ml). Silica (200 g) is added to the combined filtrates. The mixture is purified by vacuum flash chromatography on silica (200 g) eluting with the following:-DCM:ethyl acetate 500:0, 1000:0, 980:20, 960:40, 940:60, 920:80 ml. Fractions 3-6 are combined and the solvent removed in vacuo to give the product as an orange oil (55 g, 82%).

### Preparation of Stage 3

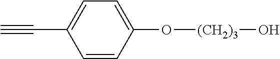

The product of stage 2 (54.6 g, 220 mmol) is dissolved in methanol (300 ml). Potassium carbonate (3.03 g, 22 mmol) is added and the mixture stirred for 1 hour at 40° C. The solvent is removed in vacuo. DCM (200 ml) and Silica (150 g) are added to the residue. The mixture is purified by vacuum flash chromatography on silica (200 g) eluting with the following:-DCM:ethyl acetate 1000:0, 980:20, 960:40, 940:60, 920:80, 900:100, 880:120 ml. Fractions 2-7 are combined and the solvent removed in vacuo. The oil is triturated with petrol (100 ml), cooled in the fridge for 1 hour, filtered off and washed with fridge cold petrol to give the product as a solid (36.7 g, 95% yield).

### Preparation of Stage 4

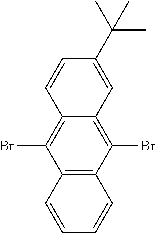 [SMILES: CC(C)(C)c1ccc2c(Br)c3ccccc3c(Br)c2c1]

2-tert-Butylanthracene (2.3 g, 9.8 mmol) and DCM (50 ml) are cooled in an ice bath. Bromine (3.2 g, 20 mmol), dissolved in DCM (50 ml) is added dropwise over 1.5 hours at 5° C. The mixture is warmed to room temperature for 16 hours. Water (50 ml) is added. The two layers are separated and the organic layer washed with saturated sodium bicarbonate. The organic layer is dried over anhydrous sodium sulphate, filtered and the solvent from the filtrate removed in vacuo. The solid is dissolved in DCM (50 ml). IMS (75 ml) is added and the mixture boiled to remove the DCM. The mixture is left in the fridge for 1 hour. The solid is filtered off and washed with fridge cold IMS to give the desired product (3.4 g, 88% yield).

### Preparation of Stage 5

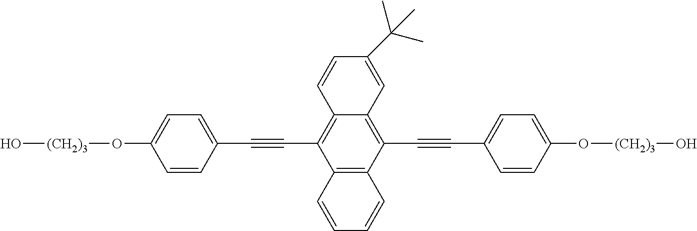

The products of stage 4 (1.7 g, 4.3 mmol) and stage 3 (1.7 g, 9.7 mmol), THF (5 ml) and diisopropylamine (25 ml, 178 mmol) are ultrasonicated for 30 minutes. Palladium II acetate (90 mg, 0.40 mmol), tri-tert-butylphosphonium tetrafluoroborate (102 mg, 0.35 mmol) and copper I iodide (45 mg, 0.24 mmol) are added. The mixture is very slowly heated to 60° C. then held for a further 1.5 hours, producing a thick precipitate. The mixture is cooled, diluted with water (35 ml) and acidified with concentrated hydrochloric acid (15 ml, 11.6M, 174 mmol). The mixture is extracted with DCM (500 ml and 2 ×50 ml). The solvent from the combined organic extracts is removed in vacuo. The residue is dissolved in DCM (100 ml). Silica (40 g) is added and the mixture is purified by vacuum flash chromatography on silica (40 g) eluting with the following:-DCM:ethyl acetate: IPA 200:0:0, 198:2:0, 196:4:0, 194:6:0, 192:8:0, 190:10:0, 188: 10:2, 186:10:4, 184:10:6, 182:10:8, 180:10:10, 178: 10: 12 ml. Fractions 11-12 are combined and the solvent reduced to give the product. (1.71 g, 68% yield).

### Preparation of Stage 6

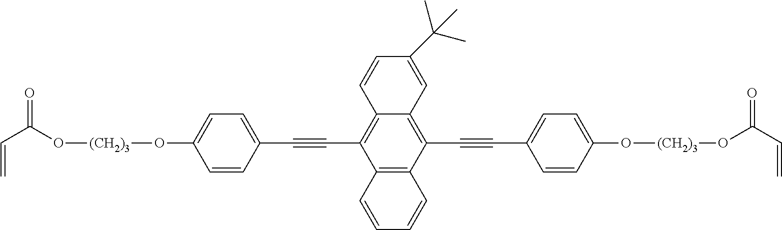

The product of stage 5 (1.71 g, 2.9 mmol), DCM (30 ml) and 3-chloropropionyl chloride (0.8 ml, 8.4 mmol) are stirred on an ice bath. Triethylamine (3 ml, 22 mmol) in DCM (10 ml) is added dropwise over 20 minutes. Additional triethylamine (17 ml, 122 mmol) is added over 10 minutes. The mixture is allowed to warm to room temperature and held at room temperature for 30 minutes. The mixture is heated to 35° C. for 18 hours, then cooled to room temperature. DCM (150 ml) and hydrochloric acid (80 ml, 2M, 160 mmol) are added. The two layers are separated and the aqueous layer extracted with DCM (2×50 ml). The solvent from the combined extracts are removed in vacuo. The residue is dissolved in DCM (100 ml) and silica (40 g) is added. The mixture is purified by vacuum flash chromatography on silica (80 g) eluting with DCM (150 ml fractions). Fractions 5-8 are combined and the solvent reduced to 20 ml. Petrol (100 ml) is added and cooled in the fridge for 1 hour. The solid is filtered off and washed with fridge cold petrol to give the product (1.16 g, 57% yield, HPLC analysis 99.7%).

The compound shows the phase transitions K155I.

Compound (RM-59) is prepared in accordance with the following scheme:

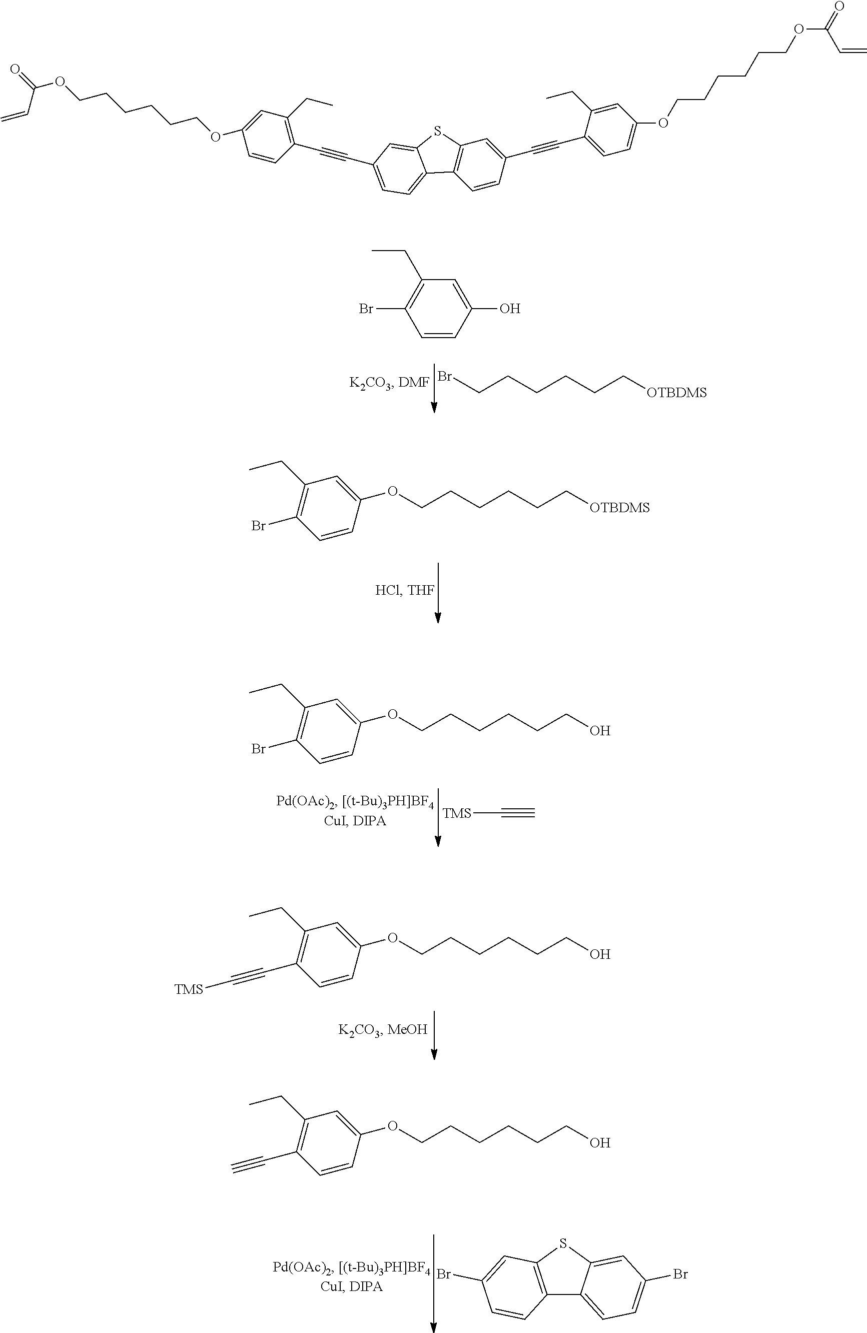

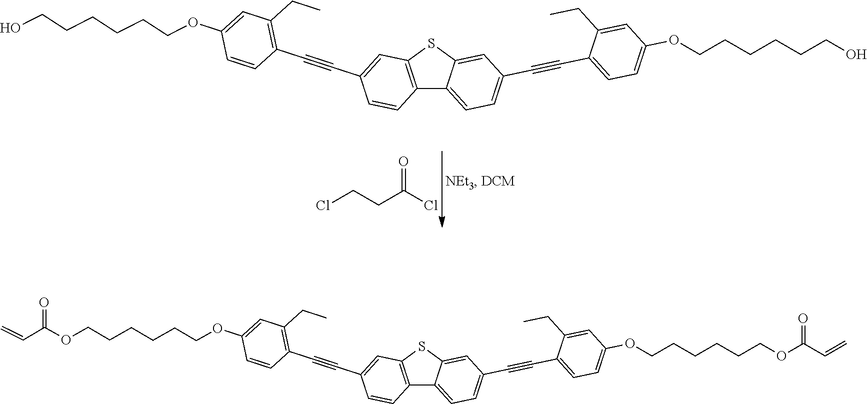 [SMILES: CCc1cc(OCCCCCCO)ccc1C#Cc1ccc2c(c1)sc1cc(C#Cc3ccc(OCCCCCCO)cc3CC)ccc12 ; O=C(Cl)CCCl ; C=CC(=O)OCCCCCCOc1ccc(C#Cc2ccc3c(c2)sc2cc(C#Cc4ccc(OCCCCCCOC(=O)C=C)cc4CC)ccc23)c(CC)c1]

All reactions carried out under nitrogen unless otherwise stated. Petroleum ether=40-60° C. fraction

### Preparation of Stage 1

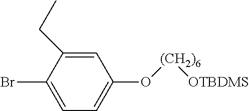

A mixture of 6-bromo-1-(tert-butyldimethylsilyloxy)hexane (7.2 g; 0.024 moles), 4-bromo-3-ethylphenol (4.9 g; 0.024 moles) and potassium carbonate (7.4 g; 0.054 moles) is stirred in anhydrous DMF (80 ml) at 80° C. for 16 hours. After cooling the mixture is partitioned between ethyl acetate/petroleum ether and water. The separated organic layer is washed with water, brine, dried over sodium sulphate and evaporated in vacuo. The residue is dissolved in petroleum ether and layered onto silica (125 g) eluting with 5% ethyl acetate in petroleum ether to afford the product as a colourless oil (7.7 g; 76%).

### Preparation of Stage 2

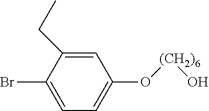

To a solution of the product of stage 1 (7.7 g; 0.019 moles) in THF (150 ml) is added 2M HCl (50 ml). The resulting solution is stirred for 75 minutes then the excess organic solvent is removed in vacuo and the mixture partitioned between ethyl acetate and brine, dried over sodium sulphate and evaporated in vacuo to afford the product as a mixture with tert-butyldimethylsilanol (8 g yield overall; assume quantitative yield as ˜ 5.3 g) which is used directly in the next step.

### Preparation of Stage 3

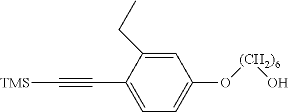

To a degassed solution of the product of stage 2 (8.0 g crude; assume 5.3 g pure; 0.0186 moles) and TMS acetylene (2.36 g; 0.024 moles) in diisopropylamine (50 ml) is added copper I iodide (44 mg), tri-(tert-butylphosphonium)tetrafluoroborate (109 mg) then palladium II acetate (89 mg). The mixture is heated to 45° C. for 2 hours, cooled, evaporated in vacuo, slurried in ethyl acetate then filtered and the filtrate evaporated in vacuo then azeotroped with petroleum ether. The residue is chromatographed on silica (125 g) eluting with 33% petroleum ether in DCM followed by 5% ethyl acetate in DCM to afford the product as a brown oil (5.4 g).

This product is not analysed by NMR but pushed on to the next reaction, where it transpired that there is a 40% impurity by NMR comparison, most likely also present in this precursor product.

### Preparation of Stage 4

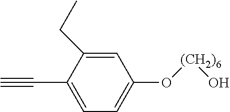

To a solution of the product of stage 3 (5.4 g; 0.018 moles) in methanol (150 ml) is added potassium carbonate (0.25 g; 0.0018 moles. The mixture is stirred at ambient temperature overnight then evaporated in vacuo, azeotroped with DCM and petroleum ether then layered in DCM onto silica (90 g) eluting with DCM then ethyl acetate to afford the product as an orange oil (4.2 g).

NMR analysis indicated this is an inseparable circa 40% impurity, which is removed in the next reaction. The material is assumed to be 50% pure to allow for a safe excess in the next step.

### Preparation of Stage 5

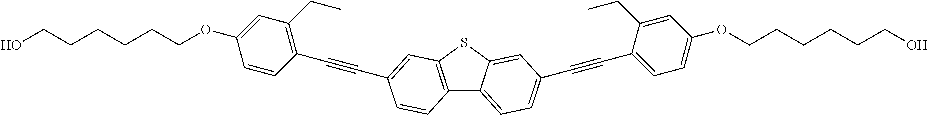 [SMILES: CCc1cc(OCCCCCCO)ccc1C#Cc1ccc2c(c1)sc1cc(C#Cc3ccc(OCCCCCCO)cc3CC)ccc12]

To a degassed solution of the product of stage 4 (4.2 g crude; assume 2.1 g pure; 0.008 moles) and 3,7-dibromodibenzothiophene (1.37 g; 0.004 moles) in diisopropylamine (25 ml) is added copper I iodide (47 mg), tri-(tert-butylphosphonium)tetrafluoroborate (121 mg) then palladium II acetate (96 mg). The mixture is heated to 45° C. for 2 hours, cooled, evaporated in vacuo, then stirred in a mixture of ethyl acetate and 2M hydrochloric acid, filtered then washed with ethyl acetate and water to afford the product as an off-white solid after drying in vacuo at 50° C. (1.9 g; 71%).

### Preparation of Stage 6

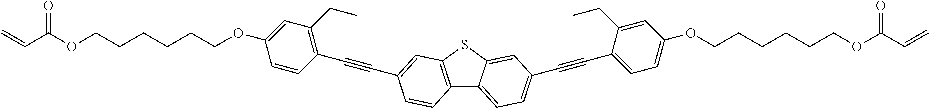 [SMILES: C=CC(=O)OCCCCCCOc1ccc(C#Cc2ccc3c(c2)sc2cc(C#Cc4ccc(OCCCCCCOC(=O)C=C)cc4CC)ccc23)c(CC)c1]

To an ice/water bath-cooled slurry of the product of stage 5 (1.9 g; 0.0028 moles) in anhydrous DCM (600 ml) and triethylamine (1.25 ml; 0.009 moles) is added dropwise over 15 minutes 3-chloropropionyl chloride (0.94 g; 0.0074 moles). After 30 minutes, further triethylamine (2.5 ml) is added dropwise over 10 minutes. After 15 minutes more trimethylamine is added (3 ml) over 5 minutes then 3-chloropropionyl chloride (0.35 ml; 0.0037 moles) is added over 5 minutes. The reaction mixture is allowed to warm to room temperature for 20 minutes. TLC analysis of the opaque mixture indicated still a trace of starting material so further trimethylamine (3 ml) then 3-chloropropionyl chloride (0.35 ml) are added sequentially at room temperature which quickly resulted in a solution. After 20 minutes, trimethylamine (30 ml) and some Irganox are added and the solution heated to 38° C. overnight, cooled and evaporated in vacuo. The residue is dissolved in DCM (100 ml), petroleum ether (50 ml) added and the mixture filtered and washed with 2:1 DCM:petroleum ether. The filtrate is evaporated in vacuo then re-dissolved in DCM, washed with 2M hydrochloric acid, dried over sodium sulphate and evaporated in vacuo.

The residue is dissolved in DCM (30 ml), petroleum ether added (10 ml) then layered onto silica (60 g) eluting with 50-100% DCM in petroleum ether to give the crude product as a cream/white solid. This is dissolved in DCM (25 ml), petroleum ether (100 ml) added, left in the freezer for 30 minutes then filtered and washed with 4:1 petroleum ether: DCM then petroleum ether to afford the product as a white solid after drying in vacuo at 30° C. (1.3 g; 59%).

The compound shows the phase transitions K130N160I.

Compound (RM-57) is prepared in accordance with the following scheme:

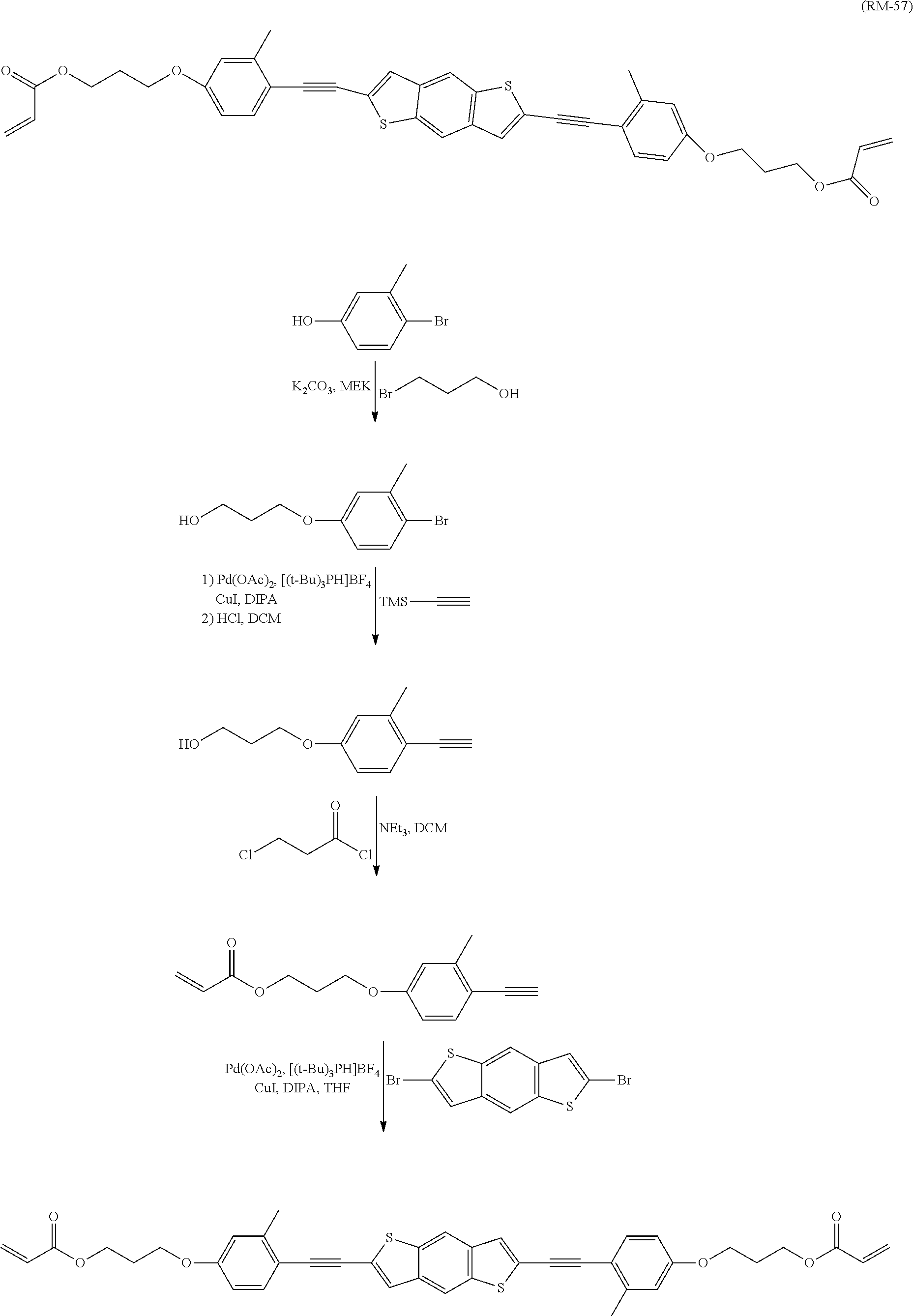

### Preparation of Stage 1

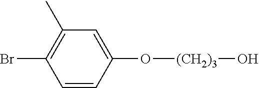

4-Bromo-3-methylphenol (18.7 g, 100 mmol), 3-bromopropanol (15.3 g, 110 mmol), potassium carbonate (16.5 g, 120 mmol) and butanone (50 ml) are heated to 80° C. for 2.5 hours. Additional 3-bromopropanol (1.8 g, 12.9 mmol), potassium carbonate (1.8 g, 13 mmol) are added and heated at 80° C. for a further 3.5 hours. The mixture is cooled, filtered, washed with acetone and the solvent from the filtrate removed in vacuo (24.5 g, 100% yield).

### Preparation of Stage 2

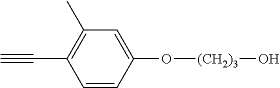

The product of stage 1 (24.5 g, 100 mmol), trimethylsilylacetylene (18 ml, 128 mmol) and diisopropylamine (270 ml, 1.94 mol) are ultrasonicated for 30 minutes. Palladium II acetate (240 mg, 1.07 mmol), tri-tert-butylphosphonium tetrafluoroborate (280 mg, 0.97 mmol) and copper I iodide (120 mg, 0.63 mmol) are added. The mixture is slowly heated to 55° C. (no exotherm, reaction slow). The mixture is cooled to 30° C. Additional palladium II acetate (240 mg, 1.07 mmol), tri-tert-butylphosphonium tetrafluoroborate (280 mg, 0.97 mmol) and copper I iodide (120 mg, 0.63 mmol) are added. The mixture is slowly heated to 45° C. and held at 45° C. for 6 hours. Additional trimethylsilylacetylene (18 ml, 128 mmol), palladium II acetate (480 mg, 2.14 mmol), tri-tert-butylphosphonium tetrafluoroborate (560 mg, 1.93 mmol) and copper I iodide (240 mg, 1.26 mmol) are added then heated to 55° C. for a further 6 hours. The mixture is cooled to room temperature. Hydrochloric acid (1000 ml, 2M, 2 mol) is added. DCM (250 ml) is added, the two layers are separated and the aqueous layer extracted with DCM (2×100 ml). The combined organic layers are dried over anhydrous sodium sulphate, filtered and the solvent from the filtrate removed in vacuo. The residue is dissolved in DCM (100 ml) and silica (60 g) is added. The mixture is purified by vacuum flash chromatography on silica (140 g) eluting with the following:-DCM:ethyl acetate 200:0, 196:4, 192:8, 188:12, 184:16, 180:20, 176:24, 172:28, 168:32, 164:36, 160:40 ml. Fractions 3-9 are combined and the solvent removed in vacuo (24 g). The oil (24 g) is dissolved in petrol (80 ml) and purified by vacuum flash chromatography on silica (80 g) eluting with the following:-Petrol:ethyl acetate 100:0, 95:5, 90:10, 85:15, 80:20, 75:25, 70:30, 65:35, 60:40, 55:45, 50:50 ml. Fractions 6-10 are combined and the solvent removed in vacuo (16.5 g). The oil (16.5 g) is dissolved in methanol (120 ml). Potassium carbonate (0.87 g, 6.3 mmol) is added and the mixture stirred overnight at room temperature. The solvent is removed in vacuo. The residue is suspended in a mixture of ethyl acetate (20 ml) and petrol (80 ml). The mixture is purified by vacuum flash chromatography on silica (80 g) eluting with the following:-Petrol:ethyl acetate 100:0, 95:5, 90:10, 85:15, 80:20, 75:25, 70:30, 65:35, 60:40, 55:45, 50:50 ml. Fractions 5-9 are combined and the solvent removed in vacuo to give the desired product (6 g, 32% yield).

### Preparation of Stage 3

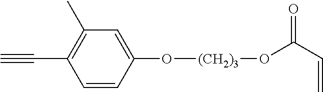

The product of stage 2 (6 g, 31.6 mmol), triethylamine (15 ml, 108 mmol) and DCM (100 ml) are stirred in an ice bath. 3-Chloropropionyl chloride (3.4 ml, 35.4 mmol) in DCM (10 ml) is added dropwise over 15 minutes. The mixture is stirred for a further 30 minutes. Additional triethylamine (30 ml, 216 mmol) is added, heated to 35° C. for 18 hours, then cooled to room temperature. The mixture is acidified with hydrochloric acid (150 ml, 2M, 300 mmol). The two layers are separated and the aqueous layer extracted with DCM (2×25 ml). The combined organic layers are dried over anhydrous sodium sulphate, filtered and the solvent from the filtrate removed in vacuo. The residue is dissolved in DCM (100 ml) and purified by vacuum flash chromatography on silica (120 g) eluting with DCM (100 ml) fractions. Fractions 2-5 are combined to give the desired product (7 g, 91% yield).

### Preparation of Stage 4

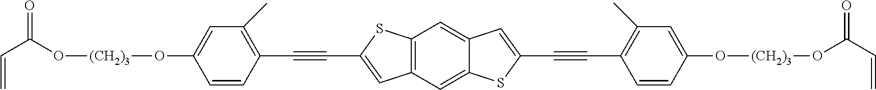

The product of stage 3 (5.3 g, 21.7 mmol), 2,6-dibromo[1,2-b:4,5-b]dithiophene (3.48 g, 10 mmol), THF (80 ml) and diisopropylamine (40 ml, 0.35 mol) are ultrasonicated for 15 minutes. Palladium II acetate (240 mg, 0.89 mmol), tri-tert-butylphosphonium tetrafluoroborate (270 mg, 0.78 mmol) and copper I iodide (120 mg, 0.52 mmol) are added. The mixture is slowly heated to 55° C. and held at 55° C. for 1 hour before cooling to room temperature. DCM (300 ml) is added and the mixture purified by vacuum flash chromatography on silica (100 g) eluting with DCM (500 ml). The solvent volume is reduced to 40 ml. Petrol (40 ml) is added, cooled in the fridge for 1 hour, filtered off and washed with fridge cold petrol to give the desired product (5.94 g, 85% yield). The product (4 g) is further purified by dissolving in boiling DCM (200 ml). Silica (40 g) is added and the mixture purified by vacuum flash chromatography on silica (80 g) eluting with DCM (200 ml) fractions. Fractions 3-6 are combined. The solvent volume is reduced to 50 ml. Petrol (50 ml) is added, cooled in the fridge for 1 hour, filtered off and washed with fridge cold petrol to give the desired product as a lemon yellow solid (3.2 g, 80% recovery).

The compound shows the phase transitions K174N2291

### Compound Examples

The following compounds are prepared by the synthesis methods described above or in analogy thereto.

| | Transition |
|---|---|
| | Temperatures |
| Structure | in ° C. |
| 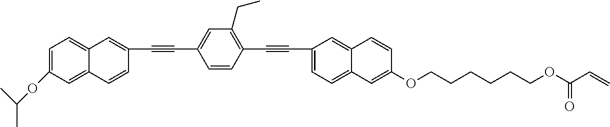 [SMILES: C=CC(=O)OCCCCCCOc1ccc2cc(C#Cc3ccc(C#Cc4ccc5cc(OC(C)C)ccc5c4)cc3CC)ccc2c1] | K79N163 I |
| RM-1 | |
| 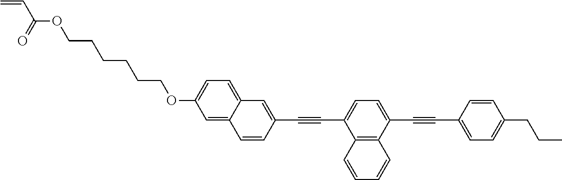 [SMILES: C=CC(=O)OCCCCCCOc1ccc2cc(C#Cc3ccc(C#Cc4ccc(CCC)cc4)c4ccccc34)ccc2c1] | K46N146Sm 209I |
| RM-2 | |
| 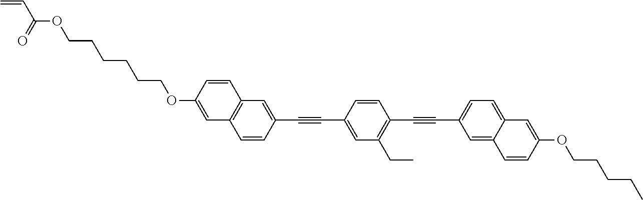 [SMILES: C=CC(=O)OCCCCCCOc1ccc2cc(C#Cc3ccc(C#Cc4ccc5cc(OCCCCC)ccc5c4)c(CC)c3)ccc2c1] | K94N234I |
| RM-3 | |
| 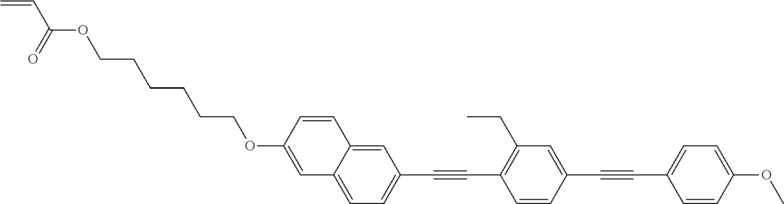 [SMILES: C=CC(=O)OCCCCCCOc1ccc2cc(C#Cc3ccc(C#Cc4ccc(OC)cc4)cc3CC)ccc2c1] | K86N192I |
| RM-4 | |
| 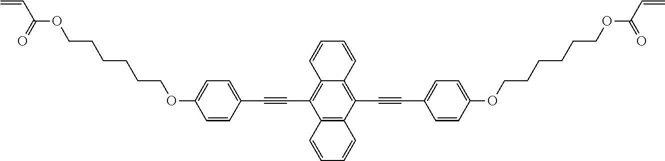 [SMILES: C=CC(=O)OCCCCCCOc1ccc(C#Cc2c3ccccc3c(C#Cc3ccc(OCCCCCCOC(=O)C=C)cc3)c3ccccc23)cc1] | K141I |
| RM-5 | |
| 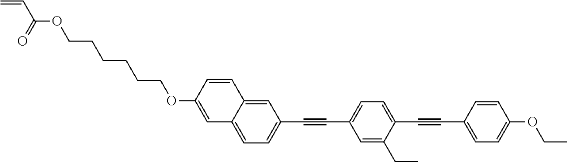 [SMILES: C=CC(=O)OCCCCCCOc1ccc2cc(C#Cc3ccc(C#Cc4ccc(OCC)cc4)c(CC)c3)ccc2c1] | K98N201I |
| RM-6 | |
| 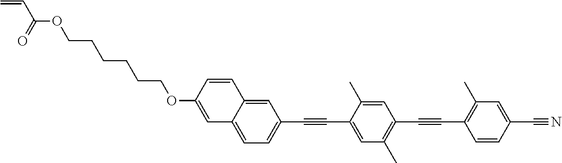 [SMILES: C=CC(=O)OCCCCCCOc1ccc2cc(C#Cc3cc(C)c(C#Cc4ccc(C#N)cc4C)cc3C)ccc2c1] | K109N211I |
| RM-7 | |
| 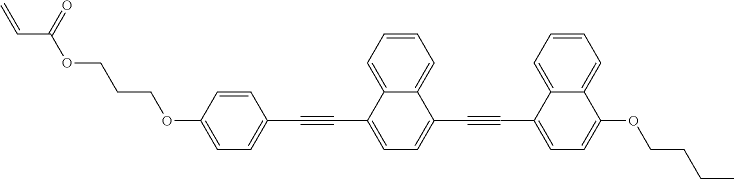 [SMILES: C=CC(=O)OCCCOc1ccc(C#Cc2ccc(C#Cc3ccc(OCCCC)c4ccccc34)c3ccccc23)cc1] | K111N180I |
| RM-8 | |
| 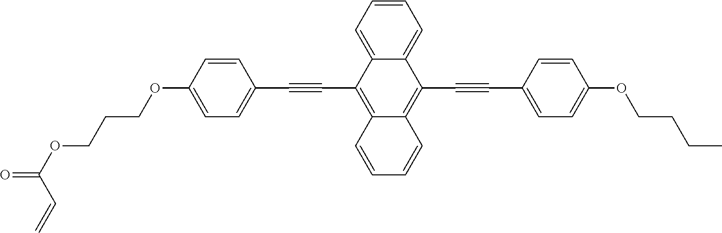 [SMILES: C=CC(=O)OCCCOc1ccc(C#Cc2c3ccccc3c(C#Cc3ccc(OCCCC)cc3)c3ccccc23)cc1] | K166N178I |
| RM-9 | |
| 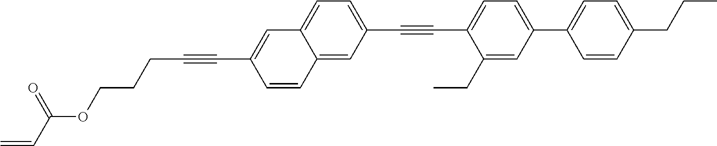 [SMILES: C=CC(=O)OCCCC#Cc1ccc2cc(C#Cc3ccc(-c4ccc(CCC)cc4)cc3CC)ccc2c1] | K62N154I |
| RM-10 | |
| 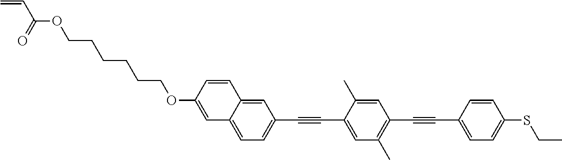 [SMILES: C=CC(=O)OCCCCCCOc1ccc2cc(C#Cc3cc(C)c(C#Cc4ccc(SCC)cc4)cc3C)ccc2c1] | K121N140X 186I |
| RM-11 | |
| 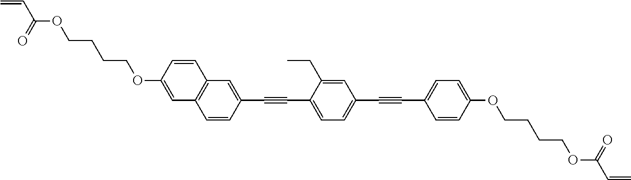 [SMILES: C=CC(=O)OCCCCOc1ccc(C#Cc2ccc(C#Cc3ccc4cc(OCCCCOC(=O)C=C)ccc4c3)c(CC)c2)cc1] | K106N230I |
| RM-12 | |
| 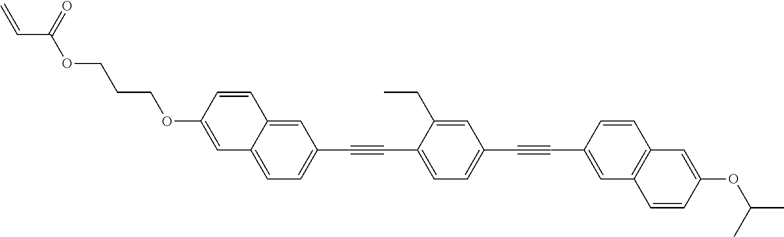 [SMILES: C=CC(=O)OCCCOc1ccc2cc(C#Cc3ccc(C#Cc4ccc5cc(OC(C)C)ccc5c4)cc3CC)ccc2c1] | K131I |
| RM-13 | |
| 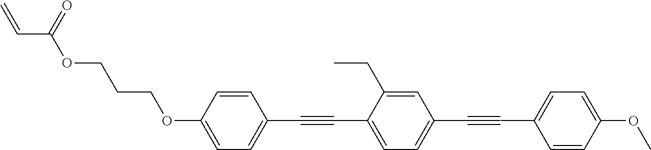 [SMILES: C=CC(=O)OCCCOc1ccc(C#Cc2ccc(C#Cc3ccc(OC)cc3)cc2CC)cc1] | K113N158I |
| RM-14 | |
| 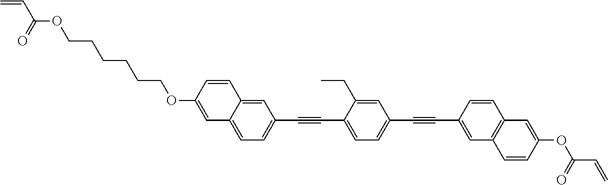 [SMILES: C=CC(=O)OCCCCCCOc1ccc2cc(C#Cc3ccc(C#Cc4ccc5cc(OC(=O)C=C)ccc5c4)cc3CC)ccc2c1] | K107(N97)I |
| RM-15 | |
| 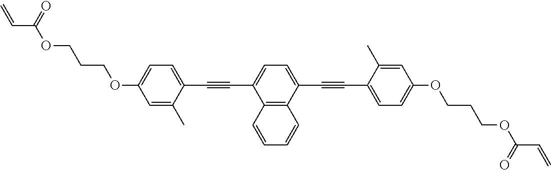 [SMILES: C=CC(=O)OCCCOc1ccc(C#Cc2ccc(C#Cc3ccc(OCCCOC(=O)C=C)cc3C)c3ccccc23)c(C)c1] | K90N140I |
| RM-16 | |
| 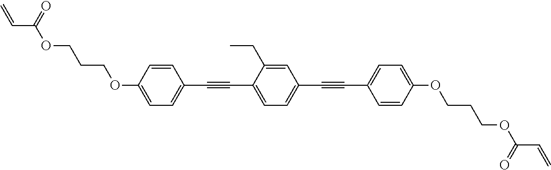 [SMILES: C=CC(=O)OCCCOc1ccc(C#Cc2ccc(C#Cc3ccc(OCCCOC(=O)C=C)cc3)c(CC)c2)cc1] | K85SmX111N1 26I |
| RM-17 | |
| 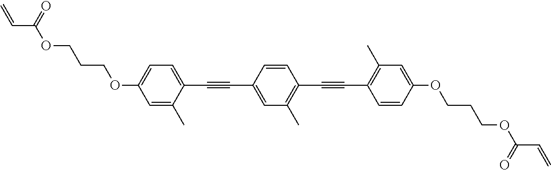 [SMILES: C=CC(=O)OCCCOc1ccc(C#Cc2ccc(C#Cc3ccc(OCCCOC(=O)C=C)cc3C)c(C)c2)c(C)c1] | |
| RM-18 | |
| 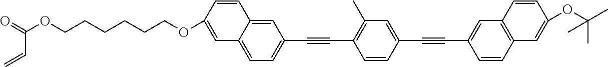 [SMILES: C=CC(=O)OCCCCCCOc1ccc2cc(C#Cc3ccc(C#Cc4ccc5cc(OC(C)(C)C)ccc5c4)cc3C)ccc2c1] | K158N238I |
| RM-19 | |
| 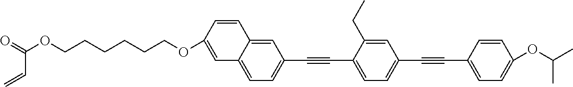 [SMILES: C=CC(=O)OCCCCCCOc1ccc2cc(C#Cc3ccc(C#Cc4ccc(OC(C)C)cc4)cc3CC)ccc2c1] | K79N163I |
| RM-20 | |
| 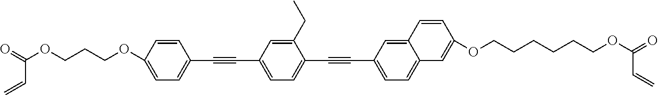 [SMILES: C=CC(=O)OCCCCCCOc1ccc2cc(C#Cc3ccc(C#Cc4ccc(OCCCOC(=O)C=C)cc4)cc3CC)ccc2c1] | K84N165I |
| RM-21 | |
| 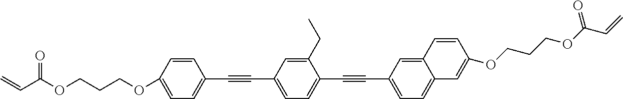 [SMILES: C=CC(=O)OCCCOc1ccc(C#Cc2ccc(C#Cc3ccc4cc(OCCCOC(=O)C=C)ccc4c3)c(CC)c2)cc1] | K129K143N26 0I |
| RM-22 | |
| 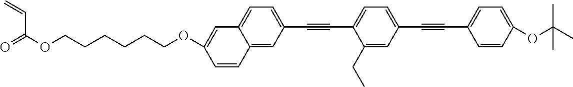 [SMILES: C=CC(=O)OCCCCCCOc1ccc2cc(C#Cc3ccc(C#Cc4ccc(OC(C)(C)C)cc4)cc3CC)ccc2c1] | K86N125I |
| RM-23 | |
| 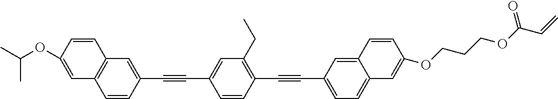 [SMILES: C=CC(=O)OCCCOc1ccc2cc(C#Cc3ccc(C#Cc4ccc5cc(OC(C)C)ccc5c4)cc3CC)ccc2c1] | K132N260I |
| RM-24 | |
| 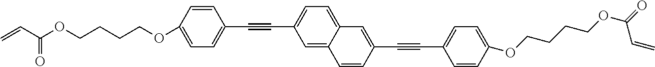 [SMILES: C=CC(=O)OCCCCOc1ccc(C#Cc2ccc3cc(C#Cc4ccc(OCCCCOC(=O)C=C)cc4)ccc3c2)cc1] | K188N270I |
| RM-25 | |
| 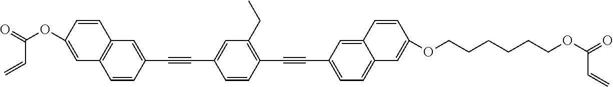 [SMILES: C=CC(=O)OCCCCCCOc1ccc2cc(C#Cc3ccc(C#Cc4ccc5cc(OC(=O)C=C)ccc5c4)cc3CC)ccc2c1] | K73N245I |
| RM-26 | |
| 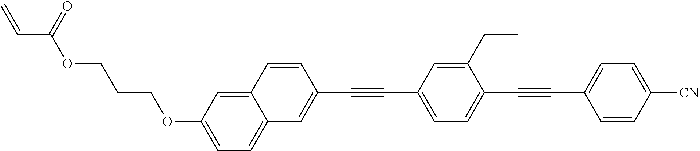 [SMILES: C=CC(=O)OCCCOc1ccc2cc(C#Cc3ccc(C#Cc4ccc(C#N)cc4)c(CC)c3)ccc2c1] | K131N197I |
| RM-27 | |
| 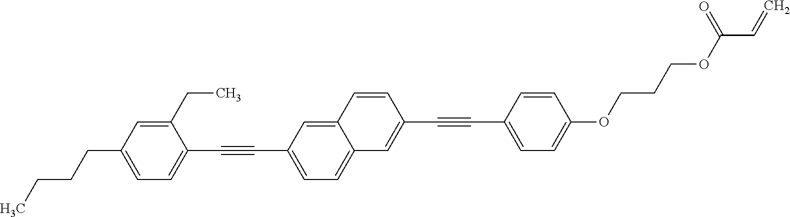 [SMILES: C=CC(=O)OCCCOc1ccc(C#Cc2ccc3cc(C#Cc4ccc(CCCC)cc4CC)ccc3c2)cc1] | K99N187I |
| RM-28 | |
| 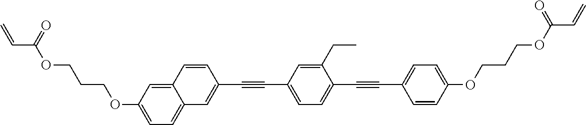 [SMILES: C=CC(=O)OCCCOc1ccc(C#Cc2ccc(C#Cc3ccc4cc(OCCCOC(=O)C=C)ccc4c3)cc2CC)cc1] | K92N187I |
| RM-29 | |
| 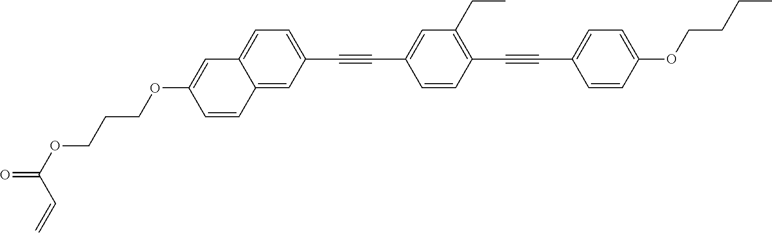 [SMILES: C=CC(=O)OCCCOc1ccc2cc(C#Cc3ccc(C#Cc4ccc(OCCCC)cc4)c(CC)c3)ccc2c1] | K92N215I |
| RM-30 | |
| 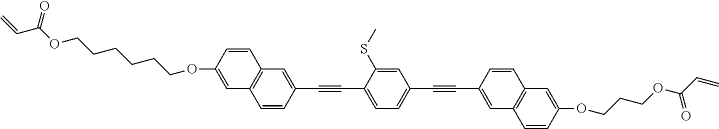 [SMILES: C=CC(=O)OCCCCCCOc1ccc2cc(C#Cc3ccc(C#Cc4ccc5cc(OCCCOC(=O)C=C)ccc5c4)cc3SC)ccc2c1] | K91N215I |
| RM-31 | |
| 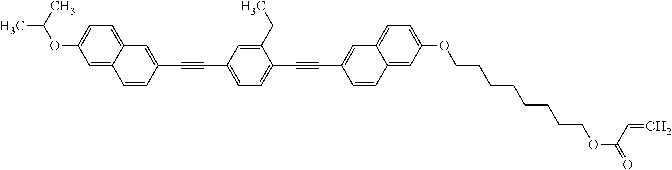 [SMILES: C=CC(=O)OCCCCCCCCOc1ccc2cc(C#Cc3ccc(C#Cc4ccc5cc(OC(C)C)ccc5c4)cc3CC)ccc2c1] | K106N208I |
| RM-32 | |
| 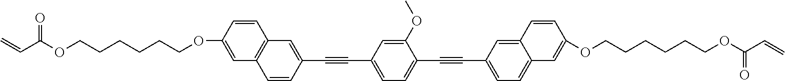 [SMILES: C=CC(=O)OCCCCCCOc1ccc2cc(C#Cc3ccc(C#Cc4ccc5cc(OCCCCCCOC(=O)C=C)ccc5c4)c(OC)c3)ccc2c1] | K19N198I |
| RM-33 | |
| 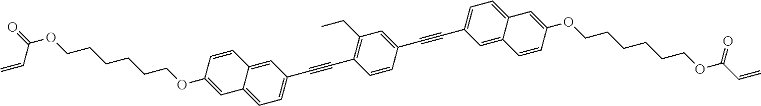 [SMILES: C=CC(=O)OCCCCCCOc1ccc2cc(C#Cc3ccc(C#Cc4ccc5cc(OCCCCCCOC(=O)C=C)ccc5c4)c(CC)c3)ccc2c1] | K80N196I |
| RM-34 | |
| 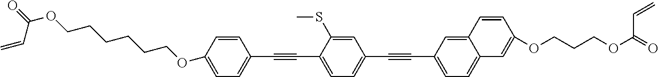 [SMILES: C=CC(=O)OCCCCCCOc1ccc(C#Cc2ccc(C#Cc3ccc4cc(OCCCOC(=O)C=C)ccc4c3)cc2SC)cc1] | |
| RM-35 | |
| 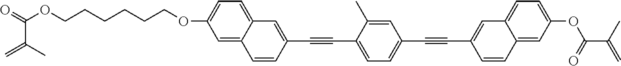 [SMILES: C=C(C)C(=O)OCCCCCCOc1ccc2cc(C#Cc3ccc(C#Cc4ccc5cc(OC(=O)C(=C)C)ccc5c4)cc3C)ccc2c1] | K172N284I |
| RM-36 | |
| 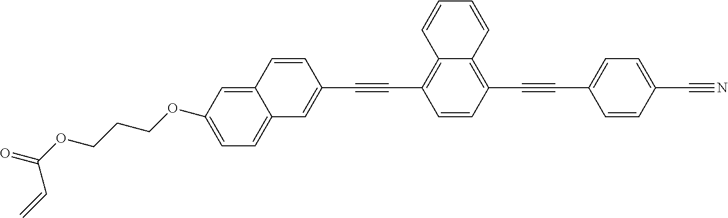 [SMILES: C=CC(=O)OCCCOc1ccc2cc(C#Cc3ccc(C#Cc4ccc(C#N)cc4)c4ccccc34)ccc2c1] | |
| RM-37 | |
| 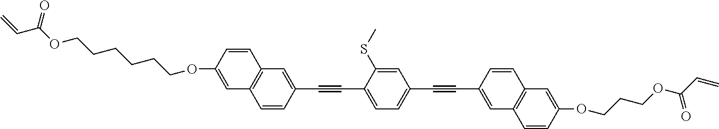 [SMILES: C=CC(=O)OCCCCCCOc1ccc2cc(C#Cc3ccc(C#Cc4ccc5cc(OCCCOC(=O)C=C)ccc5c4)cc3SC)ccc2c1] | |
| RM-38 | |
| 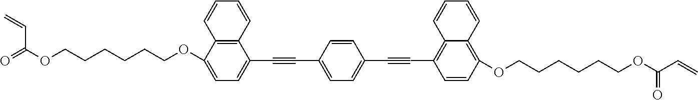 [SMILES: C=CC(=O)OCCCCCCOc1ccc(C#Cc2ccc(C#Cc3ccc(OCCCCCCOC(=O)C=C)c4ccccc34)cc2)c2ccccc12] | |
| RM-39 | |
| 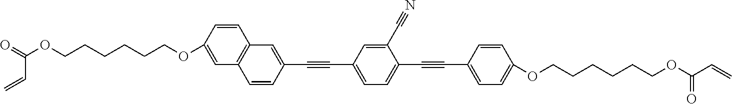 [SMILES: C=CC(=O)OCCCCCCOc1ccc(C#Cc2ccc(C#Cc3ccc4cc(OCCCCCCOC(=O)C=C)ccc4c3)cc2C#N)cc1] | K92SmX130N1 85I |
| RM-40 | |
| 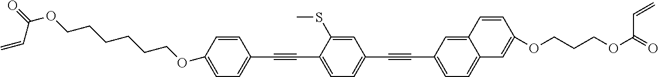 [SMILES: C=CC(=O)OCCCCCCOc1ccc(C#Cc2ccc(C#Cc3ccc4cc(OCCCOC(=O)C=C)ccc4c3)cc2SC)cc1] | |
| RM-41 | |
| 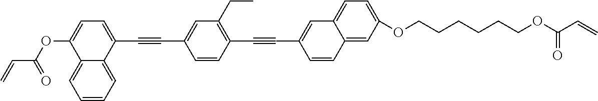 [SMILES: C=CC(=O)OCCCCCCOc1ccc2cc(C#Cc3ccc(C#Cc4ccc(OC(=O)C=C)c5ccccc45)cc3CC)ccc2c1] | K112N167I |
| RM-42 | |
| 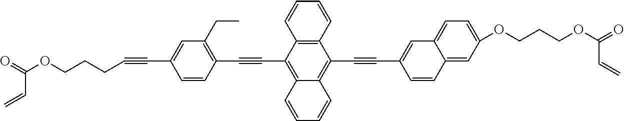 [SMILES: C=CC(=O)OCCCC#Cc1ccc(C#Cc2c3ccccc3c(C#Cc3ccc4cc(OCCCOC(=O)C=C)ccc4c3)c3ccccc23)c(CC)c1] | K105N120I |
| RM-43 | |
| 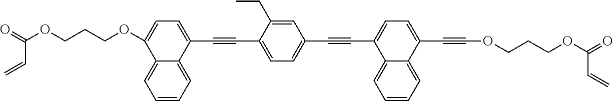 [SMILES: C=CC(=O)OCCCOC#Cc1ccc(C#Cc2ccc(C#Cc3ccc(OCCCOC(=O)C=C)c4ccccc34)c(CC)c2)c2ccccc12] | K94I |
| RM-44 | |
| 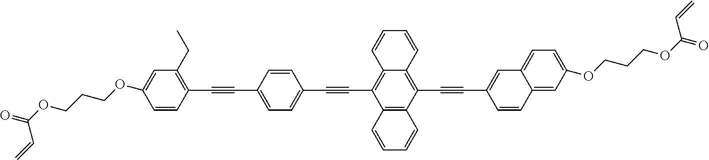 [SMILES: C=CC(=O)OCCCOc1ccc(C#Cc2ccc(C#Cc3c4ccccc4c(C#Cc4ccc5cc(OCCCOC(=O)C=C)ccc5c4)c4ccccc34)cc2)c(CC)c1] | |
| RM-45 | |
| 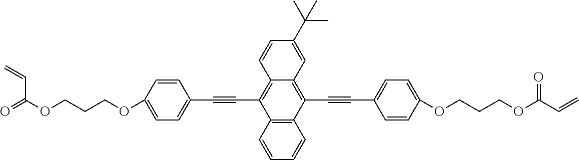 [SMILES: C=CC(=O)OCCCOc1ccc(C#Cc2c3ccccc3c(C#Cc3ccc(OCCCOC(=O)C=C)cc3)c3cc(C(C)(C)C)ccc23)cc1] | K155I |
| RM-46 | |
| 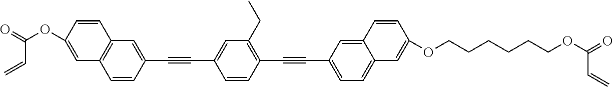 [SMILES: C=CC(=O)OCCCCCCOc1ccc2cc(C#Cc3ccc(C#Cc4ccc5cc(OC(=O)C=C)ccc5c4)cc3CC)ccc2c1] | |
| RM-47 | |
| 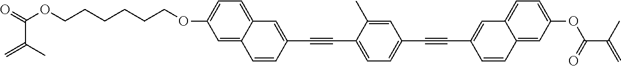 [SMILES: C=C(C)C(=O)OCCCCCCOc1ccc2cc(C#Cc3ccc(C#Cc4ccc5cc(OC(=O)C(=C)C)ccc5c4)cc3C)ccc2c1] | |
| RM-48 | |
| 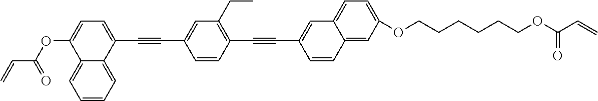 [SMILES: C=CC(=O)OCCCCCCOc1ccc2cc(C#Cc3ccc(C#Cc4ccc(OC(=O)C=C)c5ccccc45)cc3CC)ccc2c1] | |
| RM-49 | |
| 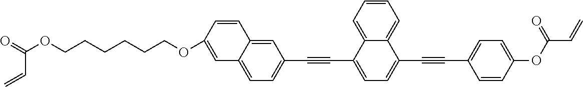 [SMILES: C=CC(=O)OCCCCCCOc1ccc2cc(C#Cc3ccc(C#Cc4ccc(OC(=O)C=C)cc4)c4ccccc34)ccc2c1] | |
| RM-50 | |
| 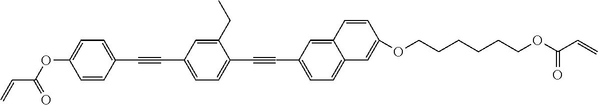 [SMILES: C=CC(=O)OCCCCCCOc1ccc2cc(C#Cc3ccc(C#Cc4ccc(OC(=O)C=C)cc4)cc3CC)ccc2c1] | |
| RM-51 | |
| 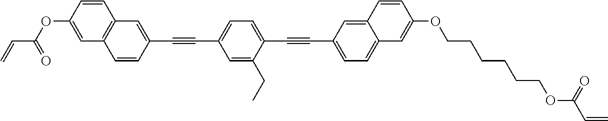 [SMILES: C=CC(=O)OCCCCCCOc1ccc2cc(C#Cc3ccc(C#Cc4ccc5cc(OC(=O)C=C)ccc5c4)cc3CC)ccc2c1] | |
| RM-52 | |
| 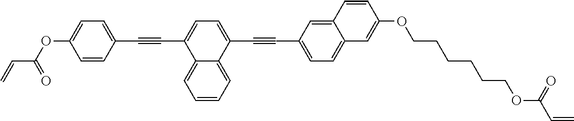 [SMILES: C=CC(=O)OCCCCCCOc1ccc2cc(C#Cc3ccc(C#Cc4ccc(OC(=O)C=C)cc4)c4ccccc34)ccc2c1] | |
| RM-53 | |
| 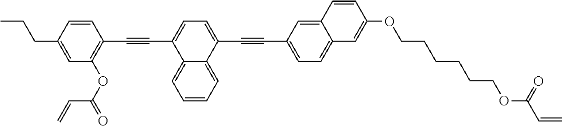 [SMILES: C=CC(=O)OCCCCCCOc1ccc2cc(C#Cc3ccc(C#Cc4ccc(CCC)cc4OC(=O)C=C)c4ccccc34)ccc2c1] | |
| RM-54 | |
| 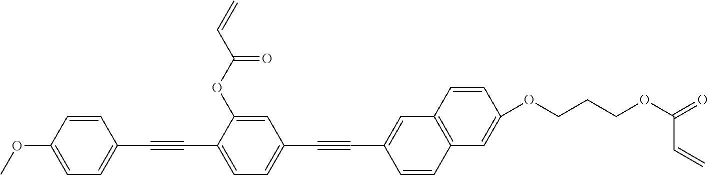 [SMILES: C=CC(=O)OCCCOc1ccc2cc(C#Cc3ccc(C#Cc4ccc(OC)cc4)c(OC(=O)C=C)c3)ccc2c1] | |
| RM-55 | |
| 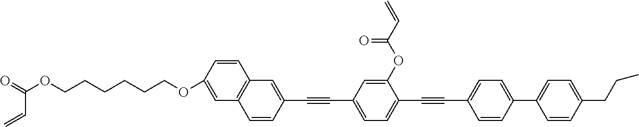 [SMILES: C=CC(=O)OCCCCCCOc1ccc2cc(C#Cc3ccc(C#Cc4ccc(-c5ccc(CCC)cc5)cc4)c(OC(=O)C=C)c3)ccc2c1] | K117N225I |
| RM-56 | |
| 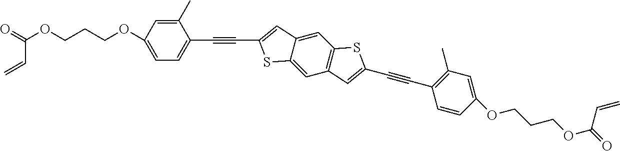 [SMILES: C=CC(=O)OCCCOc1ccc(C#Cc2cc3cc4sc(C#Cc5ccc(OCCCOC(=O)C=C)cc5C)cc4cc3s2)c(C)c1] | K174N229I |
| RM-57 | |
| 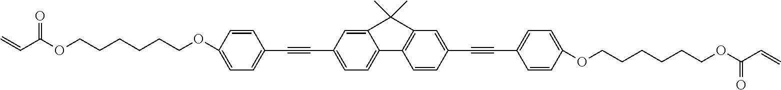 [SMILES: C=CC(=O)OCCCCCCOc1ccc(C#Cc2ccc3c(c2)C(C)(C)c2cc(C#Cc4ccc(OCCCCCCOC(=O)C=C)cc4)ccc2-3)cc1] | K106N155I |
| RM-58 | |
| 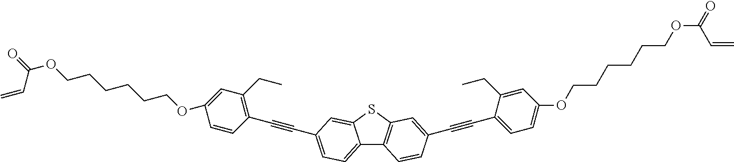 [SMILES: C=CC(=O)OCCCCCCOc1ccc(C#Cc2ccc3c(c2)sc2cc(C#Cc4ccc(OCCCCCCOC(=O)C=C)cc4CC)ccc23)c(CC)c1] | K130N160I |
| RM-59 | |
| 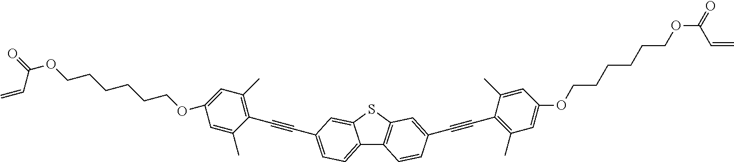 [SMILES: C=CC(=O)OCCCCCCOc1cc(C)c(C#Cc2ccc3c(c2)sc2cc(C#Cc4c(C)cc(OCCCCCCOC(=O)C=C)cc4C)ccc23)c(C)c1] | |
| RM-60 | |
| 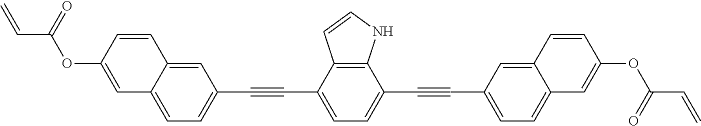 [SMILES: C=CC(=O)Oc1ccc2cc(C#Cc3ccc(C#Cc4ccc5cc(OC(=O)C=C)ccc5c4)c4[nH]ccc34)ccc2c1] | |
| RM-61 | |
| 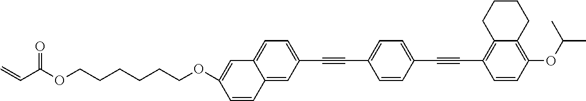 [SMILES: C=CC(=O)OCCCCCCOc1ccc2cc(C#Cc3ccc(C#Cc4ccc(OC(C)C)c5c4CCCC5)cc3)ccc2c1] | K115N184I |
| RM-62 | |
| 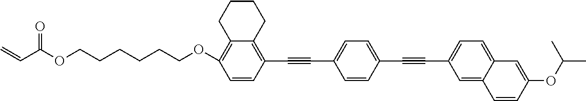 [SMILES: C=CC(=O)OCCCCCCOc1ccc(C#Cc2ccc(C#Cc3ccc4cc(OC(C)C)ccc4c3)cc2)c2c1CCCC2] | |
| RM-63 | |
| 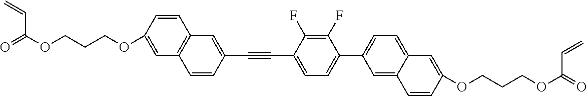 [SMILES: C=CC(=O)OCCCOc1ccc2cc(C#Cc3ccc(-c4ccc5cc(OCCCOC(=O)C=C)ccc5c4)c(F)c3F)ccc2c1] | K98SmX130N3 00I |
| RM-64 | |
| 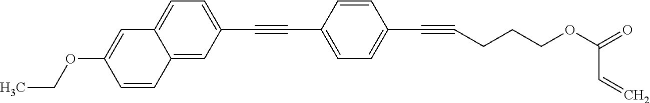 [SMILES: C=CC(=O)OCCCC#Cc1ccc(C#Cc2ccc3cc(OCC)ccc3c2)cc1] | K177I |
| RM-65 | |
| 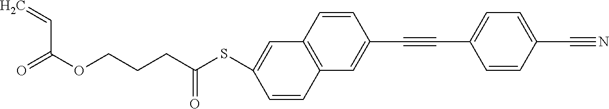 [SMILES: C=CC(=O)OCCCC(=O)Sc1ccc2cc(C#Cc3ccc(C#N)cc3)ccc2c1] | K94N118I |
| RM-66 | |
| 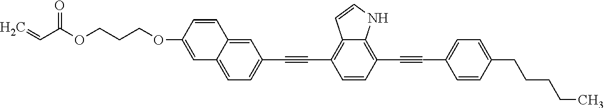 [SMILES: C=CC(=O)OCCCOc1ccc2cc(C#Cc3ccc(C#Cc4ccc(CCCCC)cc4)c4[nH]ccc34)ccc2c1] | K142N236I |
| RM-67 | |
| 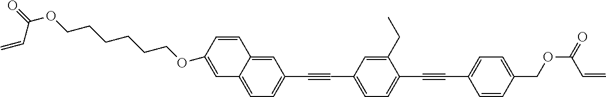 [SMILES: C=CC(=O)OCCCCCCOc1ccc2cc(C#Cc3ccc(C#Cc4ccc(COC(=O)C=C)cc4)c(CC)c3)ccc2c1] | |
| RM-68 | |
| 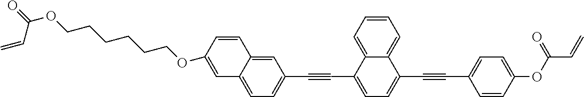 [SMILES: C=CC(=O)OCCCCCCOc1ccc2cc(C#Cc3ccc(C#Cc4ccc(OC(=O)C=C)cc4)c4ccccc34)ccc2c1] | K115N256I |
| RM-69 | |

### Mixture and Polymer Film Examples

Mixture M1 is prepared with the following composition:

### Mixture M1

| Material | Conc. |
|---|---|
| BYK361 | 0.20% |
| Irgacure 651 | 1.00% |
| Irganox 1076 | 0.08% |
| 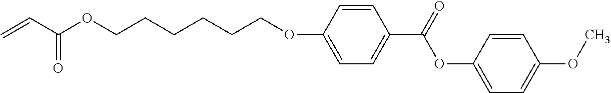 | 2.50% |
| 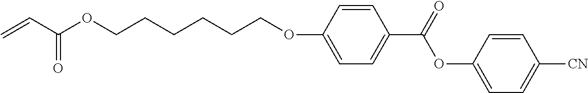 | 2.50% |
| 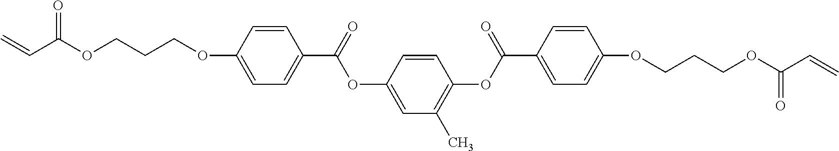 | 15.36% |
| 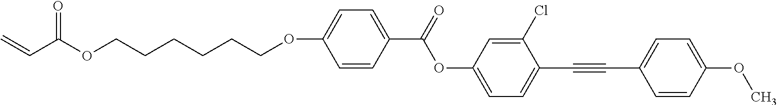 | 21.86% |
| 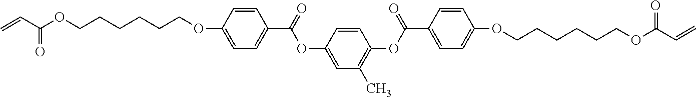 | 4.00% |
| 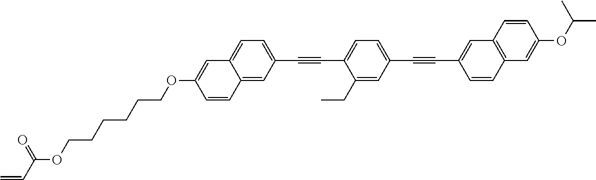 | 52.50% |
| (RM-1) | |

The additives BYK361, Irgacure® 651, and Irganox® 1076 are commercially available from Byk Gulden, Germany and CIBA, Switzerland.

### Preparation of Polymer Films

The mixture is dissolved in 1:2:1 MEK:cyclopentanone: MIBK to afford a 20% solids solution and the following process is applied:
Spin coat onto substrate for 30 seconds at 3000 rpm
Anneal for 60 sec at 66° C.
UV exposure, high pressure mercury lamp 250-450 nm (Dr. Hoenle), 80 mW/cm² for 60 seconds under a nitrogen atmosphere

The retardance of the resultant polymer film is measured by ellipsometry and the thickness then measured by profilometry. The resulting data are then used to calculate the birefringence. The average birefringence at 550 nm and 20° C. is thus determined to be 0.547.

Further mixtures M2 to M25 are prepared by replacing the compound RM-1 in Example Mixture M1 by the same amount of a compound selected from the compound examples listed above and as shown in Table 1.

Polymer films are prepared and the birefringence is determined as described above. The results are shown in Table 1.

TABLE 1

Polymer film

| | | Birefringence |
|---|---|---|
| Mixture | Compound | at 550 nm |
| M1 | RM-1 | 0.547 |
| M2 | RM-2 | 0.489 |
| M3 | RM-4 | 0.450 |
| M4 | RM-6 | 0.354 |
| M5 | RM-7 | 0.376 |
| M6 | RM-8 | 0.432 |
| M7 | RM-10 | 0.353 |
| M8 | RM-12 | 0.336 |
| M9 | RM-13 | 0.547 |
| M10 | RM-20 | 0.378 |
| M11 | RM-23 | 0.336 |
| M12 | RM-24 | 0.444 |
| M13 | RM-26 | 0.351 |
| M14 | RM-27 | 0.432 |
| M15 | RM-29 | 0.339 |
| M16 | RM-30 | 0.406 |
| M27 | RM-32 | 0.426 |
| M18 | RM-33 | 0.364 |
| M19 | RM-34 | 0.370 |
| M20 | RM-35 | 0.336 |
| M21 | RM-40 | 0.346 |
| M22 | RM-56 | 0.393 |
| M23 | RM-64 | 0.335 |
| M24 | RM-67 | 0.435 |
| M25 | RM-69 | 0.392 |

### Example Mixture M26

| Material | Conc. |
|---|---|
| POLYFOX™ PF-656 | 0.20% |
| Irganox 1076 | 0.08% |
| TR-PBG-304 | 3.25% |
| NCI-803 | 3.25% |
| 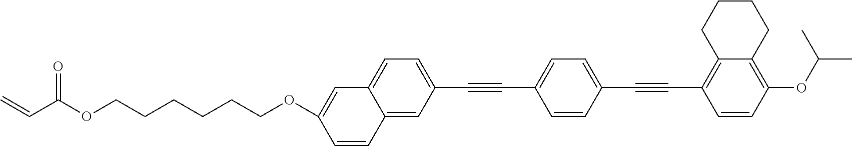 [SMILES: C=CC(=O)OCCCCCCOc1ccc2cc(C#Cc3ccc(C#Cc4ccc(OC(C)C)c5c4CCCC5)cc3)ccc2c1] | 4.30% |
| (RM-62) | |
| 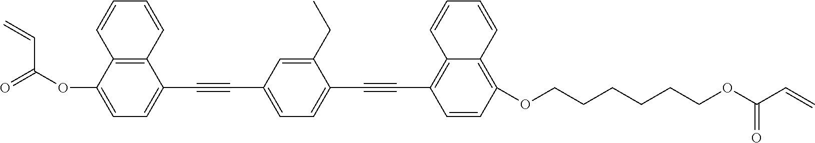 [SMILES: C=CC(=O)OCCCCCCOc1ccc(C#Cc2ccc(C#Cc3ccc(OC(=O)C=C)c4ccccc34)cc2CC)c2ccccc12] | 10.60% |
| (RM-15) | |
| 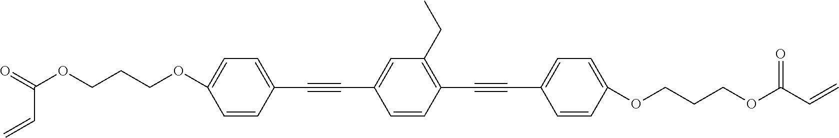 [SMILES: C=CC(=O)OCCCOc1ccc(C#Cc2ccc(C#Cc3ccc(OCCCOC(=O)C=C)cc3)c(CC)c2)cc1] | 13.70% |
| (RM-17) | |
| 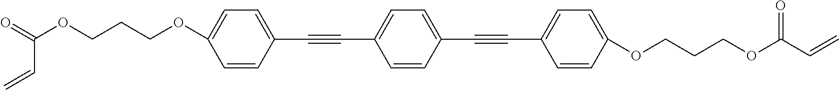 [SMILES: C=CC(=O)OCCCOc1ccc(C#Cc2ccc(C#Cc3ccc(OCCCOC(=O)C=C)cc3)cc2)cc1] | 25.00% |
| 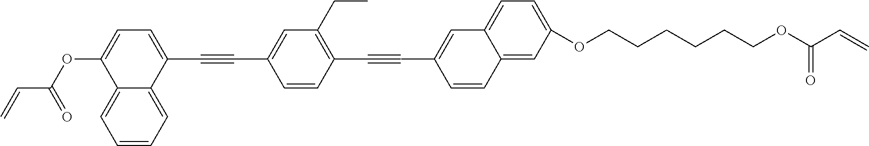 [SMILES: C=CC(=O)OCCCCCCOc1ccc2cc(C#Cc3ccc(C#Cc4ccc(OC(=O)C=C)c5ccccc45)cc3CC)ccc2c1] | 39.62% |
| (RM-42) | |

POLYFOX™ PF-656 is commercially available from Synthomer plc, TR-PBG-304 is commercially available from Changzou Tronly, and NCI-803 is commercially available from Adeka.

The mixture is dissolved in 1:2:1:2 MIBK:Cyclopentanone:MEK:3-Undecan-one to afford a 20% solids solution and a polymer film is prepared as follows:
Spin coat onto substrate for 30 seconds at 3000 rpm
Anneal for 60 sec at 90° C.
UV exposure, high pressure mercury lamp 250-450 nm (Dr. Hoenle), 80 mW/cm² for 60 seconds under a nitrogen athmosphere

The birefringence of the polymer film is determined as described above at 550 nm and 20° C. to be 0.375.

### Example Mixture M27

| Material | Conc. |
|---|---|
| PF-656 | 0.20% |
| Irganox 1076 | 0.10% |
| TR-PBG-304 | 2.50% |
| NCI-803 | 2.50% |
| 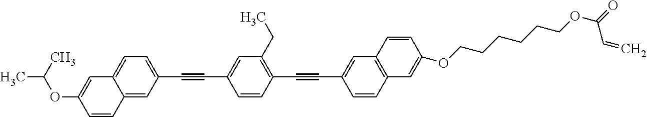 [SMILES: C=CC(=O)OCCCCCCOc1ccc2cc(C#Cc3ccc(C#Cc4ccc5cc(OC(C)C)ccc5c4)cc3CC)ccc2c1] | 16.70% |
| (RM-13) | |
| 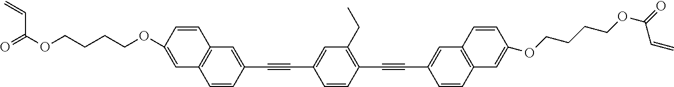 [SMILES: C=CC(=O)OCCCCOc1ccc2cc(C#Cc3ccc(C#Cc4ccc5cc(OCCCCOC(=O)C=C)ccc5c4)c(CC)c3)ccc2c1] | 40.00% |
| (RM-12) | |
| 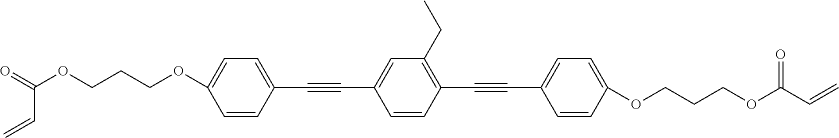 [SMILES: C=CC(=O)OCCCOc1ccc(C#Cc2ccc(C#Cc3ccc(OCCCOC(=O)C=C)cc3)c(CC)c2)cc1] | 22.00% |
| (RM-17) | |
| 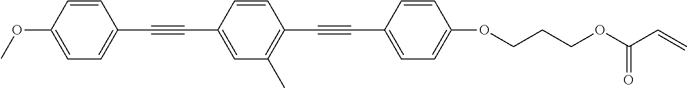 [SMILES: C=CC(=O)OCCCOc1ccc(C#Cc2ccc(C#Cc3ccc(OC)cc3)cc2C)cc1] | 16.00% |

### Example Mixture M28

| Material | Conc. |
|---|---|
| PF-656 | 0.20% |
| Irganox 1076 | 0.10% |
| TR-PBG-304 | 2.50% |
| NCI-803 | 2.50% |
| 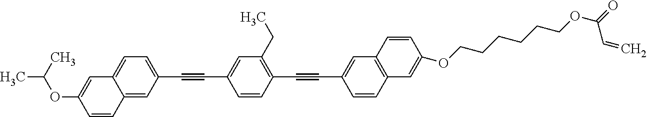 [SMILES: C=CC(=O)OCCCCCCOc1ccc2cc(C#Cc3ccc(C#Cc4ccc5cc(OC(C)C)ccc5c4)cc3CC)ccc2c1] | 16.70% |
| (RM-13) | |
| 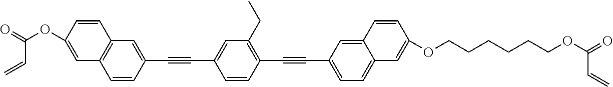 [SMILES: C=CC(=O)OCCCCCCOc1ccc2cc(C#Cc3ccc(C#Cc4ccc5cc(OC(=O)C=C)ccc5c4)cc3CC)ccc2c1] | 40.00% |
| (RM-47) | |
| 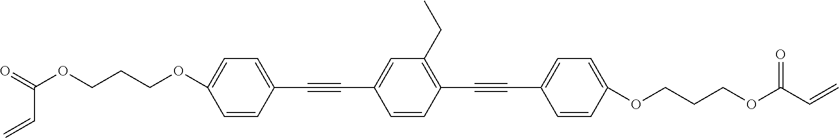 [SMILES: C=CC(=O)OCCCOc1ccc(C#Cc2ccc(C#Cc3ccc(OCCCOC(=O)C=C)cc3)c(CC)c2)cc1] | 22.00% |
| (RM-17) | |
| 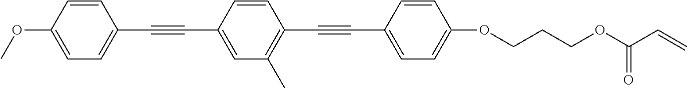 [SMILES: C=CC(=O)OCCCOc1ccc(C#Cc2ccc(C#Cc3ccc(OC)cc3)cc2C)cc1] | 16.00% |

Preparation of Polymer films 1

The mixtures M27 and M28 are each dissolved to give a 20% solids mixture in 1:2:1 MIBK: Cyclopentanone: MEK. A film of each mixture is then prepared via spin coating on a KBr disc and annealing the film at 80° C. for 30 s. The uncured films are then placed in FTIR analysis chamber purged with N2. IR spectra of the films are recorded at 2 second intervals over a period of 2 minutes. After 30 seconds of measurement the films are irradiated with broadband UV light (250-450 nm, 60 mWcm⁻²@ 365 nm) for 1 minute to form cured polymer films.

The resulting spectra are analysed by measuring the area of the carbonyl peak (1800-1735 cm⁻¹λₘₐₓ=1735 cm⁻¹) and acrylic C═C bond (1646-1613 cm⁻¹ λₘₐₓ=1629 cm⁻¹). To compare the relative peak intensity over time an intensity value is calculated as the product of two peak areas on the spectrum (ρ). This value is then converted to degree of cure via equation 1. The resulting data is shown in Table 1.

$$Cure \% = 1 - \frac{{ρ}_{t}}{{ρ}_{i}}$$

Equation 1: Formular calculating degree of cure in %, ρₜ—intensity product at time t, ρᵢ—initial intensity product

Due to overlap in the overlap of aromatic C═C stretches with the acrylic C═C, in this case, it is not possible for this peak to be completely removed and the measured value does not reach 100% cure despite well cured hard films being obtained. For comparison the calculated values are then normalised, assigning the highest level of cure obtained as 100%.

TABLE 1

UV Time / s

| | | | | | |
|---|---|---|---|---|---|
| 20 | 40 | 60 | 80 | 100 | 120 |

Norm. Cure / %

| | | | | | | |
|---|---|---|---|---|---|---|
| Mixture 15 | 0 | 64 | 76 | 84 | 86 | 83 |
| Mixture 16 | 0 | 73 | 91 | 95 | 97 | 97 |

It can be seen from Table 1 that Mixture 28 with compound RM-47, which has only one spacer group, reaches a normalised degree of cure which is 10 to 15% higher compared to Mixture 27 with the same composition except that compound RM-47 is replaced by the corresponding amount of compound RM-12 having homologous structure but with a spacer group on each side of the mesogenic core. This indicates that compound RM-47 with only one spacer group does positively contribute to achieving a higher degree of cure.

Preparation of Polymer films 2

The mixtures M27 and M28 are each dissolved to give a 20% solids mixture in 1:2:1 MIBK: Cyclopentanone: MEK. A film of each of mixture is then spin coated on rubbed polyimide coated glass, the resulting films are polymerised by exposure to UV light (400 nm, 80 mWcm⁻², 60 s, under N₂). The retardance of the resultant polymer films is measured by ellipsometry and the thickness then measured by profilometry. The resulting data are then used to calculate the birefringence data shown in Table 2.

TABLE 2

| Wavelength / nm | Mixture M27 | Mixture M28 |
|---|---|---|
| 450 | 0.602 | 0.602 |
| 550 | 0.457 | 0.457 |
| 650 | 0.407 | 0.408 |

It can be seen from Table 2 that the polymer films prepared from mixtures M27 and M28 give almost identical optical performance, this is attributed to the common host mixture and homologous structures of RM-12 and RM-47.

The above results demonstrate that compounds of formula I having only one spacer group are especially suitable for preparing polymer films with both high birefringence and a high degree of cure.

1. A compound of formula I, 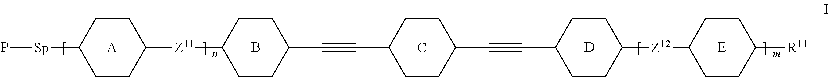
wherein
P is a polymerisable group,
Sp is a spacer group or a single bond,
R¹¹ is F, Cl, CN, NCS, alkyl, alkoxy, thioalkyl, alkylcarbonyl, alkoxycarbonyl, alkylcarbonyloxy or alkoxycarbonyloxy or P-Sp,
A, B,
D, and E denote, in case of multiple occurrence independently of one another, an alicyclic, heterocyclic, aromatic or heteroaromatic group with 4 to 20 ring atoms, which is monocyclic or polycyclic and which is optionally substituted by one or more groups L or P-Sp-, and one of rings C and D may also denote a single bond,
L F, Cl, —CN, P-Sp-, or straight chain alkyl having 1 to 25 C atoms or, branched or cyclic alkyl having 3 to 25 C atoms, wherein one or more non-adjacent CH₂-groups are optionally replaced by —O—, —S—, —CO—, —CO—O—, —O—CO—, —O—CO—O—, CR⁰═CR⁰⁰—, —C═C—, 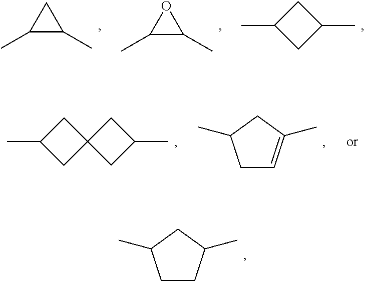
in such a manner that O- and/or S-atoms are not directly connected with each other, and wherein one or more H atoms are each optionally replaced by P-Sp-, F or Cl, or two substituents L that are connected to directly adjacent C atoms may also form a cycloalkyl or cycloalkenyl group with 5, 6, 7 or 8 C atoms,
C denotes 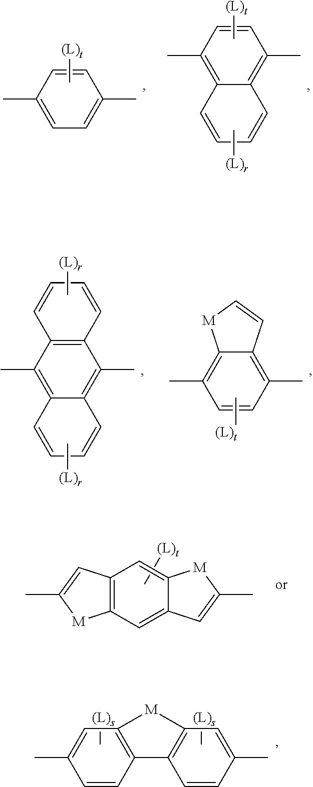
M denotes CH₂, C(CH₃)₂, CHF, CF₂, NH, S or O,
Z¹¹, Z¹² denotes, in case of multiple occurrence independently of one another, —O—, —S—, —CO—, —COO—, —OCO—, —S—CO—, —CO—S—, —O—COO—, —CO—NR⁰—, —NR⁰—CO—, —NR⁰—CO—NR⁰⁰, —NR⁰—CO—O—, —O—CO—NR⁰—, —OCH₂—, —CH₂O—, —SCH₂—, —CH₂S—, —CF₂O—, —OCF₂—, —CF₂S—, —SCF₂—, —CH₂CH₂—, —(CH₂)ₙ₁, —CF₂CH₂—, —CH₂CF₂—, —CF₂CF₂—, —CH═N—, —N═CH—, —N═N—, —CH═CR⁰—, —CY₁═CY₂—, —C═C—, —CH═CH—COO—, —OCO—CH═CH— or a single bond,
n1 is 1, 2, 3 or 4,
r denotes 0, 1, 2, 3 or 4,
s denotes 0, 1, 2 or 3,
t denotes 0, 1 or 2,
R⁰, R⁰⁰ denote H or alkyl having 1 to 12 C atoms,
Y¹, Y² independently of each other denote H, F, Cl, NCS, or CN,
n is 0, 1, 2, 3 or 4, and
m is 0, 1, 2, 3 or 4.

2. The compound according to claim 1, wherein one, two, three, four or more of rings A, B, D and/or E in formula I are selected from the group consisting of 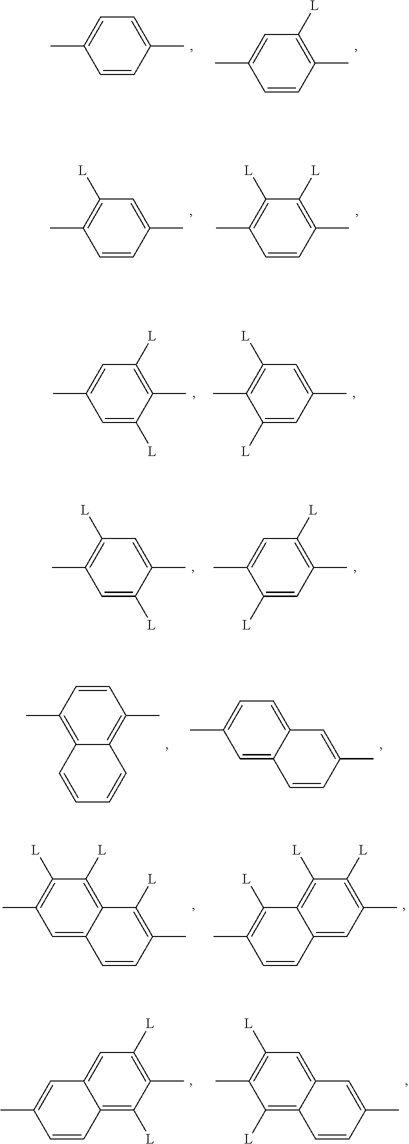 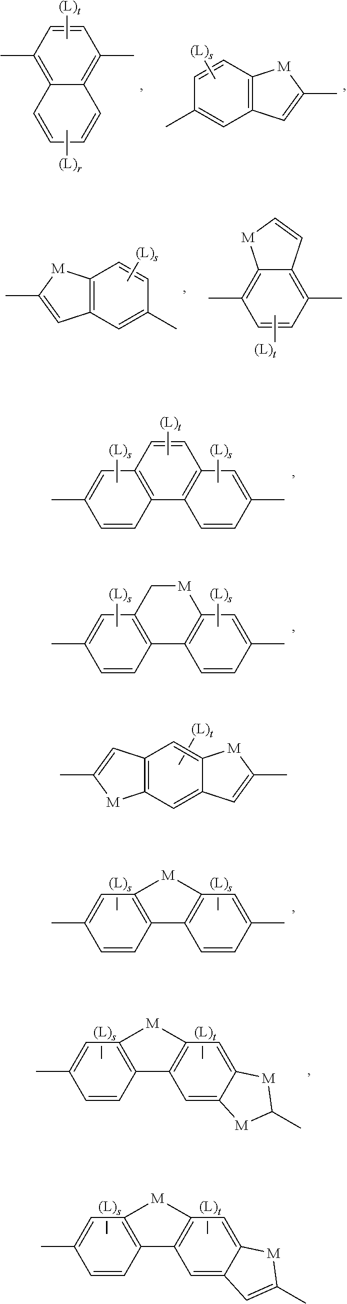
wherein
L each, independently of one another, denotes alkyl, alkoxy or thioalkyl having 1 to 6 C atoms, P-Sp-, —CN, F, Cl, OCF₃, CF₃, CH₂F, CHF₂, or two substituents L that are connected to directly adjacent C atoms may also form a cyclic group with 5, 6, 7 or 8 C atoms,
r denotes 0, 1, 2 or 3,
s denotes 0, 1 or 2,
t denotes 0, 1 or 2, and
M denotes CH₂, C(CH₃)₂, CHF, CF₂, NH, S or O.

3. The compound according to claim 1, wherein one or both of rings B and D in formula I are selected from the group consisting of 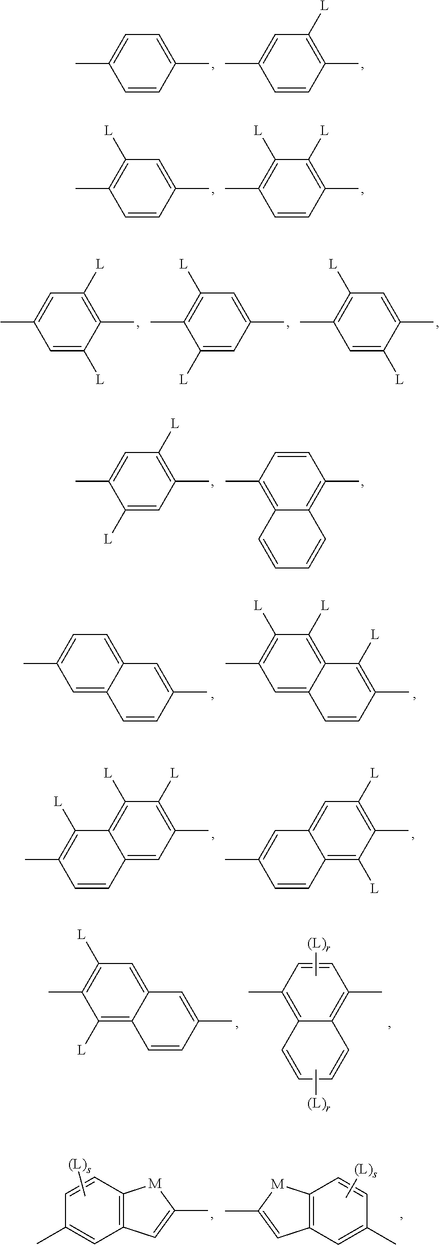 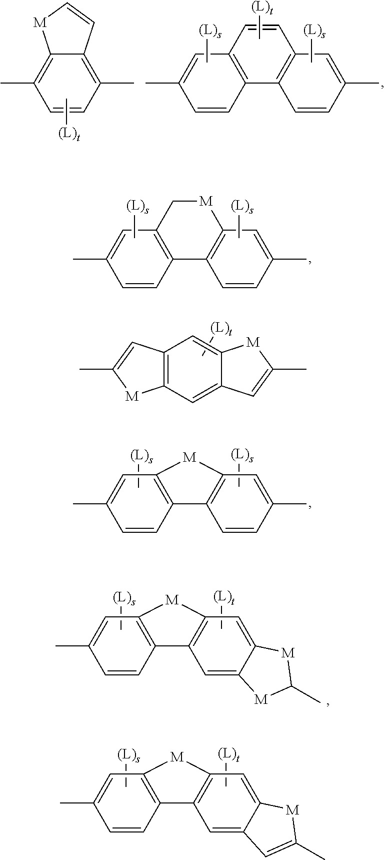
wherein
L each, independently of one another, denotes alkyl, alkoxy or thioalkyl having 1 to 6 C atoms, P-Sp-, —CN, F, Cl, OCF₃, CF₃, CH₂F, CHF₂, or two substituents L that are connected to directly adjacent C atoms may also form a cyclic group with 5, 6, 7 or 8 C atoms,
r denotes 0, 1, 2, or 3,
s denotes 0, 1 or 2,
t denotes 0, 1 or 2, and
M denotes CH₂, C(CH₃)₂, CHF, CF₂, NH, S or O.

4. The compound according to claim 1, wherein ring C in formula I is selected from the group consisting of 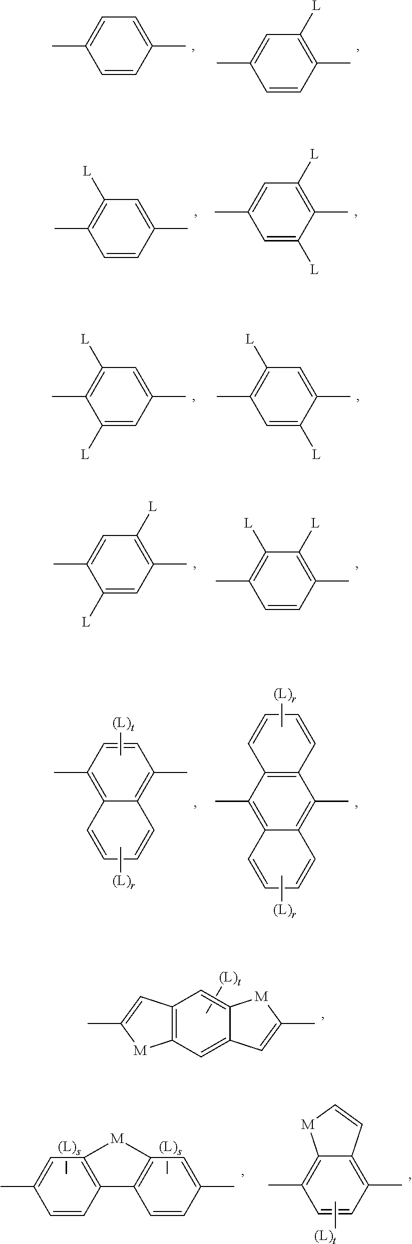
wherein
L each, independently of one another, denotes P-Sp-, F, —CN, alkyl, alkoxy or thioalkyl having 1 to 6 C atoms,
r denotes 0, 1, 2 or 3,
s denotes 0, 1, 2 or 3,
t denotes 0, 1 or 2,
M denotes CH₂, C(CH₃)₂, CHF, CF₂, NH, S or O, and
M¹ denotes S, O or NH.

5. The compound according to claim 1, wherein it is selected from the following subformulae: 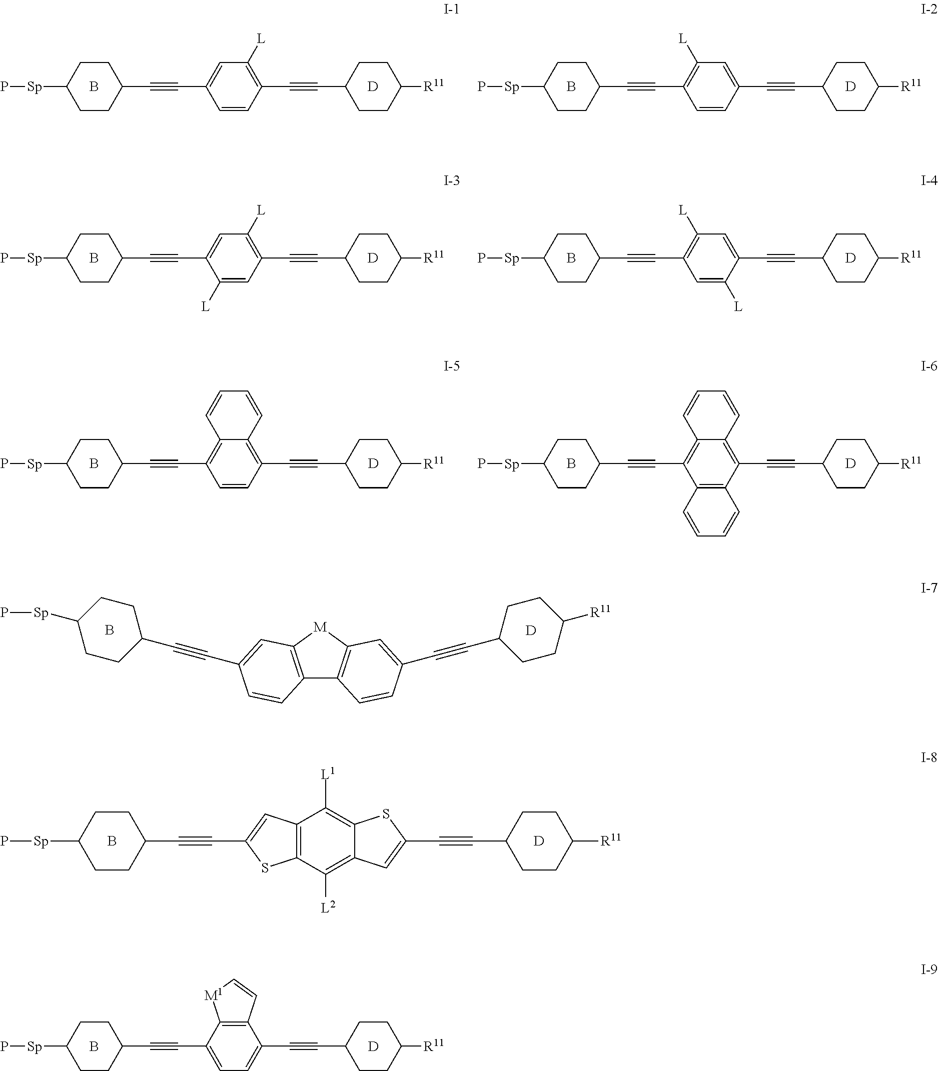
wherein
R¹¹ is F, Cl, CN, NCS, alkyl, alkoxy, thioalkyl, alkylcarbonyl, alkoxycarbonyl, alkylcarbonyloxy or alkoxycarbonyloxy or P-Sp,
B denotes, in case of multiple occurrence independently of one another, an alicyclic, heterocyclic, aromatic or heteroaromatic group with 4 to 20 ring atoms, which is monocyclic or polycyclic and which is optionally substituted by one or more groups L or P-Sp-,
C denotes 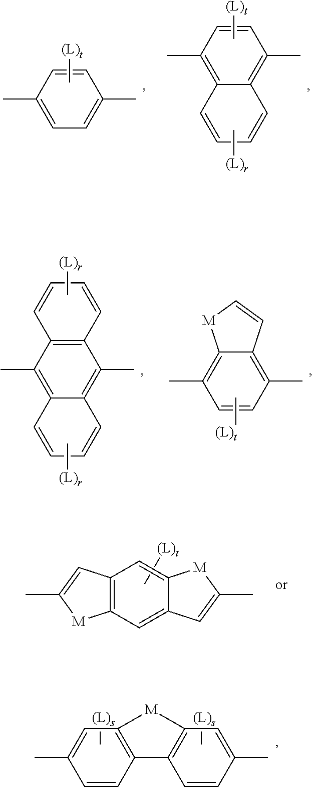
L each, independently of one another, denotes P-Sp-, F, CN, alkyl, alkoxy or thioalkyl having 1 to 6 C atoms,
L¹ and L² each, independently of one another, denote H or L,
M is S, O, NH, CH₂ or, C(CH₃)₂, and
M¹ is NH or S.

6. The compound according to claim 1, wherein it is selected from the following subformula: 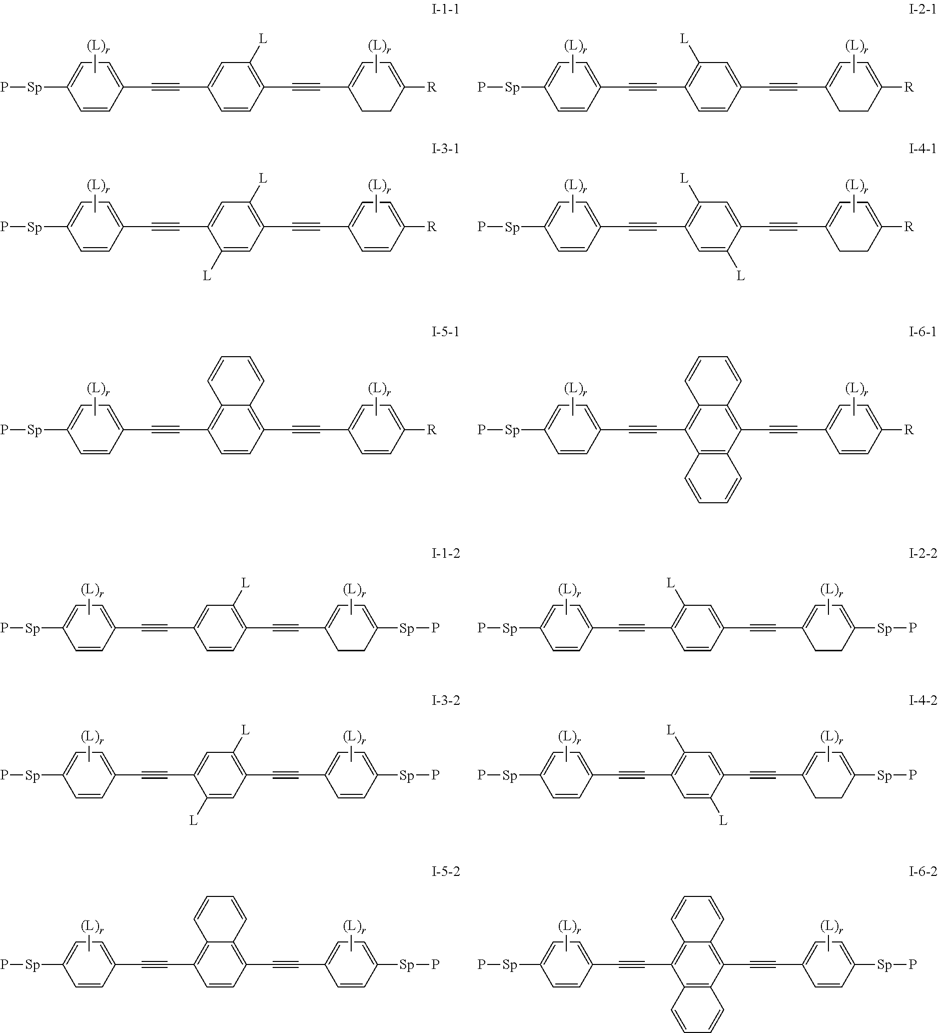 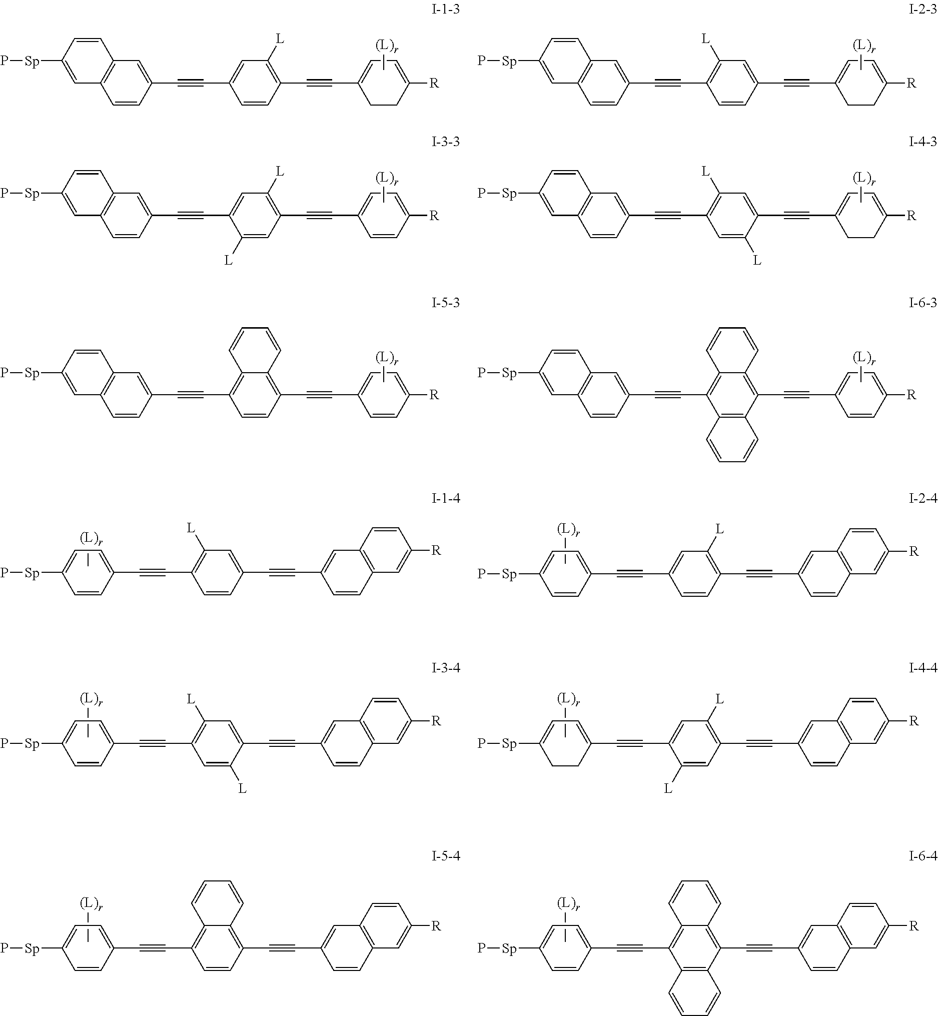 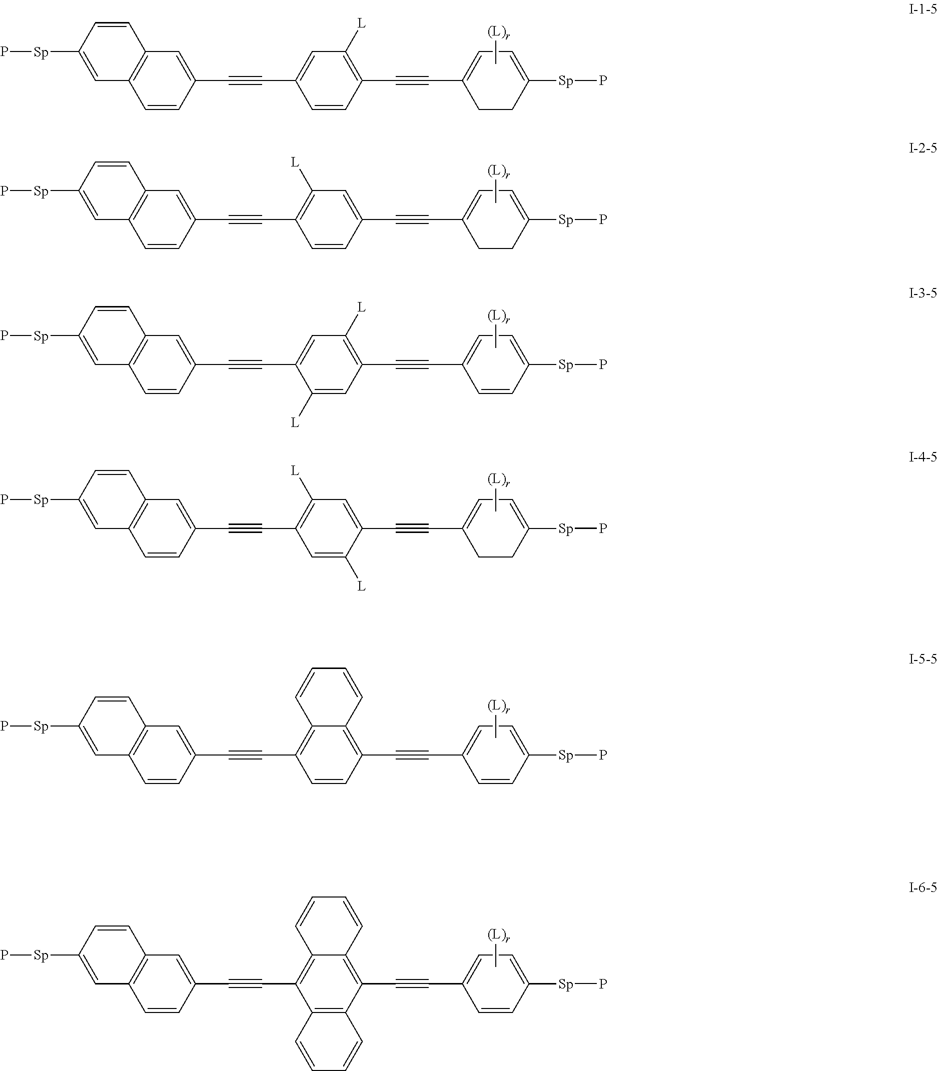 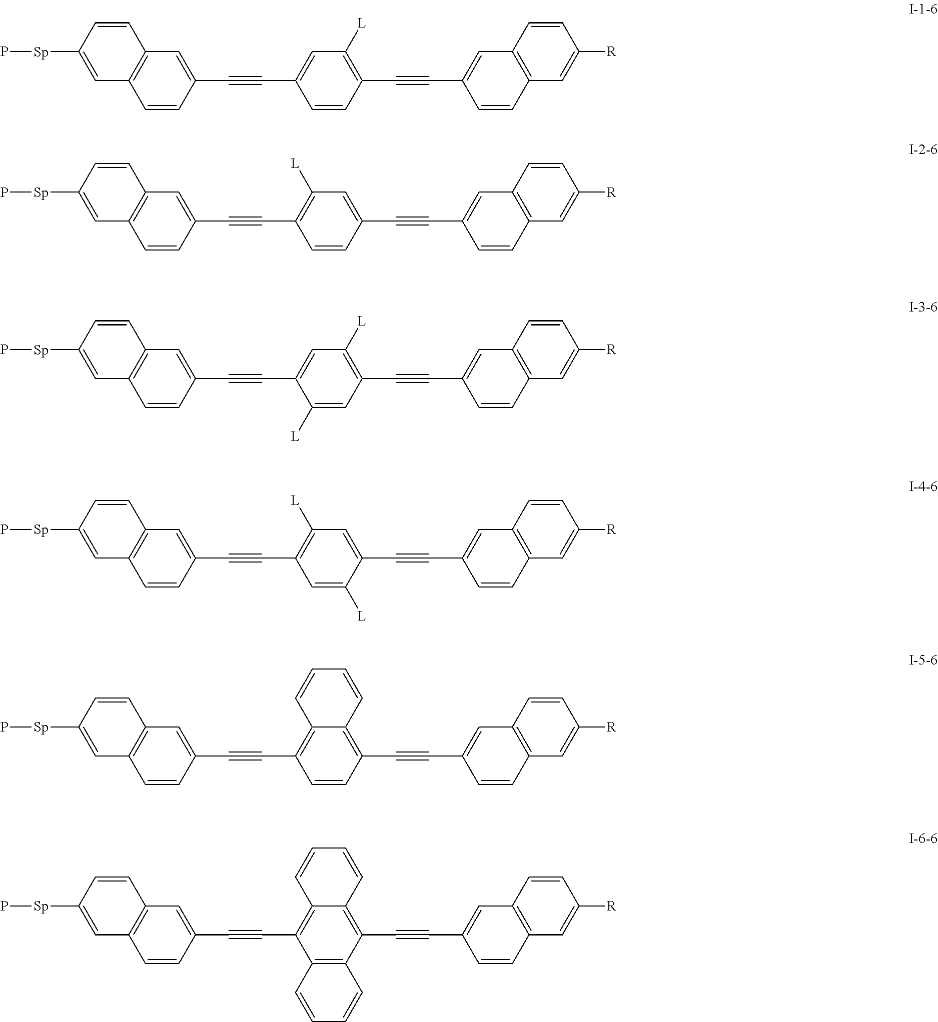 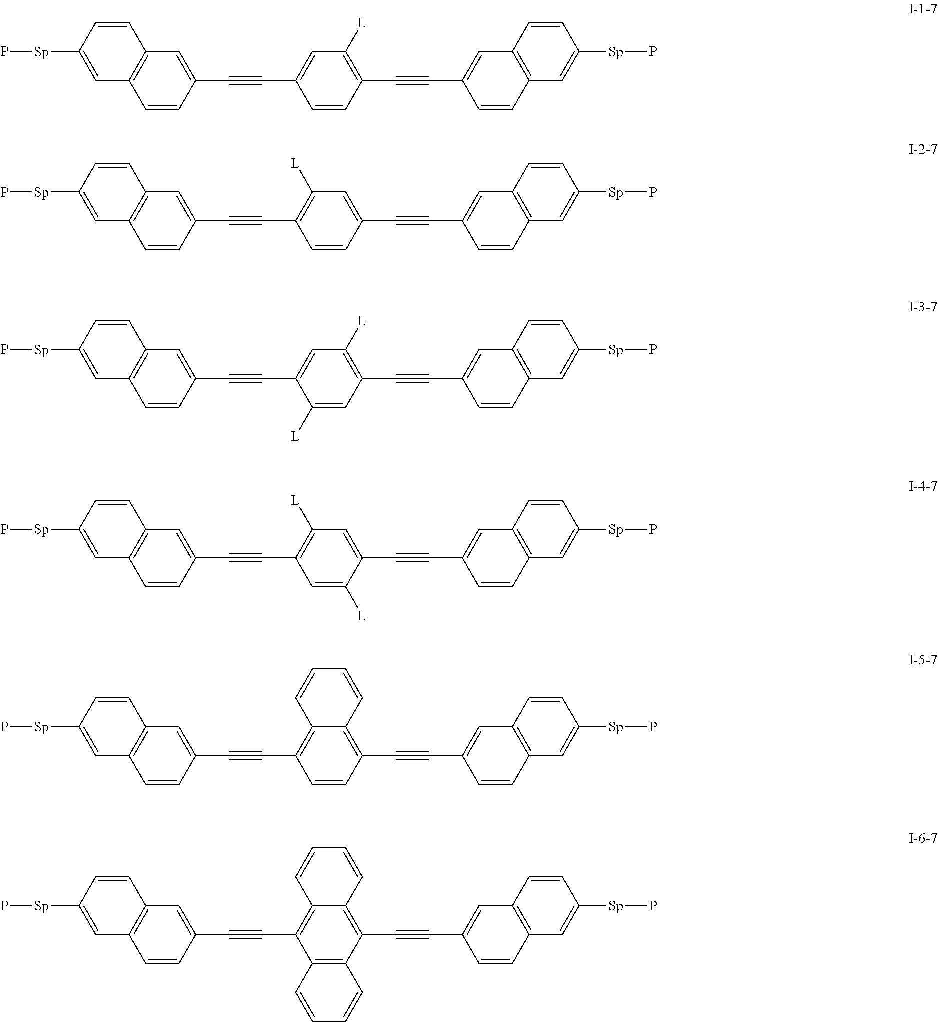 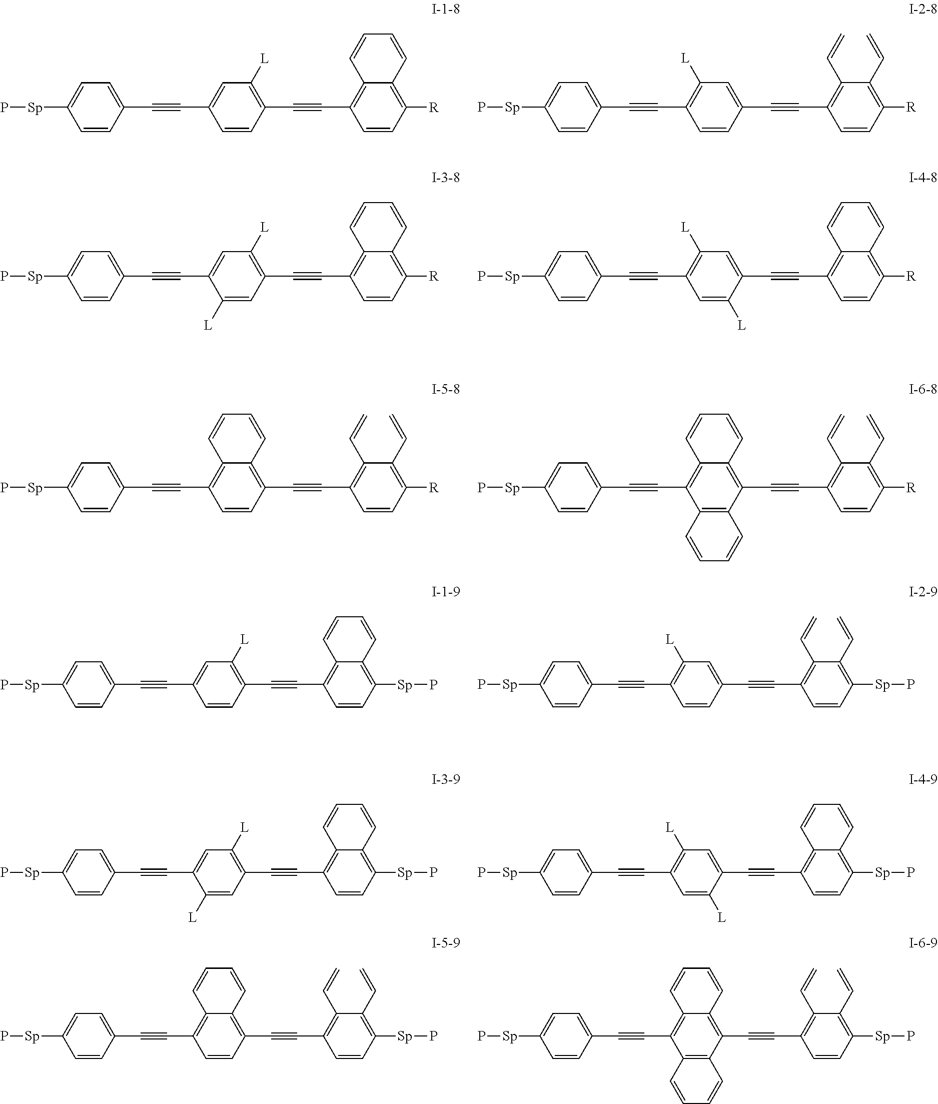 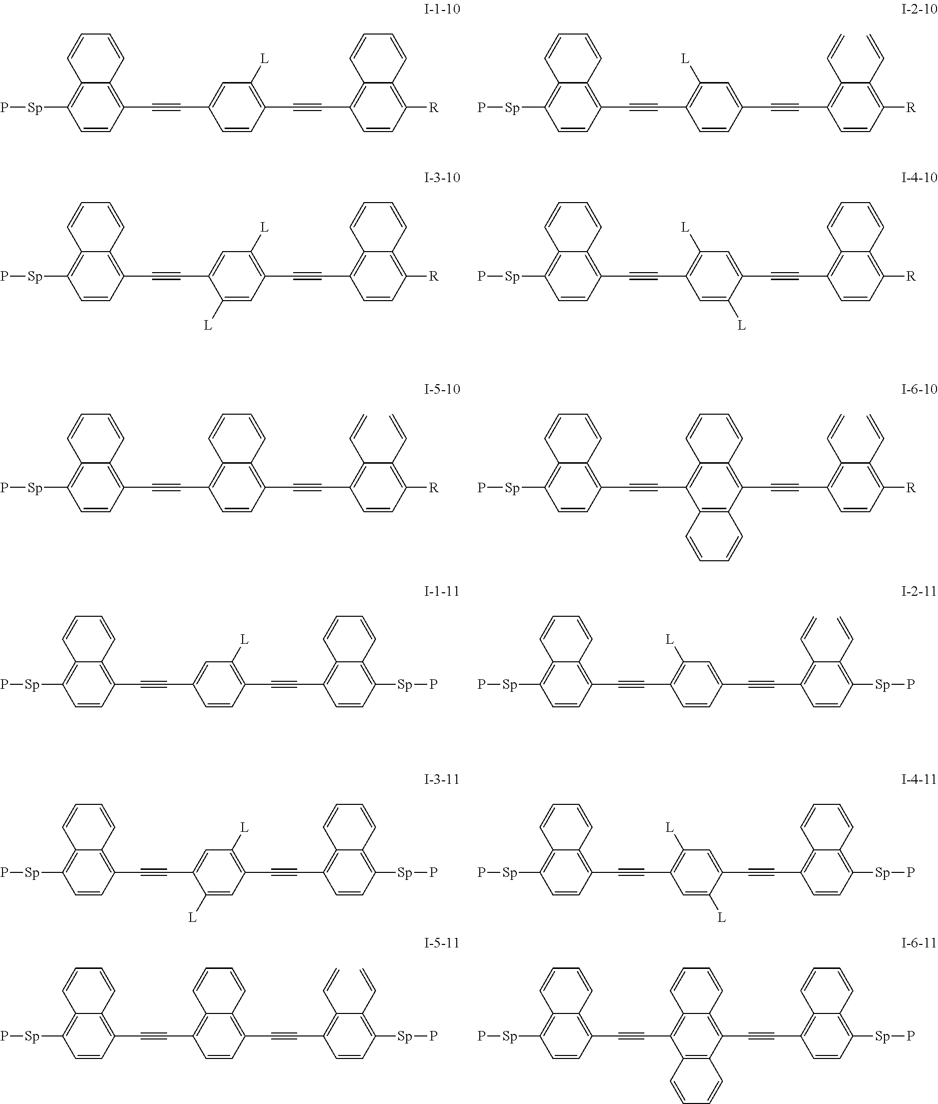
wherein
P is a polymerisable group,
Sp is a spacer group or a single bond,
R is alkyl, alkoxy, thioalkyl, alkylcarbonyl, alkoxycarbonyl, alkylcarbonyloxy or alkoxycarbonyloxy,
L P-Sp- is acrylate, F, Cl, —CN, or straight chain alkyl having 1 to 25 C atoms or branched or cyclic alkyl having 3 to 25 C atoms, wherein one or more non-adjacent CH₂-groups are optionally replaced by —O—, —S—, —CO—, —CO—O—, —O—CO—, —O—CO—O—, CR⁰═CR⁰⁰—, —C═C—, 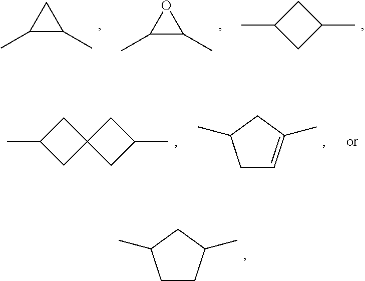
in such a manner that O- and/or S-atoms are not directly connected with each other, and wherein one or more H atoms are each optionally replaced by P-Sp-, F or Cl, and two substituents L that are connected to directly adjacent C atoms may also form a cyclic group with 5, 6, 7 or 8 C atoms, and
r denotes 0, 1, 2, 3 or 4, preferably 0, 1 or 2.

7. The compound according to claim 1, wherein P denotes an acrylate or methacrylate group

8. A mixture comprising two or more reactive mesogens (RMs), at least one of which is a compound of formula I according to claim 1.

9. The mixture according to claim 8, wherein it comprises one or more RMs having only one polymerisable functional group, and one or more RMs having two or more polymerisable functional groups.

10. A formulation comprising one or more compounds of formula I according to claim 1 and further comprising one or more solvents and/or additives.

11. A polymer obtainable by polymerising a compound of formula I according to claim 1.

12. An optical, electrooptical or electronic device or a component thereof, comprising a compound of formula I according to claim 1.

13. An optical, electrooptical or electronic device or a component thereof, comprising an RM mixture according to claim 8.

14. The component of claim 13, which is selected from optical retardation films, polarizers, compensators, beam splitters, reflective films, alignment layers, colour filters, antistatic protection sheets, electromagnetic interference protection sheets, polarization-controlled lenses, IR reflection films, and lenses for light guides, focusing and optical effects.

15. The device of claim 13, which is selected from LC displays, autostereoscopic 3D displays, organic light emitting diodes (OLEDs), optical data storage devices, googles for AR/VR applications and windows.

16. An optical, electrooptical or electronic device or a component thereof, comprising a polymer according to claim 11.
